(19) United States
(12) Patent Application Publication
(10) Pub. No.: US 2009/0130123 A1
(43) Pub. Date: May 21, 2009

(54) Antibodies to west nile virus polypeptides

(76) Inventors: Erol Fikrig, Guilford, CT (US); Hannah Gould, New Haven, CT (US); Raymond A. Koski, Old Lyme, CT (US); Michel Ledizet, Woodbridge, CT (US); Wayne A. Marasco, Wellesley, MA (US); Jianhua Sui, Boston, MA (US)

Correspondence Address: ROPES & GRAY LLP, PATENT DOCKETING 39/361, 1211 AVENUE OF THE AMERICAS, NEW YORK, NY, 10036-8704, US

(21) Appl. No.: 11/629,825

(22) PCT Filed: Jun. 15, 2005
(86) PCT No.: PCT/US05/22188

§ 371 Date: Sep. 19, 2008

Related U.S. Application Data
(60) Provisional application No. 60/580,248, filed on Jun. 15, 2004.
(60) Provisional application No. 60/613,369, filed on Sep. 27, 2004.
(60) Provisional application No. 60/646,839, filed on Jan. 24, 2005.

(51) Int. Cl. A61K 39/395; C07K 16/18; C12N 5/06; C12N 15/11; C12N 15/00; C12P 21/04; A01K 67/027; A01H 5/00; A61K 31/7088; C12Q 1/70

(52) U.S. Cl. 424/159.1; 530/387.1; 530/387.3; 435/339; 536/23.53; 435/320.1; 435/69.6; 800/298; 800/13; 514/44; 800/3; 435/5

(57) ABSTRACT

The present invention relates to anti-West Nile virus E protein (WNE) antibodies, including human antibodies, and antigen-binding portions thereof. In particular, the invention relates to such antibodies and portions that prevent, inhibit, or treat a flavivirus infection, including a West Nile Virus infection. The invention also relates to antibodies that are chimeric, bispecific, derivatized, single chain antibodies or that are portions of fusion proteins. The invention also relates to isolated heavy and light chain immunoglobulins derived from human anti-WNE antibodies and nucleic acid molecules encoding such immunoglobulins. The present invention also relates to methods of making human anti-WNE antibodies, compositions comprising these antibodies and methods of using the antibodies and compositions for diagnosis, prophylaxis and treatment. The invention also provides gene therapy methods using nucleic acid molecules encoding the heavy and/or light immunoglobulin molecules that comprise the human anti-WNE antibodies. The invention also relates to transgenic animals or plants comprising nucleic acid molecules of the present invention.

### CROSS-REFERENCE TO RELATED APPLICATIONS

This application claims the benefit of the filing dates of U.S. Provisional Application Nos. 60/580,248, filed Jun. 15, 2004; 60/613,369, filed Sep. 27, 2004; and 60/646,839, filed Jan. 24, 2005; the disclosures of each of which are hereby incorporated by reference in their entirety.

### FUNDING

Work described herein was funded, in whole or in part, by National Institutes of Health grant R43 AI49646-01. The United States government may have rights in the invention.

### BACKGROUND OF THE INVENTION

West Nile virus is a member of the family Flaviviridae which also includes the Japanese encephalitis virus (JE), Tick-borne encephalitis virus (TBE), St. Louis Encephalitis virus (SLEV), Murray Valley encephalitis virus, dengue virus (including the four serotypes of: DEN-1, DEN-2, DEN-3, and DEN-4), and the family prototype, yellow fever virus (YF). Flavivirus infections are a global public health problem [C. G. Hayes, in The Arboviruses: Epidemiology and Ecology, T. P. Monathy, ed., CRC, Boca Raton, Fla., vol. 5, chap. 49 (1989); M. J. Cardosa, Br Med Bull, 54, pp. 395-405 (1998); Z. Hubalek and J. Halouzka, Emerg Infect Dis, 5, pp. 643-50 (1999)] with about half of the flaviviruses causing human diseases.

Flaviviruses are the most significant group of arthropod-transmitted viruses in terms of global morbidity and mortality. An estimated one hundred million cases of the most prevalent flaviviral disease, dengue fever, occur annually. Flaviviral disease typically occurs in the tropical and subtropical regions. Increased global population and urbanization coupled with the lack of sustained mosquito control measures, has distributed the mosquito vectors of flaviviruses throughout the tropics, subtropics, and some temperate areas. As a result, over half the world's population is at risk for flaviviral infection. Further, modern jet travel and human migration have raised the potential for global spread of these pathogens.

West Nile virus infections generally have mild symptoms, although infections can be fatal in elderly and immunocompromised patients. Typical symptoms of mild WN virus infections include fever, headache, body aches, rash and swollen lymph glands. Severe disease with encephalitis is typically found in elderly patients [D. S. Asnis et al., supra]. Death can result from effects on the central nervous system. Sixty-two severe cases and seven deaths were attributed to WN virus encephalitis during the 1999 outbreak [CDC, supra; CDC, supra; D. S. Asnis et al., supra]. Although most WN virus infections are mild, concern is particularly heightened by the potentially fatal outcome of this mosquito-transmitted disease.

Substantial morbidity has been observed following hospitalization for WNV infection. A study of patients in New York and New Jersey in 2000 found that more than half of patients did not return to their full functional level following discharge, only ⅓ were fully ambulatory (Campbell, G. L., Marfin, A. A., Lanciotti, R. S., and Gubler, D. J. 2002. Lancet Infect Dis 2:519-529), and only 28% of patients in one study returned home without additional support (Pepperell, C., Rau, N., Krajden, S., Kern, R., Humar, A., Mederski, B., Simor, A., Low, D. E., McGeer, A., Mazzulli, T., et al. 2003. CMAJ 168:1399-1405). Persistent symptoms reported in a one year follow up of 1999 New York patients include fatigue, memory loss, difficulty walking, muscle weakness, and depression (Petersen, L. R., Marfin, A. A., and Gubler, D. J. 2003. JAMA 290:524-528).

The WN virus, like other flaviviruses, is enveloped by host cell membrane and contains the three structural proteins capsid (C), membrane (M), and envelope (E). The E and M proteins are found on the surface of the virion where they are anchored in the membrane. Mature E is glycosylated, whereas M is not, although its precursor, prM, is a glycoprotein. In other flaviviruses, glycoprotein E is the largest structural protein and contains functional domains responsible for cell surface attachment and intraendosomal fusion activities. In some flaviviruses, E protein has been reported to be a major target of the host immune system during a natural infection.

The flavivirus genome is a single positive-stranded RNA of approximately 10,500 nucleotides containing short 5′ and 3′ untranslated regions, a single long open reading frame (ORF), a 5′ cap, and a nonpolyadenylated 3′ terminus. The ten gene products encoded by the single, long ORF are contained in a polyprotein organized in the order, C (capsid), prM/M (membrane), E (envelope), NS1 (nonstructural protein 1), NS2A, NS2B, NS3, NS4A, NS4B, and NS5 [T. J. Chambers et al., Ann Rev Microbiol, 44, pp. 649-88 (1990)].

The E protein of flaviviruses is responsible for membrane fusion and mediates binding to host cellular receptors (Monath, T. P. 1990. Flaviviruses. In Virology. B. N. Fields, and D. M. Knipe, editors. New York: Raven Press. 763-814). The crystal structure of the tick-borne encephalitis virus (TBEV), dengue virus, serotype 2, (DENV-2), and dengue virus, serotype 3, (DENV-3) envelope proteins have been solved at high resolution [Rey, F. A., Heinz, F. X., Mandl, C., Kunz, C., and Harrison, S. C. (1995) Nature 375:291-298; Modis, Y., Ogata, S., Clements, D., and Harrison, S. C. (2003) Proc Natl Acad Sci USA 100:6986-6991; Modis, Y., Ogata, S., Clements, D., and Harrison, S. C. (2005) J Virol 79:1223-1231; Kuhn, R. J., Zhang, W., Rossmann, M. G., Pletnev, S. V., Corver, J., Lenches, E., Jones, C. T., Mukhopadhyay, S., Chipman, P. R., Strauss, E. G., et al. (2002) Cell 108:717-725; Allison, S. L., Schalich, J., Stiasny, K., Mandl, C. W., Kunz, C., and Heinz, F. X. (1995) J Virol 69:695-700; Ferlenghi, I., Clarke, M., Ruttan, T., Allison, S. L., Schalich, J., Heinz, F. X., Harrison, S. C., Rey, F. A., and Fuller, S. D. (2001) Mol Cell 7:593-602].

The E protein is approximately 500 amino acids in length, and is folded into three structural and functional domains: I, II, and III. Domain I (DI) is the central structural domain, and is hypothesized to be the region involved in low-pH triggered conformational changes. Additionally, DI is the site of the single, flavivirus conserved, glycosylated asparagine. Domain II (DII), the dimerization domain, is involved in virus-mediated membrane fusion. Domain III (DIII) is the putative receptor binding domain (Modis, Y., Ogata, S., Clements, D., and Harrison, S. C. (2003) Proc Natl Acad Sci USA 100:6986-6991; Crill, W. D., and Roehrig, J. T. (2001) J Virol 75:7769-7773).

The entry of WNV into host cells is presumably mediated by binding of DIII to its receptor [Crill, W. D., and Roehrig, J. T. (2001) J Virol 75:7769-7773]. Although a specific receptor molecule has not been identified, several candidate receptors have been suggested.

While flaviviruses exhibit similar structural features and components, the individual viruses are significantly different at both the sequence and antigenic levels. Indeed, antigenic distinctions have been used to define four different serotypes within just the dengue virus subgroup of the flaviviruses. Infection of an individual with one dengue serotype does not provide long-term immunity against the other serotypes and secondary infections with heterologous serotypes are becoming increasingly prevalent as multiple serotypes co-circulate in a geographic area. Such secondary infections indicate that vaccination or prior infection with any one flavivirus may not provide generalized protection against other flaviviruses. Attempts to develop suitable vaccines, which have especially focused on the dengue viruses are ongoing [S. B. Halstead, Science, 239, pp. 476-81 (1988); W. E. Brandt, J Infect Disease, 162, pp. 577-83 (1990); T. J. Chambers et al., Ann Rev Microbiol, 44, pp. 649-88 (1990); C. W. Mandl et al., Virology, 63, pp. 564-71 (1989); and E. A. Henchal and J. R. Putnak, Clin Microbiol Rev, 3, pp. 376-96 (1990)].

Currently, the only treatments for WNV infection are supportive. In vitro studies have found ribavirin and interferon-alpha2b to be effective against the virus (Anderson, J. F., and Rahal, J. J. (2002) Emerg Infect Dis 8:107-108; Jordan, I., Briese, T., Fischer, N., Lau, J. Y., and Lipkin, W. I. (2000) J Infect Dis 182:1214-1217; Weiss, D., Carr, D., Kellachan, J., Tan, C., Phillips, M., Bresnitz, E., and Layton, M. (2001) Emerg Infect Dis 7:654-658] and several human case studies have found that alpha-interferon may improve the clinical outcome of WNV infection [Kalil, A. C., Devetten, M. P., Singh, S., Lesiak, B., Poage, D. P., Bargenquast, K., Fayad, P., and Freifeld, A. G. (2005) Clin Infect Dis 40:764-766; Sayao, A. L., Suchowersky, O., Al-Khathaami, A., Klassen, B., Katz, N. R., Sevick, R., Tilley, P., Fox, J., and Patry, D. (2004) Can J Neurol Sci 31:194-203].

Future outbreaks of WN virus in the United States are a new and important public health concern. West Nile virus has spread rapidly across the United States, and there is currently no approved human vaccine or therapy to prevent or treat West Nile virus infection. Accordingly, there is an urgent need for additional therapeutic molecules to treat flavivirus, including West Nile virus, infections.

### SUMMARY OF THE INVENTION

The present invention provides an isolated anti-West Nile virus E protein (“WNE”) or antigen-binding portion thereof. In a preferred aspect of the invention, the antibodies are protective.

Provided herein are single chain anti-WNE antibodies comprising a heavy chain and a light chain variable domain (scFv), traditional four chain antibodies, and antigen-binding portions of such antibodies.

The invention provides a composition comprising the heavy and/or light chain, the variable domains thereof, or antigen-binding portions thereof of an anti-WNE antibody, or nucleic acid molecules encoding an antibody, antibody chain or variable domain thereof of the invention and a pharmaceutically acceptable carrier. Compositions of the invention may further comprise another component, such as a therapeutic agent or a diagnostic agent. Diagnostic and therapeutic methods are also provided by the invention.

The invention further relates to an isolated cell line that produces an anti-WNE antibody or antigen-binding portion thereof.

The invention also provides nucleic acid molecules encoding the heavy and/or light chain of an anti-WNE antibody, the variable domains thereof or antigen-binding portions thereof.

The invention provides vectors and host cells comprising the nucleic acid molecules, as well as methods of recombinantly producing the polypeptides encoded by the nucleic acid molecules.

The invention further relates to non-human transgenic animals or plants that express the heavy and/or light chain, or antigen-binding portions thereof, of an anti-WNE antibody.

### BRIEF DESCRIPTION OF THE DRAWINGS

FIG. 1 shows an alignment of the amino acid sequences corresponding to the respective V_{H} and V_{L} domains of antibodies 11, 71, 73, 85, 15, 95, 84, 10, 69, 79, and 94 with a consensus amino acid sequence, which is encoded by more than 50% of the genes at a given position. Dots in the consensus sequence are shown where <50% of the genes are encoded by the same amino acid, and dots in each sequence represent the same amino acid as the consensus. Gaps are represented by dashes. The framework regions 1-4 (FW 1-4) and complementarity-determining regions 1-3 (CDR1-3) for V_{H} and V_{L}, as well as V_{H} and V_{L} gene family designations are also shown.

FIG. 2 is a graph depicting the results of an ELISA measuring binding of scFvs to rWNV-E.

FIG. 3 is a graph showing neutralization of DENV-2 by scFv-Fcs.

FIGS. 4A-4C are graphs depicting the survival of WNV-infected mice passively immunized with anti-WNE antibodies.

FIG. 5 is a graph showing the survival of mice passively immunized with scFv-Fcs before injection with WNV.

FIGS. 6A-6B are graphs depicting the survival of mice passively immunized with scFv-Fcs after injection with WNV.

FIG. 7 is a graph depicting the half-life of antibody 79 (scFv-Fc) in mouse serum.

FIG. 8 is a graph of the amount of antibody dependent enhancement of infection in cultivated human macrophages observed after immunization with scFv-Fcs.

FIG. 9 is a graph showing binding of scFv-Fcs to WNV E protein ectodomain, DI/DII, and DIII.

FIG. 10 is a graph depicting binding inhibition of WNV to Vero cells by scFv-Fcs.

FIG. 11 is a graph depicting binding inhibition of WNV to Vero cells by scFv-Fcs pre and post virus attachment.

FIG. 12 is a graph depicting binding of scFv-Fcs to selected WNV E 20-mer peptides

FIG. 13 shows an alignment showing the region of the E protein represented by peptide 29 (underlined) among various flaviviruses.

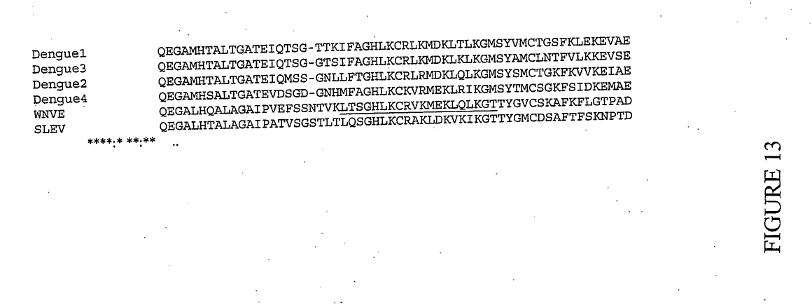

### DETAILED DESCRIPTION OF THE INVENTION

### Definitions and General Techniques

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art.

The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2nd ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The following terms, unless otherwise indicated, shall be understood to have the following meanings:

The term “polypeptide” encompasses native or artificial proteins, protein fragments and polypeptide analogs of a protein sequence. A polypeptide may be monomeric or polymeric.

The term “isolated protein”, “isolated polypeptide” or “isolated antibody” is a protein, polypeptide or antibody that by virtue of its origin or source of derivation (1) is not associated with naturally associated components that accompany it in its native state, (2) is free of other proteins from the same species, (3) is expressed by a cell from a different species, or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

Examples of isolated antibodies include an anti-WNE antibody that has been affinity purified using WNE or a fragment thereof, an anti-WNE antibody that has been synthesized by a hybridoma or other cell line in vitro, and a human anti-WNE antibody derived from a transgenic mouse.

A protein or polypeptide is “substantially pure,” “substantially homogeneous,” or “substantially purified” when at least about 60 to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “polypeptide fragment” as used herein refers to a polypeptide that has an amino-terminal and/or carboxy-terminal deletion, but where the remaining amino acid sequence is identical to the corresponding positions in the naturally-occurring sequence. In some embodiments, fragments are at least 5, 6, 8 or 10 amino acids long. In other embodiments, the fragments are at least 14, at least 20, at least 50, or at least 70, 80, 90, 100, 150 or 200 amino acids long.

The term “polypeptide analog” as used herein refers to a polypeptide that comprises a segment that has substantial identity to a portion of an amino acid sequence and that has at least one of the following properties: (1) specific binding to WNE under suitable binding conditions, (2) ability to treat, inhibit, or prevent a West Nile Virus infection, (3) ability to cross-react with different flaviviral E proteins. Typically, polypeptide analogs comprise a conservative amino acid substitution (or insertion or deletion) with respect to the native sequence. Analogs typically are at least 20 or 25 amino acids long, preferably at least 50, 60, 70, 80, 90, 100, 150 or 200 amino acids long or longer, and can often be as long as a full-length polypeptide. Some embodiments of the invention include polypeptide fragments or polypeptide analog antibodies with 1, 2, 3, 4, 5, 6, 7, 8, 9, 10, 11, 12, 13, 14, 15, 16 or 17 substitutions from the germline amino acid sequence.

In certain embodiments, amino acid substitutions to an anti-WNE antibody or antigen-binding portion thereof are those which: (1) reduce susceptibility to proteolysis, (2) reduce susceptibility to oxidation, (3) alter binding affinity for forming protein complexes, and (4) confer or modify other physicochemical or functional properties of such analogs, but still retain specific binding to WNE. Analogs can include various muteins of a sequence other than the normally-occurring peptide sequence. For example, single or multiple amino acid substitutions, preferably conservative amino acid substitutions, may be made in the normally-occurring sequence, preferably in the portion of the polypeptide outside the domain(s) forming intermolecular contacts. A conservative amino acid substitution should not substantially change the structural characteristics of the parent sequence; e.g., a replacement amino acid should not alter the anti-parallel β-sheet that makes up the immunoglobulin binding domain that occurs in the parent sequence, or disrupt other types of secondary structure that characterizes the parent sequence. In general, glycine and proline would not be used in an anti-parallel β-sheet. Examples of art-recognized polypeptide secondary and tertiary structures are described in Proteins, Structures and Molecular Principles (Creighton, Ed., W.H. Freeman and Company, New York (1984)); Introduction to Protein Structure (C. Branden and J. Tooze, eds., Garland Publishing, New York, N.Y. (1991)); and Thornton et al., Nature 354:105 (1991), incorporated herein by reference.

Non-peptide analogs are commonly used in the pharmaceutical industry as drugs with properties analogous to those of the template peptide. These types of non-peptide compounds are termed “peptide mimetics” or “peptidomimetics.” Fauchere, J. Adv. Drug Res. 15:29 (1986); Veber and Freidinger, TINS p. 392 (1985); and Evans et al., J. Med. Chem. 30:1229 (1987), incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a desired biochemical property or pharmacological activity), such as a human antibody, but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference); for example, by adding internal cysteine residues capable of forming intramolecular disulfide bridges which cyclize the peptide.

The term “antibody” refers to an intact immunoglobulin or to an antigen-binding portion thereof. For example, antibodies of the invention include nucleic acid and amino acid sequences encoded thereby of the scFvs 11, 71, 73, 85, 15, 95, 84, 10, 69, 79, and 94 described herein. An antigen-binding portion competes with the intact antibody for specific binding. See generally, Fundamental Immunology, Ch. 7 (Paul, W., ed., 2nd ed. Raven Press, N.Y. (1989)) (incorporated by reference in its entirety for all purposes). Antigen-binding portions may be produced by recombinant DNA techniques or by enzymatic or chemical cleavage of intact antibodies. In some embodiments, antigen-binding portions include Fab, Fab′, F(ab′)₂, Fd, Fv, dAb, and complementarity determining region (CDR) fragments, single-chain antibodies (scFv), chimeric antibodies, diabodies and polypeptides that contain at least a portion of an antibody that is sufficient to confer specific antigen binding to the polypeptide.

As used herein, a “protective antibody” is an antibody that confers protection, for some period of time, against any one of the physiological disorders associated with infection by a flavivirus in the Japanese Encephalitis Antgenic Complex, particularly by a West Nile Virus.

From N-terminus to C-terminus, both the mature light and heavy chain variable domains comprise the regions FR1, CDR1, FR2, CDR2, FR3, CDR3 and FR4. The assignment of amino acids to each domain herein is in accordance with the definitions of Kabat, Sequences of Proteins of Immunological Interest (National Institutes of Health, Bethesda, Md. (1987 and 1991)), Chothia & Lesk, J. Mol. Biol. 196:901-917 (1987) or Chothia et al., Nature 342:878-883 (1989).

As used herein, an Fd fragment means an antibody fragment that consists of the V_{H} and C_{H}1 domains; an Fv fragment consists of the V_{L} and V_{H} domains of a single arm of an antibody; and a dAb fragment (Ward et al., Nature 341:544-546 (1989)) consists of a V_{H} domain.

In some embodiments, the antibody is a single-chain antibody (scFv) in which a V_{L} and a V_{H} domain are paired to form a monovalent molecule via a synthetic linker that enables them to be made as a single protein chain. (Bird et al., Science 242:423-426 (1988) and Huston et al., Proc. Natl. Acad. Sci. USA 85:5879-5883 (1988).) In some embodiments, the antibodies are diabodies, i.e., are bivalent antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites. (See e.g., Holliger P. et al., Proc. Natl. Acad. Sci. USA 90:6444-6448 (1993), and Poljak R. J. et al., Structure 2:1121-1123 (1994).) In some embodiments, one or more CDRs from an antibody of the invention may be incorporated into a molecule either covalently or noncovalently to make it an immunoadhesin that specifically binds to WNE. In such embodiments, the CDR(s) may be incorporated as part of a larger polypeptide chain, may be covalently linked to another polypeptide chain, or may be incorporated noncovalently.

In embodiments having one or more binding sites, the binding sites may be identical to one another or may be different.

As used herein, the term “human antibody” means any antibody in which the variable and constant domain sequences are human sequences. The term encompasses antibodies with sequences derived from human genes, but which have been changed, e.g., to decrease possible immunogenicity, increase affinity, eliminate cysteines that might cause undesirable folding, etc. The term encompasses such antibodies produced recombinantly in non-human cells, which might impart glycosylation not typical of human cells. These antibodies may be prepared in a variety of ways, as described below.

The term “chimeric antibody” as used herein means an antibody that comprises regions from two or more different antibodies. In one embodiment, one or more of the CDRs of the chimeric antibody are derived from a human anti-WNE antibody. In another embodiment, all of the CDRs are derived from human anti-WNE antibodies. In another embodiment, the CDRs from more than one human anti-WNE antibodies are combined in a chimeric antibody. For instance, a chimeric antibody may comprise a CDR1 from the light chain of a first human anti-WNE antibody, a CDR2 from the light chain of a second human anti-WNE antibody and a CDR3 from the light chain of a third human anti-WNE antibody, and CDRs from the heavy chain may be derived from one or more other anti-WNE antibodies. Further, the framework regions may be derived from one of the anti-WNE antibodies from which one or more of the CDRs are taken or from one or more different human antibodies.

In some embodiments, a chimeric antibody of the invention is a humanized anti-WNE antibody. A humanized anti-WNE antibody of the invention comprises the amino acid sequence of one or more framework regions and/or the amino acid sequence from at least a portion of the constant region of one or more human anti-WNE antibodies of the invention and CDRs derived from a non-human anti-WNE antibody.

Fragments or analogs of antibodies or immunoglobulin molecules can be readily prepared by those of ordinary skill in the art following the teachings of this specification. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. See Bowie et al., Science 253:164 (1991).

The term “surface plasmon resonance”, as used herein, refers to an optical phenomenon that allows for the analysis of real-time biospecific interactions by detection of alterations in protein concentrations within a biosensor matrix, for example using the BIACORE™ system (Pharmacia Biosensor AB, Uppsala, Sweden and Piscataway, N.J.). For further descriptions, see Jonsson U. et al., Ann. Biol. Clin. 51:19-26 (1993); Jonsson U. et al., Biotechniques 11:620-627 (1991); Jonsson B. et al., J. Mol. Recognit. 8:125-131 (1995); and Johnsson B. et al., Anal. Biochem. 198:268-277 (1991).

The term “K_{D}” refers to the equilibrium dissociation constant of a particular antibody-antigen interaction.

The term “epitope” includes any protein determinant capable of specific binding to an immunoglobulin or T-cell receptor or otherwise interacting with a molecule. Epitopic determinants generally consist of chemically active surface groupings of molecules such as amino acids or carbohydrate or sugar side chains and generally have specific three dimensional structural characteristics, as well as specific charge characteristics. An epitope may be “linear” or “conformational.” In a linear epitope, all of the points of interaction between the protein and the interacting molecule (such as an antibody) occur linearly along the primary amino acid sequence of the protein. In a conformational epitope, the points of interaction occur across amino acid residues on the protein that are separated from one another. An antibody is said to specifically bind an antigen when the dissociation constant is ≦1 mM, preferably ≦100 nM and most preferably ≦10 nM. In certain embodiments, the K_{D} is 1 pM to 500 μM. In other embodiments, the K_{D} is between 500 pM to 1 μM. In other embodiments, the K_{D} is between 1 μM to 100 nM. In other embodiments, the K_{D} is between 100 nM to 10 mM. Once a desired epitope on an antigen is determined, it is possible to generate antibodies to that epitope, e.g., using the techniques described in the present invention. Alternatively, during the discovery process, the generation and characterization of antibodies may elucidate information about desirable epitopes. From this information, it is then possible to competitively screen antibodies for binding to the same epitope. An approach to achieve this is to conduct cross-competition studies to find antibodies that competitively bind with one another, e.g., the antibodies compete for binding to the antigen. A high throughput process for “binning” antibodies based upon their cross-competition is described in International Patent Application No. WO 03/48731.

As used herein, a “protective epitope” is (1) an epitope that is recognized by a protective antibody, and/or (2) an epitope that, when used to immunize an animal, elicits an immune response sufficient to prevent or lessen the severity for some period of time, of infection by a flavivirus in the Japanese Encephalitis Antgenic Complex, particularly by a West Nile Virus. Again, preventing or lessening the severity of infection may be evidenced by an amelioration in any of the physiological manifestations of such an infection. It also may be evidenced by a decrease in the level of viral particles in the treated animal or a decrease in the number of viruses that can be cultured from a biological sample from an infected animal. A protective epitope may comprise a T cell epitope, a B cell epitope, or combinations thereof.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), incorporated herein by reference.

The term “polynucleotide” as referred to herein means a polymeric form of nucleotides of at least 10 bases in length, either ribonucleotides or deoxynucleotides or a modified form of either type of nucleotide. The term includes single and double stranded forms.

The term “isolated polynucleotide” as used herein means a polynucleotide of genomic, cDNA, or synthetic origin or some combination thereof, which by virtue of its origin the “isolated polynucleotide” (1) is not associated with all or a portion of a polynucleotides with which the “isolated polynucleotide” is found in nature, (2) is operably linked to a polynucleotide to which it is not linked in nature, or (3) does not occur in nature as part of a larger sequence.

The term “naturally occurring nucleotides” as used herein includes deoxyribonucleotides and ribonucleotides. The term “modified nucleotides” as used herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al., Nucl. Acids Res. 14:9081 (1986); Stec et al., J. Am. Chem. Soc. 106:6077 (1984); Stein et al., Nucl. Acids Res. 16:3209 (1988); Zon et al., Anti-Cancer Drug Design 6:539 (1991); Zon et al., Oligonucleotides and Analogues: A Practical Approach, pp. 87-108 (F. Eckstein, Ed., Oxford University Press, Oxford England (1991)); U.S. Pat. No. 5,151,510; Uhlmann and Peyman, Chemical Reviews 90.543 (1990), the disclosures of which are hereby incorporated by reference. An oligonucleotide can include a label for detection, if desired.

“Operably linked” sequences include both expression control sequences that are contiguous with the gene of interest and expression control sequences that act in trans or at a distance to control the gene of interest. The term “expression control sequence” as used herein means polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “vector”, as used herein, means a nucleic acid molecule capable of transporting another nucleic acid to which it has been linked. In some embodiments, the vector is a plasmid, i.e., a circular double stranded piece of DNA into which additional DNA segments may be ligated. In some embodiments, the vector is a viral vector, wherein additional DNA segments may be ligated into the viral genome. In some embodiments, the vectors are capable of autonomous replication in a host cell into which they are introduced (e.g., bacterial vectors having a bacterial origin of replication and episomal mammalian vectors). In other embodiments, the vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”).

The term “recombinant host cell” (or simply “host cell”), as used herein, means a cell into which a recombinant expression vector has been introduced. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. “High stringency” or “highly stringent” conditions can be used to achieve selective hybridization conditions as known in the art and discussed herein. One example of “high stringency” or “highly stringent” conditions is the incubation of a polynucleotide with another polynucleotide, wherein one polynucleotide may be affixed to a solid surface such as a membrane, in a hybridization buffer of 6×SSPE or SSC, 50% formamide, 5×Denhardt's reagent, 0.5% SDS, 100 μg/ml denatured, fragmented salmon sperm DNA at a hybridization temperature of 42° C. for 12-16 hours, followed by twice washing at 55° C. using a wash buffer of 1×SSC, 0.5% SDS. See also Sambrook et al., supra, pp. 9.50-9.55.

The term “percent sequence identity” in the context of nucleic acid sequences means the residues in two sequences that are the same when aligned for maximum correspondence. The length of sequence identity comparison may be over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson, Methods Enzymol. 183:63-98 (1990); Pearson, Methods Mol. Biol. 132:185-219 (2000); Pearson, Methods Enzymol. 266:227-258 (1996); Pearson, J. Mol. Biol. 276:71-84 (1998); incorporated herein by reference). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, incorporated herein by reference.

A reference to a nucleotide sequence encompasses its complement unless otherwise specified. Thus, a reference to a nucleic acid having a particular sequence should be understood to encompass its complementary strand, with its complementary sequence.

As used herein, the terms “percent sequence identity” and “percent sequence homology” are used interchangeably.

The term “substantial similarity” or “substantial sequence similarity,” when referring to a nucleic acid or fragment thereof, means that when optimally aligned with appropriate nucleotide insertions or deletions with another nucleic acid (or its complementary strand), there is nucleotide sequence identity in at least about 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

As applied to polypeptides, the term “substantial identity” means that two peptide sequences, when optimally aligned, such as by the programs GAP or BESTFIT using default gap weights as supplied with the programs, share at least 70%, 75% or 80% sequence identity, preferably at least 90% or 95% sequence identity, and more preferably at least 97%, 98% or 99% sequence identity. In certain embodiments, residue positions that are not identical differ by conservative amino acid substitutions. A “conservative amino acid substitution” is one in which an amino acid residue is substituted by another amino acid residue having a side chain R group with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity may be adjusted upwards to correct for the conservative nature of the substitution. Means for making this adjustment are well-known to those of skill in the art. See, e.g., Pearson, Methods Mol. Biol. 243:307-31 (1994). Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al., Science 256:1443-45 (1992), incorporated herein by reference. A “moderately conservative” replacement is any change having a nonnegative value in the PAM250 log-likelihood matrix.

Sequence identity for polypeptides is typically measured using sequence analysis software. Protein analysis software matches sequences using measures of similarity assigned to various substitutions, deletions and other modifications, including conservative amino acid substitutions. For instance, GCG contains programs such as “Gap” and “Bestfit” which can be used with default parameters as specified by the programs to determine sequence homology or sequence identity between closely related polypeptides, such as homologous polypeptides from different species of organisms or between a wild type protein and a mutein thereof. See, e.g., GCG Version 6.1 (University of Wisconsin, Wis.). Polypeptide sequences also can be compared using FASTA using default or recommended parameters, see GCG Version 6.1. FASTA (e.g., FASTA2 and FASTA3) provides alignments and percent sequence identity of the regions of the best overlap between the query and search sequences (Pearson, Methods Enzymol. 183:63-98 (1990); Pearson, Methods Mol. Biol. 132:185-219 (2000)). Another preferred algorithm when comparing a sequence of the invention to a database containing a large number of sequences from different organisms is the computer program BLAST, especially blastp or tblastn, using default parameters as supplied with the programs. See, e.g., Altschul et al., J. Mol. Biol. 215:403-410 (1990); Altschul et al., Nucleic Acids Res. 25:3389-402 (1997).

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences.

As used herein, the terms “label” or “labeled” refers to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

Throughout this specification and claims, the word “comprise,” or variations such as “comprises” or “comprising,” will be understood to imply the inclusion of a stated integer or group of integers but not the exclusion of any other integer or group of integers.

### Human Anti-WNE Antibodies and Characterization Thereof

In one embodiment, the invention provides anti-WNE antibodies. In some embodiments, the antibodies are human. In another embodiment, the invention provides humanized anti-WNE antibodies. In some embodiments, human anti-WNE antibodies are produced by immunizing a non-human transgenic animal, e.g., a rodent, whose genome comprises human immunoglobulin genes so that the transgenic animal produces human antibodies.

An anti-WNE antibody of the invention can comprise a human kappa or a human lambda light chain or an amino acid sequence derived therefrom.

In some embodiments, the light chain of the human anti-WNE antibody comprises the V_{L} amino acid sequence of antibody 11, 71, 73, 85, 15, 95, 84, 10, 69, 79, or 94 or said amino acid sequence having up to 1, 2, 3, 4, 5, 6, 7, 8, 9, or 10 conservative amino acid substitutions and/or a total of up to 3 non-conservative amino acid substitutions.

In certain embodiments, the light chain of the anti-WNE antibody comprises the light chain CDR1, CDR2 and CDR3 amino acid sequences of an antibody selected from antibody 11, 71, 73, 85, 15, 95, 84, 10, 69, 79, or 94 or said CDR regions each having less than 4 or less than 3 conservative amino acid substitutions and/or a total of three or fewer non-conservative amino acid substitutions.

In some embodiments, the heavy chain comprises the V_{H} amino acid sequence of antibody 11, 71, 73, 85, 15, 95, 84, 10, 69, 79, or 94; or said V_{H} amino acid sequence having up to 1, 2, 3, 4, 6, 8, or 10 conservative amino acid substitutions and/or a total of up to 3 non-conservative amino acid substitutions.

In some embodiments, the heavy chain comprises the heavy chain CDR1, CDR2 and CDR3 regions of antibody 11, 71, 73, 85, 15, 95, 84, 10, 69, 79, or 94; or said CDR regions each having less than 8, less than 6, less than 4, or less than 3 conservative amino acid substitutions and/or a total of three or fewer non-conservative amino acid substitutions.

In another embodiment, the antibody comprises a light chain as disclosed above and a heavy chain as disclosed above. In a further embodiment, the light chain CDRs and the heavy chain CDRs are from the same antibody.

One type of amino acid substitution that may be made is to change one or more cysteines in the antibody, which may be chemically reactive, to another residue, such as, without limitation, alanine or serine. In one embodiment, there is a substitution of a non-canonical cysteine. The substitution can be made in a CDR or framework region of a variable domain or in the constant domain of an antibody. In some embodiments, the cysteine is canonical.

Another type of amino acid substitution that may be made is to change any potential proteolytic sites in the antibody. Such sites may occur in a CDR or framework region of a variable domain or in the constant domain of an antibody. Substitution of cysteine residues and removal of proteolytic sites may decrease the risk of any heterogeneity in the antibody product and thus increase its homogeneity. Another type of amino acid substitution is to eliminate asparagine-glycine pairs, which form potential deamidation sites, by altering one or both of the residues.

In one aspect, the invention relates to eleven human anti-WNE antibodies that are scFvs. Table 1 lists the sequence identifiers (SEQ ID NOS:) of the nucleic acids encoding the variable domains of the heavy and light chains, and the corresponding deduced amino acid sequences.

TABLE 1
HUMAN ANTI-WNE ANTIBODIES

| SEQUENCE IDENTIFIER | |
|---|---|
| (SEQ ID NO:) | |
| Variable Domains | |
| Heavy | Light |

| scFv | DNA | Protein | DNA | Protein |
|---|---|---|---|---|
| 11 | 45 | 23 | 56 | 34 |
| 71 | 46 | 24 | 57 | 35 |
| 73 | 47 | 25 | 58 | 36 |
| 85 | 48 | 26 | 59 | 37 |
| 15 | 49 | 27 | 60 | 38 |
| 95 | 50 | 28 | 61 | 39 |
| 84 | 51 | 29 | 62 | 40 |
| 10 | 52 | 30 | 63 | 41 |
| 69 | 53 | 31 | 64 | 42 |
| 79 | 54 | 32 | 65 | 43 |
| 94 | 55 | 33 | 66 | 44 |

In still further embodiments, the invention includes antibodies comprising variable domain amino acid sequences with more than 80%, more than 85%, more than 90%, more than 95%, more than 96%, more than 97%, more than 98% or more than 99% sequence identity to a variable domain amino acid sequence of any of the above-listed human anti-WNE antibodies (e.g., antibodies 11, 71, 73, 85, 15, 95, 84, 10, 69, 79, or 94).

### Class and Subclass of Anti-WNE Antibodies

The class and subclass of anti-WNE antibodies may be determined by any method known in the art. In general, the class and subclass of an antibody may be determined using antibodies that are specific for a particular class and subclass of antibody. Such antibodies are commercially available. The class and subclass can be determined by ELISA, or Western Blot as well as other techniques. Alternatively, the class and subclass may be determined by sequencing all or a portion of the constant domains of the heavy and/or light chains of the antibodies, comparing their amino acid sequences to the known amino acid sequences of various class and subclasses of immunoglobulins, and determining the class and subclass of the antibodies.

In some embodiments, the anti-WNE antibody is a monoclonal antibody. The anti-WNE antibody can be an IgG, an IgM, an IgE, an IgA, or an IgD molecule. In a preferred embodiment, the anti-WNE antibody is an IgG and is an IgG1, IgG2, IgG3, or IgG4 subclass.

### Binding Affinity of Anti-WNE Antibodies to WNE

In some embodiments of the invention, the anti-WNE antibodies bind to WNE with high affinity. In some embodiments, the anti-WNE antibody binds to WNE with a K_{D} of 6×10⁻⁸ M or less. In other preferred embodiments, the antibody binds to WNE with a K_{D} of 2×10⁻⁸ M, 2×10⁻⁹ M, or 1×10⁻¹⁰ M, 4×10⁻¹¹ M or 2×10⁻¹¹ M or less. In an even more preferred embodiment, the antibody binds to WNE with substantially the same K_{D} as an antibody selected from 11, 71, 73, 85, 15, 95, 84, 10, 69, 79, or 94. In still another preferred embodiment, the antibody binds to WNE with substantially the same K_{D} as an antibody that comprises a heavy chain variable domain having the amino acid sequence of a V_{H} domain selected from SEQ ID NOS: 23-33, a light chain variable domain having the amino acid sequence of a V_{L} domain selected from SEQ ID NOS: 34-44 or both. In another preferred embodiment, the antibody binds to WNE with substantially the same K_{D} as an antibody that comprises the CDR regions of a light chain variable domain having the amino acid sequence of a V_{L} domain of any of SEQ ID NOS: 34-44 or that comprises the CDR regions of a heavy chain variable domain having the amino acid sequence a V_{H} domain of any of SEQ ID NOS: 23-33.

In some embodiments, the anti-WNE antibody has a low dissociation rate constant (k_{off}). In some embodiments, the anti-WNE antibody has a k_{off} of 7.0×10⁻³ s⁻¹ or lower or a k_{off} of 7.0×10⁻⁴ s⁻¹ or lower or a k_{off} of 4.0×10⁻⁷ s⁻¹. In other preferred embodiments, the antibody binds to WNE with a k_{off} of 1×10⁻⁵ s⁻¹ or lower. In some embodiments, the k_{off} is substantially the same as an antibody described herein, including an antibody selected from 11, 71, 73, 85, 15, 95, 84, 10, 69, 79, or 94. In some embodiments, the antibody binds to WNE with substantially the same k_{off} as an antibody that comprises the CDR regions of a heavy chain or the CDR regions of a light chain from an antibody selected from 11, 71, 73, 85, 15, 95, 84, 10, 69, 79, or 94. In some embodiments, the antibody binds to WNE with substantially the same k_{off} as an antibody that comprises a heavy chain variable domain having the amino acid sequence of a V_{H} domain of any of SEQ ID NOS: 23-33, a light chain variable domain having the amino acid sequence of a V_{L} domain of any of SEQ ID NOS: 34-44 or both. In another preferred embodiment, the antibody binds to WNE with substantially the same k_{off} as an antibody that comprises the CDR regions of a light chain variable domain having the amino acid sequence of a V_{L} domain of any of SEQ ID NOS: 34-44; or the CDR regions of a heavy chain variable domain having the amino acid sequence of a V_{H} domain of any of SEQ ID NOS: 23-33.

The binding affinity and dissociation rate of an anti-WNE antibody to WNE can be determined by methods known in the art. The binding affinity can be measured by ELISAs, RIAs, flow cytometry, or surface plasmon resonance, such as BIACORE™. The dissociation rate can be measured by surface plasmon resonance. Preferably, the binding affinity and dissociation rate is measured by surface plasmon resonance. More preferably, the binding affinity and dissociation rate are measured using BIACORE™. One can determine whether an antibody has substantially the same K_{D} as an anti-WNE antibody by using methods known in the art. Example III exemplifies a method for determining affinity constants of anti-WNE antibodies by BIACORE™.

### Identification of WNE Epitopes Recognized by Anti-WNE Antibodies

The invention provides a human anti-WNE antibody that binds to WNE and competes or cross-competes with and/or binds the same epitope as: (a) an antibody selected from antibodies 11, 71, 73, 85, 15, 95, 84, 10, 69, 79, or 94; (b) an antibody that comprises a heavy chain variable domain having an amino acid sequence selected from the group consisting of SEQ ID NOS: 23-33, (c) an antibody that comprises a light chain variable domain having an amino acid sequence selected from the group consisting of SEQ ID NOS: 34-44, or (d) an antibody that comprises both a heavy chain variable domain as defined in (b) and a light chain variable domain as defined in (c).

One can determine whether an antibody binds to the same epitope or competes for binding with an anti-WNE antibody by using methods known in the art. In one embodiment, one allows a reference anti-WNE antibody to bind to WNE or a portion thereof under saturating conditions and then measures the ability of a test antibody to bind to WNE. If the test antibody is able to bind to WNE at the same time as the reference anti-WNE antibody, then the test antibody binds to a different epitope than the anti-WNE antibody. However, if the test antibody is not able to bind to WNE at the same time, then the test antibody binds to the same epitope, an overlapping epitope, or an epitope that is in close proximity to the epitope bound by the reference anti-WNE antibody. This experiment can be performed using ELISA, RIA, BIACORE™, or flow cytometry. In a preferred embodiment, the experiment is performed using ELISA. Methods of determining K_{D} are discussed further below.

To determine whether an antibody cross-competes with a reference anti-WNE antibody, one conducts the above-described test in two directions. That is, one tests the ability of the test antibody to bind WNE in the presence of the reference antibody and vice versa.

### Methods of Producing Antibodies and Antibody Producing Cell Lines

### Immunization

In some embodiments, human antibodies are produced by immunizing a non-human, transgenic animal comprising within its genome some or all of human immunoglobulin heavy chain and light chain loci with a WNE antigen. In certain embodiments, the transgenic animal is a mouse, such as a mouse to comprise large fragments of human immunoglobulin heavy chain and light chain loci and deficient in mouse antibody production. See, e.g., Green et al., Nature Genetics 7:13-21 (1994) and U.S. Pat. Nos. 5,916,771, 5,939,598, 5,985,615, 5,998,209, 6,075,181, 6,091,001, 6,114,598, 6,130,364, 6,162,963 and 6,150,584.

In another aspect, the invention provides a method for making anti-WNE antibodies from non-human, non-mouse animals by immunizing non-human transgenic animals that comprise human immunoglobulin loci with a WNE antigen. One can produce such animals using the methods described in the above-cited documents. The methods disclosed in these documents can be modified as described in U.S. Pat. No. 5,994,619, which is hereby incorporated by reference. U.S. Pat. No. 5,994,619 describes methods for producing novel cultured inner cell mass (CICM) cells and cell lines, derived from pigs and cows, and transgenic CICM cells into which heterologous DNA has been inserted. CICM transgenic cells can be used to produce cloned transgenic embryos, fetuses, and offspring. The '619 patent also describes methods of producing transgenic animals that are capable of transmitting the heterologous DNA to their progeny. In preferred embodiments of the current invention, the non-human animals are mammals, particularly rats, sheep, pigs, goats, cattle or horses.

In some embodiments, the non-human animal comprising human immunoglobulin genes are animals that have a human immunoglobulin “minilocus”. In the minilocus approach, an exogenous Ig locus is mimicked through the inclusion of individual genes from the Ig locus. Thus, one or more V_{H} genes, one or more D_{H} genes, one or more J_{H} genes, a mu constant domain, and a second constant domain (preferably a gamma constant domain) are formed into a construct for insertion into an animal. This approach is described, inter alia, in U.S. Pat. Nos. 5,545,807, 5,545,806, 5,569,825, 5,625,126, 5,633,425, 5,661,016, 5,770,429, 5,789,650, 5,814,318, 5,591,669, 5,612,205, 5,721,367, 5,789,215, and 5,643,763, hereby incorporated by reference.

In another aspect, the invention provides a method for making humanized anti-WNE antibodies. In some embodiments, non-human animals are immunized with a WNE antigen as described below under conditions that permit antibody production. Antibody-producing cells are isolated from the animals, fused with myelomas to produce hybridomas, and nucleic acids encoding the heavy and light chains of an anti-WNE antibody of interest are isolated. These nucleic acids are subsequently engineered using techniques known to those of skill in the art and as described further below to reduce the amount of non-human sequence, i.e., to humanize the antibody to reduce the immune response in humans

In some embodiments, the WNE antigen is isolated and/or purified WNE. In some embodiments, the WNE antigen is a fragment of WNE. In some embodiments, the WNE fragment is the ectodomain of WNE. In some embodiments, the WNE fragment is DI/DII. In some embodiments, the WNE fragment is DI/DIII. In some embodiments, the WNE fragment comprises at least one epitope of WNE. In other embodiments, the WNE antigen is a cell that expresses or overexpresses WNE or an immunogenic fragment thereof on its surface. In some embodiments, the WNE antigen is a WNE fusion protein. In some embodiments, the WNE is a synthetic peptide immunogen.

Immunization of animals can be by any method known in the art. See, e.g., Harlow and Lane, Antibodies: A Laboratory Manual, New York: Cold Spring Harbor Press, 1990. Methods for immunizing non-human animals such as mice, rats, sheep, goats, pigs, cattle and horses are well known in the art. See, e.g., Harlow and Lane, supra, and U.S. Pat. No. 5,994,619. In a preferred embodiment, the WNE antigen is administered with an adjuvant to stimulate the immune response. Exemplary adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. In some embodiments, if a polypeptide is being administered, the immunization schedule will involve two or more administrations of the polypeptide, spread out over several weeks.

### Production of Antibodies and Antibody-Producing Cell Lilies

In one embodiment, phage display techniques, as described herein, can be used to provide libraries containing a repertoire of antibodies with varying affinities for WNE. For production of such repertoires, it is unnecessary to immortalize the B cells from the immunized animal. Rather, the primary B cells can be used directly as a source of DNA. The mixture of cDNAs obtained from B cell, e.g., derived from spleens, is used to prepare an expression library, for example, a phage display library transfected into E. coli. The resulting cells are tested for immunoreactivity to WNE. Techniques for the identification of high affinity human antibodies from such libraries are described by Griffiths et al., EMBO J., 13:3245-3260 (1994); Nissim et al., ibid, pp. 692-698 and by Griffiths et al., ibid, 12:725-734, which are incorporated by reference. Ultimately, clones from the library are identified that produce binding affinities of a desired magnitude for the antigen and the DNA encoding the product responsible for such binding is recovered and manipulated for standard recombinant expression. Phage display libraries may also be constructed using previously manipulated nucleotide sequences and screened in a similar fashion. In general, the cDNAs encoding heavy and light chains are independently supplied or linked to form Fv analogs for production in the phage library.

The phage library is then screened for the antibodies with the highest affinities for WNE and the genetic material recovered from the appropriate clone. Further rounds of screening can increase affinity of the original antibody isolated.

In other embodiments, after immunization of an animal with a WNE antigen, antibodies and/or antibody-producing cells can be obtained from the animal. In some embodiments, anti-WNE antibody-containing serum is obtained from the animal by bleeding or sacrificing the animal. The serum may be used as it is obtained from the animal, an immunoglobulin fraction may be obtained from the serum, or the anti-WNE antibodies may be purified from the serum.

In some embodiments, antibody-producing immortalized cell lines are prepared from cells isolated from the immunized animal. After immunization, the animal is sacrificed and lymph node and/or splenic B cells are immortalized by any means known in the art. Methods of immortalizing cells include, but are not limited to, transfecting them with oncogenes, infecting them with an oncogenic virus and cultivating them under conditions that select for immortalized cells, subjecting them to carcinogenic or mutating compounds, fusing them with an immortalized cell, e.g., a myeloma cell, and inactivating a tumor suppressor gene. See, e.g., Harlow and Lane, supra. If fusion with myeloma cells is used, the myeloma cells preferably do not secrete immunoglobulin polypeptides (a non-secretory cell line). Immortalized cells are screened using WNE, a portion thereof, or a cell expressing WNE. In a preferred embodiment, the initial screening is performed using an enzyme-linked immunoassay (ELISA) or a radioimmunoassay. An example of ELISA screening is provided in WO 00/37504, incorporated herein by reference.

Anti-WNE antibody-producing cells, e.g., hybridomas, are selected, cloned and further screened for desirable characteristics, including robust growth, high antibody production and desirable antibody characteristics, as discussed further below. Hybridomas can be expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, the immunized animal is a non-human animal that expresses human immunoglobulin genes and the splenic B cells are fused to a myeloma cell line from the same species as the non-human animal.

Thus, in one embodiment, the invention provides methods for producing a cell line that produces a human monoclonal antibody or a fragment thereof directed to WNE comprising (a) immunizing a non-human transgenic animal described herein with WNE, a portion of WNE or a cell or tissue expressing WNE; (b) allowing the transgenic animal to mount an immune response to WNE; (c) isolating antibody-producing cells from transgenic animal; (d) immortalizing the antibody-producing cells; (e) creating individual monoclonal populations of the immortalized antibody-producing cells; and (f) screening the immortalized antibody-producing cells to identify an antibody directed to WNE.

In another aspect, the invention relates to hybridomas that produce a human anti-WNE antibody. In some embodiments, the hybridomas are mouse hybridomas, as described above. In other embodiments, the hybridomas are produced in a non-human, non-mouse species such as rats, sheep, pigs, goats, cattle or horses. In another embodiment, the hybridomas are human hybridomas.

In one embodiment of the invention, antibody-producing cells are isolated and expressed in a host cell, for example myeloma cells. In another preferred embodiment, a transgenic animal is immunized with WNE, primary cells, e.g., spleen or peripheral blood cells, are isolated from an immunized transgenic animal and individual cells producing antibodies specific for the desired antigen are identified. Polyadenylated mRNA from each individual cell is isolated and reverse transcription polymerase chain reaction (RT-PCR) is performed using sense primers that anneal to variable domain sequences, e.g., degenerate primers that recognize most or all of the FR1 regions of human heavy and light chain variable region genes and anti-sense primers that anneal to constant or joining region sequences. cDNAs of the heavy and light chain variable domains are then cloned and expressed in any suitable host cell, e.g., a myeloma cell, as chimeric antibodies with respective immunoglobulin constant regions, such as the heavy chain and K or λ constant domains. See Babcook, J. S. et al., Proc. Natl. Acad. Sci. USA 93:7843-48, 1996, incorporated herein by reference. Anti WNE antibodies may then be identified and isolated as described herein.

### Nucleic Acids, Vectors, Host Cells, and Recombinant Methods of Making Antibodies

### Nucleic Acids

The present invention also encompasses nucleic acid molecules encoding anti-WNE antibodies. In some embodiments, different nucleic acid molecules encode a heavy chain and a light chain of an anti-WNE immunoglobulin. In other embodiments, the same nucleic acid molecule encodes a heavy chain and a light chain of an anti-WNE immunoglobulin.

In some embodiments, the nucleic acid molecule encoding the variable domain of the light chain (V_{L}) comprises a human V lambda 1 family gene, a human V lambda 2 family gene, a human V lambda 3 family gene, or a human V lambda 8 family gene.

In some embodiments, the nucleic acid molecule comprises a nucleotide sequence that encodes the V_{L} amino acid sequence of antibody 11, 71, 73, 85, 15, 95, 84, 10, 69, 79, or 94, or said sequence with 1, 2, 3, 4, 5, 6, 7, 8, 9, or 10 amino acid substitutions. In some embodiments, the nucleic acid encodes an amino acid sequence comprising the light chain CDRs of one of said above-listed antibodies.

In some embodiments, the nucleic acid molecule comprises a nucleotide sequence that encodes an amino acid sequence selected from the group consisting of SEQ ID NOS: 12-22. In some preferred embodiments, the nucleic acid molecule comprises a nucleotide sequence selected from the group consisting of SEQ ID NOS: 1-11, or a portion thereof.

In some embodiments, the nucleic acid encodes the amino acid sequence of the light chain CDRs of said antibody.

In some embodiments, the nucleic acid molecule encodes a V_{L} amino acid sequence that is at least 70%, 75%, 80%, 85%, 90%, 95%, 97%, 98% or 99% identical to a V_{L} amino acid sequence of any one of a V_{L} domain of antibodies 11, 71, 73, 85, 15, 95, 84, 10, 69, 79, or 94, or an amino acid sequence of a V_{L} domain as depicted in of any one of SEQ ID NOS: 34-44. Nucleic acid molecules of the invention include nucleic acids that hybridize under highly stringent conditions, such as those described above, to a nucleic acid molecule encoding the amino acid sequence of a V_{L} domain depicted in any one of SEQ ID NOS: 34-44, or to a nucleic acid molecule depicted in SEQ ID NOS: 56-66.

In another embodiment, the nucleic acid encodes a full-length light chain or a light chain comprising an amino acid sequence selected from the group consisting of SEQ ID NOS: 33-44, or any one of said amino acid sequences comprising a mutation. In some embodiments, the nucleic acid may comprise the nucleotide sequence of any of SEQ ID NOS: 56-66, or any one of said sequences comprising a mutation.

In another preferred embodiment, the nucleic acid molecule encodes a heavy chain variable domain (V_{H}) that utilizes a human V_{H}1 family gene sequence. In various embodiments, the nucleic acid molecule utilizes a human V_{H}1 family gene, a human D gene and a human J_{H} gene.

In some embodiments, the nucleic acid molecule comprises a nucleotide sequence that encodes at least a portion of the V_{H} amino acid sequence of an antibody selected from 11, 71, 73, 85, 15, 95, 84, 10, 69, 79, or 94, a variant thereof, or said sequence having conservative amino acid mutations and/or a total of three or fewer non-conservative amino acid substitutions. In various embodiments, the sequence encodes one or more CDR regions, preferably a CDR3 region, all three CDR regions, or the entire V_{H} domain.

In some embodiments, the nucleic acid molecule comprises a nucleotide sequence that encodes the amino acid sequence of any one of antibodies 11, 71, 73, 85, 15, 95, 84, 10, 69, 79, or 94 (SEQ ID NOS: 12-22, respectively). In some preferred embodiments, the nucleic acid molecule comprises at least a portion of the nucleotide sequence of SEQ ID NOS: 1-11 (encoding antibodies 11, 71, 73, 85, 15, 95, 84, 10, 69, 79, or 94, respectively). In some embodiments, said portion encodes a CDR3 region, all three CDR regions, or a V_{H} domain.

In some embodiments, the nucleic acid molecule encodes a V_{H} amino acid sequence that is at least 70%, 75%, 80%, 85%, 90%, 95%, 97%, 98% or 99% identical to the V_{H} amino acid sequence of any one of antibodies 11, 71, 73, 85, 15, 95, 84, 10, 69, 79, or 94, or to an amino acid sequence depicted in of any one of SEQ ID NOS: 23-33. Nucleic acid molecules of the invention include nucleic acids that hybridize under highly stringent conditions, such as those described above, to a nucleotide sequence encoding an amino acid sequence depicted in any one of SEQ ID NOS: 23-33, or to a nucleotide sequence depicted in any one of SEQ ID NOS: 45-55.

In another embodiment, the nucleic acid encodes a full-length heavy chain or a heavy chain comprising an amino acid sequence selected from the group consisting of SEQ ID NOS: 23-33, or any one of said amino acid sequences comprising a mutation. In some embodiments, the nucleic acid may comprise the nucleotide sequence of any one of SEQ ID NOS: 45-55, or any one of said nucleotide sequence comprising a mutation.

A nucleic acid molecule encoding the heavy or light chain of an anti-WNE antibody or portions thereof can be isolated from any source that produces such antibody. In various embodiments, the nucleic acid molecules are isolated from a B cell obtained from an animal immunized with WNE or from an immortalized cell derived from such a B cell that expresses an anti-WNE antibody. Methods of isolating mRNA encoding an antibody are well-known in the art. See, e.g., Sambrook et al. The mRNA may be used to produce cDNA for use in the polymerase chain reaction (PCR) or cDNA cloning of antibody genes. In some embodiments, the nucleic acid molecule is isolated from a hybridoma that has as one of its fusion partners a human immunoglobulin-producing cell from a non-human transgenic animal. In another embodiment, the human immunoglobulin producing cell is isolated from a mouse transgenic animal. In another embodiment, the human immunoglobulin-producing cell is from a non-human, non-mouse transgenic animal. In another embodiment, the nucleic acid is isolated from a non-human, non-transgenic animal. The nucleic acid molecules isolated from a non-human, non-transgenic animal may be used, e.g., for humanized antibodies.

In some embodiments, a nucleic acid encoding a heavy chain of an anti-WNE antibody of the invention can comprise a nucleotide sequence encoding a V_{H} domain of the invention joined in-frame to a nucleotide sequence encoding a heavy chain constant domain from any source. Similarly, a nucleic acid molecule encoding a light chain of an anti-WNE antibody of the invention can comprise a nucleotide sequence encoding a V_{L} domain of the invention joined in-frame to a nucleotide sequence encoding a light chain constant domain from any source.

In a further aspect of the invention, nucleic acid molecules encoding the variable domain of the heavy (V_{H}) and/or light (V_{L}) chains are “converted” to full-length antibody genes. In one embodiment, nucleic acid molecules encoding the V_{H} or V_{L} domains are converted to full-length antibody genes by insertion into an expression vector already encoding heavy chain constant (C_{H}) or light chain constant (C_{L}) domains, respectively, such that the V_{H} segment is operatively linked to the C_{H} segment(s) within the vector, and/or the V_{L} segment is operatively linked to the C_{L} segment within the vector. In another embodiment, nucleic acid molecules encoding the V_{H} and/or V_{L} domains are converted into full-length antibody genes by linking, e.g., ligating, a nucleic acid molecule encoding a V_{H} and/or V_{L} domains to a nucleic acid molecule encoding a C_{H} and/or C_{L} domain using standard molecular biological techniques. Nucleic acid sequences of human heavy and light chain immunoglobulin constant domain genes are known in the art. See, e.g., Kabat et al., Sequences of Proteins of Immunological Interest, 5th Ed., NIH Publ. No. 91-3242, 1991. Nucleic acid molecules encoding the full-length heavy and/or light chains may then be expressed from a cell into which they have been introduced and the anti-WNE antibody isolated.

The nucleic acid molecules may be used to recombinantly express large quantities of anti-WNE antibodies. The nucleic acid molecules also may be used to produce chimeric antibodies, bispecific antibodies, single chain antibodies, immunoadhesins, diabodies, mutated antibodies and antibody derivatives, as described further below. If the nucleic acid molecules are derived from a non-human, non-transgenic animal, the nucleic acid molecules may be used for antibody humanization, also as described below.

In another embodiment, a nucleic acid molecule of the invention is used as a probe or PCR primer for a specific antibody sequence. For instance, the nucleic acid can be used as a probe in diagnostic methods or as a PCR primer to amplify regions of DNA that could be used, inter alia, to isolate additional nucleic acid molecules encoding variable domains of anti-WNE antibodies. In some embodiments, the nucleic acid molecules are oligonucleotides. In some embodiments, the oligonucleotides are from highly variable domains of the heavy and light chains of the antibody of interest. In some embodiments, the oligonucleotides encode all or a part of one or more of the CDRs of antibodies 11, 71, 73, 85, 15, 95, 84, 10, 69, 79, or 94 or variants thereof.

### Vectors

The invention provides vectors comprising nucleic acid molecules that encode the heavy chain of an anti-WNE antibody of the invention or an antigen-binding portion thereof. The invention also provides vectors comprising nucleic acid molecules that encode the light chain of such antibodies or antigen-binding portion thereof. The invention further provides vectors comprising nucleic acid molecules encoding fusion proteins, modified antibodies, antibody fragments, and probes thereof.

In some embodiments, the anti-WNE antibodies or antigen-binding portions of the invention are expressed by inserting DNAs encoding partial or full-length light and heavy chains, obtained as described above, into expression vectors such that the genes are operatively linked to necessary expression control sequences such as transcriptional and translational control sequences. Expression vectors include plasmids, retroviruses, adenoviruses, adeno-associated viruses (AAV), plant viruses such as cauliflower mosaic virus, tobacco mosaic virus, cosmids, YACs, EBV derived episomes, and the like. The antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors. In a preferred embodiment, both genes are inserted into the same expression vector. The antibody genes are inserted into the expression vector by standard methods (e.g., ligation of complementary restriction sites on the antibody gene fragment and vector, or blunt end ligation if no restriction sites are present).

A convenient vector is one that encodes a functionally complete human C_{H} or C_{L} immunoglobulin sequence, with appropriate restriction sites engineered so that any V_{H} or V_{L} sequence can easily be inserted and expressed, as described above. In such vectors, splicing usually occurs between the splice donor site in the inserted J region and the splice acceptor site preceding the human C domain, and also at the splice regions that occur within the human C_{H} exons. Polyadenylation and transcription termination occur at native chromosomal sites downstream of the coding regions. The recombinant expression vector also can encode a signal peptide that facilitates secretion of the antibody chain from a host cell. The antibody chain gene may be cloned into the vector such that the signal peptide is linked in-frame to the amino terminus of the immunoglobulin chain. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. No. 5,168,062, U.S. Pat. No. 4,510,245 and U.S. Pat. No. 4,968,615. Methods for expressing antibodies in plants, including a description of promoters and vectors, as well as transformation of plants is known in the art. See, e.g., U.S. Pat. No. 6,517,529, incorporated herein by reference. Methods of expressing polypeptides in bacterial cells or fungal cells, e.g., yeast cells, are also well known in the art.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, incorporated herein by reference). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification), the neo gene (for G418 selection), and the glutamate synthetase gene.

Non-Hybridoma Host Cells and Methods of Recombinantly Producing Protein

Nucleic acid molecules encoding anti-WNE antibodies and vectors comprising these nucleic acid molecules can be used for transfection of a suitable mammalian, insect, plant, bacterial or yeast host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors. Methods of transforming cells are well known in the art. See, e.g., U.S. Pat. Nos. 4,399,216, 4,912,040, 4,740,461, and 4,959,455, incorporated herein by reference). Methods of transforming plant cells are well known in the art, including, e.g., Agrobacterium-mediated transformation, biolistic transformation, direct injection, electroporation and viral transformation. Methods of transforming bacterial and yeast cells are also well known in the art.

Mammalian cell lines available as hosts for expression are well known in the art and include many immortalized cell lines available from the American Type Culture Collection (ATCC). These include, inter alia, Chinese hamster ovary (CHO) cells, NSO cells, SP2 cells, HEK-293T cells, NIH-3T3 cells, HeLa cells, baby hamster kidney (BHK) cells, African green monkey kidney cells (COS), human hepatocellular carcinoma cells (e.g., Hep G2), A549 cells, and a number of other cell lines. Cell lines of particular preference are selected through determining which cell lines have high expression levels. Other cell lines that may be used are insect cell lines, such as Sf9 or Sf21 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. Plant host cells include, e.g., Nicotiana, Arabidopsis, duckweed, corn, wheat, potato, etc. Bacterial host cells include E. coli and Streptomyces species. Yeast host cells include Schizosaccharomyces pombe, Saccharomyces cerevisiae and Pichia pastoris.

Further, expression of antibodies of the invention from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, 0 323 997 and 0 338 841.

It is likely that antibodies expressed by different cell lines or in transgenic animals will have different glycosylation from each other. However, all antibodies encoded by the nucleic acid molecules provided herein, or comprising the amino acid sequences provided herein are part of the instant invention, regardless of the glycosylation of the antibodies.

### Transgenic Animals and Plants

Anti-WNE antibodies of the invention also can be produced transgenically through the generation of a mammal or plant that is transgenic for the immunoglobulin heavy and light chain sequences of interest and production of the antibody in a recoverable form therefrom. In connection with the transgenic production in mammals, anti-WNE antibodies can be produced in, and recovered from, the milk of goats, cows, or other mammals. See, e.g., U.S. Pat. Nos. 5,827,690, 5,756,687, 5,750,172, and 5,741,957, incorporated herein by reference. In some embodiments, non-human transgenic animals that comprise human immunoglobulin loci are immunized with WNE or an immunogenic portion thereof, as described above. Methods for making antibodies in plants are described, e.g., in U.S. Pat. Nos. 6,046,037 and 5,959,177, incorporated herein by reference.

In some embodiments, non-human transgenic animals or plants are produced by introducing one or more nucleic acid molecules encoding an anti-WNE antibody of the invention into the animal or plant by standard transgenic techniques. See Hogan and U.S. Pat. No. 6,417,429, supra. The transgenic cells used for making the transgenic animal can be embryonic stem cells or somatic cells or a fertilized egg. The transgenic non-human organisms can be chimeric, nonchimeric heterozygotes, and nonchimeric homozygotes. See, e.g., Hogan et al., Manipulating the Mouse Embryo: A Laboratory Manual 2^{nd} ed., Cold Spring Harbor Press (1999); Jackson et al., Mouse Genetics and Transgenics: A Practical Approach, Oxford University Press (2000); and Pinkert, Transgenic Animal Technology: A Laboratory Handbook, Academic Press (1999), all incorporated herein by reference. In some embodiments, the transgenic non-human animals have a targeted disruption and replacement by a targeting construct that encodes a heavy chain and/or a light chain of interest. In a preferred embodiment, the transgenic animals comprise and express nucleic acid molecules encoding heavy and light chains of an anti-WNE antibody, preferably human WNE. In some embodiments, the transgenic animals comprise nucleic acid molecules encoding a modified antibody such as a single-chain antibody, a chimeric antibody or a humanized antibody. The anti-WNE antibodies may be made in any transgenic animal. In a preferred embodiment, the non-human animals are mice, rats, sheep, pigs, goats, cattle or horses. The non-human transgenic animal expresses said encoded polypeptides in blood, milk, urine, saliva, tears, mucus and other bodily fluids.

### Phage Display Libraries

The invention provides a method for producing an anti-WNE antibody or antigen-binding portion thereof comprising the steps of synthesizing a library of human antibodies, including human scFvs, on phage, screening the library with WNE or a portion thereof, isolating phage that bind WNE, and obtaining the antibody from the phage. By way of example, one method for preparing the library of antibodies for use in phage display techniques comprises the steps of immunizing a non-human animal comprising human immunoglobulin loci with WNE or an antigenic portion thereof to create an immune response, extracting antibody-producing cells from the immunized animal; isolating RNA encoding heavy and light chains of antibodies of the invention from the extracted cells, reverse transcribing the RNA to produce cDNA, amplifying the cDNA using primers, and inserting the cDNA into a phage display vector such that antibodies are expressed on the phage. Recombinant anti-WNE antibodies of the invention may be obtained in this way.

Recombinant anti-WNE human antibodies of the invention can be isolated by screening a recombinant combinatorial antibody library. Preferably the library is a scFv phage display library, generated using human V_{L} and V_{H} cDNAs prepared from mRNA isolated from B cells. Methods for preparing and screening such libraries are known in the art. Kits for generating phage display libraries are commercially available (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, WO 92/09690; Fuchs et al., Bio/Technology 9:1370-1372 (1991); Hay et al., Hum. Antibod. Hybridomas 3:81-85 (1992); Huse et al., Science 246:1275-1281 (1989); McCafferty et al., Nature 348:552-554 (1990); Griffiths et al., EMBO J. 12:725-734 (1993); Hawkins et al., J. Mol. Biol. 226:889-896 (1992); Clackson et al., Nature 352:624-628 (1991); Gram et al., Proc. Natl. Acad. Sci. USA 89:3576-3580 (1992); Garrad et al., BioTechnology 9:1373-1377 (1991); Hoogenboom et al., Nuc. Acid Res. 19:4133-4137 (1991); and Barbas et al., Proc. Natl. Acad. Sci. USA 88:7978-7982 (1991), all incorporated herein by reference.

In one embodiment, to isolate and produce human anti-WNE antibodies with the desired characteristics, a human anti-WNE antibody as described herein is first used to select human heavy and light chain sequences having similar binding activity toward WNE, using the epitope imprinting methods described in PCT Publication No. WO 93/06213, incorporated herein by reference. The antibody libraries used in this method are preferably scFv libraries prepared and screened as described in PCT Publication No. WO 92/01047, McCafferty et al., Nature 348:552-554 (1990); and Griffiths et al., EMBO J. 12:725-734 (1993), all incorporated herein by reference.

Once initial human V_{L} and V_{H} domains are selected, “mix and match” experiments can be performed, in which different pairs of the initially selected V_{L} and V_{H} segments are screened for WNE binding to select preferred V_{L}/V_{H} pair combinations. Additionally, to further improve the quality of the antibody, the V_{L} and V_{H} segments of the preferred V_{L}/V_{H} pair(s) can be randomly mutated, preferably within the CDR3 region of V_{H} and/or V_{L}, in a process analogous to the in vivo somatic mutation process responsible for affinity maturation of antibodies during a natural immune response. This in vitro affinity maturation can be accomplished by amplifying V_{H} and V_{L} domains using PCR primers complimentary to the V_{H} CDR3 or V_{L} CDR3, respectively, which primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated V_{H} and V_{L} segments can be re-screened for binding to WNE.

Following screening and isolation of an anti-WNE antibody of the invention from a recombinant immunoglobulin display library, nucleic acids encoding the selected antibody can be recovered from the display package (e.g., from the phage genome) and subcloned into other expression vectors by standard recombinant DNA techniques. If desired, the nucleic acid can further be manipulated to create other antibody forms of the invention. To express a recombinant human antibody isolated by screening of a combinatorial library, the DNA encoding the antibody is cloned into a recombinant expression vector and introduced into a mammalian host cell, as described above.

### Class Switching

Another aspect of the invention provides a method for converting the class or subclass of an anti-WNE antibody to another class or subclass. In some embodiments, a nucleic acid molecule encoding a V_{L} or V_{H} that does not include sequences encoding C_{L} or C_{H} is isolated using methods well-known in the art. The nucleic acid molecule then is operatively linked to a nucleic acid sequence encoding a C_{L} or C_{H} from a desired immunoglobulin class or subclass. This can be achieved using a vector or nucleic acid molecule that comprises a C_{L} or C_{H} chain, as described above. For example, an anti-WNE antibody that was originally IgM can be class switched to an IgG. Further, the class switching may be used to convert one IgG subclass to another, e.g., from IgG1 to IgG2. Another method for producing an antibody of the invention comprising a desired isotype comprises isolating a nucleic acid encoding a heavy chain of an anti-WNE antibody and a nucleic acid encoding a light chain of an anti-WNE antibody, isolating the sequence encoding the V_{H} domain, ligating the V_{H} sequence to a sequence encoding a heavy chain constant domain of the desired isotype, expressing the light chain gene and the heavy chain construct in a cell, and collecting the anti-WNE antibody with the desired isotype.

### Deimmunized Antibodies

In another aspect of the invention, the antibody may be deimmunized to reduce its immunogenicity using the techniques described in, e.g., PCT Publication Nos. WO98/52976 and WO00/34317 (incorporated herein by reference).

### Mutated Antibodies

In another embodiment, the nucleic acid molecules, vectors and host cells may be used to make mutated anti-WNE antibodies. The antibodies may be mutated in the variable domains of the heavy and/or light chains, e.g., to alter a binding property of the antibody. For example, a mutation may be made in one or more of the CDR regions to increase or decrease the K_{D} of the antibody for WNE, to increase or decrease k_{off}, or to alter the binding specificity of the antibody. Techniques in site-directed mutagenesis are well-known in the art. See, e.g., Sambrook et al. and Ausubel et al., supra. In another embodiment, one or more mutations are made at an amino acid residue that is known to be changed compared to the germline in antibody 11, 71, 73, 85, 15, 95, 84, 10, 69, 79, or 94. The mutations may be made in a CDR region or framework region of a variable domain, or in a constant domain. In a preferred embodiment, the mutations are made in a variable domain. In some embodiments, one or more mutations are made at an amino acid residue that is known to be changed compared to the germline in a CDR region or framework region of a variable domain of an amino acid sequence selected from SEQ ID NOS: 12-22 or whose nucleic acid sequence is presented in SEQ ID NOS: 1-11.

In another embodiment, the framework region is mutated so that the resulting framework region(s) have the amino acid sequence of the corresponding germline gene. A mutation may be made in a framework region or constant domain to increase the half-life of the anti-WNE antibody. See, e.g., PCT Publication No. WO 00/09560, incorporated herein by reference. A mutation in a framework region or constant domain also can be made to alter the immunogenicity of the antibody, to provide a site for covalent or non-covalent binding to another molecule, or to alter such properties as complement fixation, FcR binding and antibody-dependent cell-mediated cytotoxicity (ADCC). According to the invention, a single antibody may have mutations in any one or more of the CDRs or framework regions of the variable domain or in the constant domain.

In some embodiments, there are from 1 to 8, including any number in between, amino acid mutations in either the V_{H} or V_{L} domains of the mutated anti-WNE antibody compared to the anti-WNE antibody prior to mutation. In any of the above, the mutations may occur in one or more CDR regions. Further, any of the mutations can be conservative amino acid substitutions. In some embodiments, there are no more than 5, 4, 3, 2, or 1 amino acid changes in the constant domains.

### Modified Antibodies

In another embodiment, a fusion antibody or immunoadhesin may be made that comprises all or a portion of an anti-WNE antibody of the invention linked to another polypeptide. In a preferred embodiment, only the variable domains of the anti-WNE antibody are linked to the polypeptide. In another preferred embodiment, the V_{H} domain of an anti-WNE antibody is linked to a first polypeptide, while the V_{L} domain of an anti-WNE antibody is linked to a second polypeptide that associates with the first polypeptide in a manner such that the V_{H} and V_{L} domains can interact with one another to form an antigen binding site. In another preferred embodiment, the V_{H} domain is separated from the V_{L} domain by a linker such that the V_{H} and V_{L} domains can interact with one another (see below under Single Chain Antibodies). The V_{H}-linker-V_{L} antibody is then linked to the polypeptide of interest. The polypeptide may be a therapeutic agent, such as a toxin, growth factor or other regulatory protein, or may be a diagnostic agent, such as an enzyme that may be easily visualized, such as horseradish peroxidase. Other polypeptides that may be linked to an antibody described herein include a polyhistidine tag or a maltose binding protein. In addition, fusion antibodies can be created in which two (or more) single-chain antibodies are linked to one another. This is useful if one wants to create a divalent or polyvalent antibody on a single polypeptide chain, or if one wants to create a bispecific antibody.

To create a single chain antibody (scFv), the V_{H}- and V_{L}-encoding DNA fragments are operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} domains joined by the flexible linker. See, e.g., Bird et al., Science 242:423-426 (1988); Huston et al., Proc. Natl. Acad. Sci. USA 85:5879-5883 (1988); McCafferty et al., Nature 348:552-554 (1990). The single chain antibody may be monovalent, if only a single V_{H} and V_{L} are used, bivalent, if two V_{H} and V_{L} are used, or polyvalent, if more than two V_{H} and V_{L} are used. Bispecific or polyvalent antibodies may be generated that bind specifically to WNE and to another molecule. Single chain antibodies may be modified by fusion to an Fc region (Example I). The Fc region can be an IgG1, IgG2, IgG3, or IgG4.

In other embodiments, other modified antibodies may be prepared using anti-WNE antibody encoding nucleic acid molecules. For instance, “Kappa bodies” (111 et al., Protein Eng. 10: 949-57 (1997)), “Minibodies” (Martin et al., EMBO J. 13: 5303-9 (1994)), “Diabodies” (Holliger et al., Proc. Natl. Acad. Sci. USA 90: 6444-6448 (1993)), or “Janusins” (Traunecker et al., EMBO J. 10:3655-3659 (1991) and Traunecker et al., Int. J. Cancer (Suppl.) 7:51-52 (1992)) may be prepared using standard molecular biological techniques following the teachings of the specification.

Bispecific antibodies or antigen-binding fragments can be produced by a variety of methods including fusion of hybridomas or linking of Fab′ fragments. See, e.g., Songsivilai & Lachmann, Clin. Exp. Immunol. 79: 315-321 (1990), Kostelny et al., J. Immunol. 148:1547-1553 (1992). In addition, bispecific antibodies may be formed as “diabodies” or “Janusins.” In some embodiments, the bispecific antibody binds to two different epitopes of WNE. In some embodiments, the bispecific antibody has a first heavy chain and a first light chain from antibody 11, 71, 73, 85, 15, 95, 84, 10, 69, 79, or 94 and an additional antibody heavy chain and light chain.

In some embodiments, the modified antibodies described above are prepared using one or more of the variable domains or CDR regions from a human anti-WNE antibody provided herein.

### Derivatized and Labeled Antibodies

An anti-WNE antibody or antigen-binding portion of the invention can be derivatized or linked to another molecule (e.g., another peptide or protein). In general, the antibodies or portion thereof are derivatized such that the WNE binding is not affected adversely by the derivatization or labeling. Accordingly, the antibodies and antibody portions of the invention are intended to include both intact and modified forms of the human anti-WNE antibodies described herein. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detection agent, a cytotoxic agent, a pharmaceutical agent, and/or a protein or peptide that can mediate association of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Another type of derivatized antibody is a labeled antibody. Useful detection agents with which an antibody or antigen-binding portion of the invention may be derivatized include fluorescent compounds, including fluorescein, fluorescein isothiocyanate, rhodamine, 5-dimethylamine-1-naphthalenesulfonyl chloride, phycoerythrin, lanthanide phosphors and the like. An antibody can also be labeled with enzymes that are useful for detection, such as horseradish peroxidase, β-galactosidase, luciferase, alkaline phosphatase, glucose oxidase and the like. When an antibody is labeled with a detectable enzyme, it is detected by adding additional reagents that the enzyme uses to produce a reaction product that can be discerned. For example, when the agent horseradish peroxidase is present, the addition of hydrogen peroxide and diaminobenzidine leads to a colored reaction product, which is detectable. An antibody can also be labeled with biotin, and detected through indirect measurement of avidin or streptavidin binding. An antibody can also be labeled with a predetermined polypeptide epitope recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

An anti-WNE antibody can also be labeled with a radiolabeled amino acid. The radiolabel can be used for both diagnostic and therapeutic purposes. Examples of labels for polypeptides include, but are not limited to, the following radioisotopes or radionuclides—³H, ¹⁴C, ¹⁵N, ³⁵S, ⁹⁰Y, ⁹⁹Tc, ¹¹¹In, ¹²⁵I, and ¹¹³I.

An anti-WNE antibody can also be derivatized with a chemical group such as polyethylene glycol (PEG), a methyl or ethyl group, or a carbohydrate group. These groups are useful to improve the biological characteristics of the antibody, e.g., to increase serum half-life or to increase tissue binding.

### Pharmaceutical Compositions and Kits

The invention relates to compositions comprising a human anti-WNE antibody for the treatment of patients in need of a therapeutic procedure including, but not limited to, treating, inhibiting, or preventing a West Nile virus infection. In some embodiments, the subject of treatment is a human. In other embodiments, the subject is a veterinary subject. Anti-WNE antibodies of the invention and compositions comprising them can be administered in combination with one or more other therapeutic, diagnostic, or prophylactic agents. In some embodiments, one or more anti-WNE antibodies of the invention can be used as a vaccine or as adjuvants to a vaccine. Treatment may involve administration of one or more anti-WNE antibodies of the invention, or antigen-binding fragments thereof, alone or with a pharmaceutically acceptable carrier.

Anti-WNE antibodies of the invention and compositions comprising them can be administered in combination with one or more other therapeutic, diagnostic or prophylactic agents. Such additional agents may be included in the same composition or administered separately.

As used herein, “pharmaceutically acceptable carrier” means any and all solvents, dispersion media, coatings, antibacterial and antifungal agents, isotonic and absorption delaying agents, and the like that are physiologically compatible. Some examples of pharmaceutically acceptable carriers are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

The compositions of this invention may be in a variety of forms, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans. In some embodiments, the mode of administration is parenteral (e.g., intravenous, subcutaneous, intraperitoneal, intramuscular). In some embodiments, the antibody is administered by intravenous infusion or injection. In another embodiment, the antibody is administered by intramuscular or subcutaneous injection. In some embodiments, the antibody is delivered to the brain of a subject in need thereof in order to bypass the blood-brain barrier in cases of flaviviral encephalitis. In a preferred embodiment, the antibody is administered intrathecally.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the anti-WNE antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies of the present invention can be administered by a variety of methods known in the art. As will be appreciated by the skilled artisan, the route and/or mode of administration will vary depending upon the desired results.

In certain embodiments, the antibody compositions may be prepared with a carrier that will protect the antibody against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems (J. R. Robinson, ed., Marcel Dekker, Inc., New York, 1978).

In certain embodiments, an anti-WNE antibody of the invention can be orally administered, for example, with an inert diluent or an assimilable edible carrier. The compound (and other ingredients, if desired) can also be enclosed in a hard or soft shell gelatin capsule, compressed into tablets, or incorporated directly into the subject's diet. For oral therapeutic administration, the anti-WNE antibodies can be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Additional active compounds also can be incorporated into the compositions. In certain embodiments, an anti-WNE antibody of the invention is co-formulated with and/or co-administered with one or more additional therapeutic agents. These agents include, without limitation, antibodies that bind other targets, antiviral agents, or peptide analogues that inhibit WNE.

Protective anti-WNE antibodies of the invention and compositions comprising them also may be administered in combination with other therapeutic regimens such as, for example, in combination with purine or pyrimidine analogs (e.g., ribavirin), interferons (e.g., interferon alpha), or human immunoglobulins.

The compositions of the invention may include a “therapeutically effective amount” or a “prophylactically effective amount” of an antibody or antigen-binding portion of the invention. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

Dosage regimens can be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the anti-WNE antibody or portion thereof and the particular therapeutic or prophylactic effect to be achieved, and (b) the limitations inherent in the art of compounding such an antibody for the treatment of sensitivity in individuals.

An exemplary, non-limiting range for a therapeutically or prophylactically effective amount of an antibody or antibody portion of the invention is 0.025 to 50 mg/kg, more preferably 0.1 to 50 mg/kg, more preferably 0.1-25, 0.1 to 10 or 0.1 to 3 mg/kg. In some embodiments, a formulation contains 5 mg/ml of antibody in a buffer of 20 mM sodium citrate, pH 5.5, 140 mM NaCl, and 0.2 mg/ml polysorbate 80. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Another aspect of the present invention provides kits comprising an anti-WNE antibody or antibody portion of the invention or a composition comprising such an antibody. A kit may include, in addition to the antibody or composition, diagnostic, prophylactic or therapeutic agents. A kit can also include instructions for use in a diagnostic, prophylactic or therapeutic method. In a preferred embodiment, the kit includes an antibody or a composition comprising it and a diagnostic agent that can be used in a method described below. In another preferred embodiment, the kit includes the antibody or a composition comprising it and one or more therapeutic agents that can be used in a method described below.

### Diagnostic Methods of Use

In another aspect, the invention provides diagnostic methods. The anti-WNE antibodies of the invention can be used to detect WNE in a biological sample in vitro or in vivo. In one embodiment, the invention provides a method for diagnosing a West Nile virus infection in a subject in need thereof, comprising contacting a biological sample from the subject with an antibody of the invention, determining the presence of a West Nile virus infection in the subject by detecting bound antibody, comparing the amount of bound antibody in the biological sample of the subject with that of a normal reference subject or standard, and diagnosing the presence or absence of a West Nile virus infection in the subject.

The anti-WNE antibodies can be used in a conventional immunoassay, including, without limitation, an ELISA, an RIA, flow cytometry, tissue immunohistochemistry, Western blot or immunoprecipitation. The anti-WNE antibodies of the invention can be used to detect WNE from different West Nile virus isolates, such as for example, West Nile virus isolate 2741 and West Nile virus isolate 2000. In another embodiment, the anti-WNE antibodies can be used to detect E protein of other flaviviruses, such as for example, SLEV and dengue viruses. In certain embodiments, an antibody of the present invention can be used to detect different strains of dengue virus, such as DENV-2 and DENV-4, which may be present in a biological sample.

The invention provides a method for detecting a WNE in a biological sample comprising contacting the biological sample with an anti-WNE antibody of the invention and detecting the bound antibody. In one embodiment, the anti-WNE antibody is directly labeled with a detectable label. In another embodiment, the anti-WNE antibody (the first antibody) is unlabeled and a second antibody or other molecule that can bind the anti-WNE antibody is labeled. As is well known to one of skill in the art, a second antibody is chosen that is able to specifically bind the particular species and class of the first antibody. For example, if the anti-WNE antibody is a human IgG, then the secondary antibody could be an anti-human-IgG. Other molecules that can bind to antibodies include, without limitation, Protein A and Protein G, both of which are available commercially, e.g., from Pierce Chemical Co.

Suitable labels for the antibody or secondary antibody have been disclosed supra, and include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹²⁵I, ¹³¹I, ³⁵S or ³H.

In other embodiments, WNE can be assayed in a biological sample by a competition immunoassay utilizing WNE standards labeled with a detectable substance and an unlabeled anti-WNE antibody. In this assay, the biological sample, the labeled WNE standards and the anti-WNE antibody are combined and the amount of labeled WNE standard bound to the unlabeled antibody is determined. The amount of WNE in the biological sample is inversely proportional to the amount of labeled WNE standard bound to the anti-WNE antibody.

### Prophylactic and Therapeutic Methods of Use

The antibodies of the present invention also can be used in vivo, for example as prophylactics or therapeutics. One advantage of using the human anti-WNE antibodies of the present invention as therapeutics in human patients is that they may safely be used in vivo without eliciting a substantial immune response to the antibody upon administration, unlike antibodies of non-human origin or with humanized or chimeric antibodies.

In another embodiment, the invention provides a method for preventing, inhibiting, or treating infection by a Dengue virus or a flavivirus of the Japanese Encephalitis Antigenic Complex (JEAC) by administering a protective anti-WNE antibody to a patient in need thereof. Viruses in the Japanese Encephalitis Antigenic Complex include at least West Nile Virus, St. Louis Encephalitis Virus, Murray Valley Encephalitis Virus, Japanese Encephalitis Virus, and Kunjin Virus. The Japanese Encephalitis Antigenic Complex is sometimes considered also to include Alfuy, Cacipacore, Koutango, Rocio, Stratford, Usutu, and Yaounde viruses. In preferred embodiments, the antibody that is administered therapeutically is selected from antibodies 11, 71, 73, 85, 15, 95, 84, 10, 69, 79, or 94, variants thereof or an antibody that comprises the heavy chain CDRs or variable domain, the light chain CDRs or variable domain, or an antigen-binding portion thereof. In one embodiment, the anti-WNE antibody is a human, chimeric or humanized antibody. In a preferred embodiment, the anti-WNE antibody is a human antibody, and the patient is a human patient. In other embodiments, the antibody can be administered to a non-human mammal for veterinary purposes or as an animal model of human disease. Such animal models may be useful for evaluating the therapeutic efficacy of antibodies of this invention.

The antibody may be administered once, but more preferably is administered multiple times. The antibody may be administered from three times daily to once every six months or longer. The administering may be on a schedule such as three times daily, twice daily, once daily, once every two days, once every three days, once weekly, once every two weeks, once every month, once every two months, once every three months and once every six months. The antibody may also be administered continuously via a minipump. The antibody may be administered via an intrathecal, oral, mucosal, buccal, intranasal, inhalable, intravenous, subcutaneous, intramuscular, parenteral, or topical route. The antibody may be administered once, at least twice or for at least the period of time until the condition is treated, palliated or cured. The antibody will generally be administered as part of a pharmaceutical composition as described supra. The dosage of antibody may be in the range of 0.1-100 mg/kg, more preferably 0.5-50 mg/kg, and more preferably 1-20 mg/kg. The serum concentration of the antibody may be measured by any method known in the art.

In another aspect, the anti-WNE antibody may be co-administered with other therapeutic agents. In some embodiments, the anti-WNE antibody combination therapy is administered along with other antiviral agents including purine or pyrimidine analogs, interferon alpha, human immunoglobulin, steroids, anti-convulsants, or osmotic agents (e.g., mannitol). In yet another preferred embodiment, the antibody will be administered with another antibody. For example, the anti-WNE antibody may be administered with an antibody or other agent that is known to inhibit a West Nile virus or other flaviviral infection, such as a protective anti-Dengue E protein antibody.

Co-administration of the antibody with an additional therapeutic agent (combination therapy) encompasses administering a pharmaceutical composition comprising the anti-WNE antibody and the additional therapeutic agent as well as administering two or more separate pharmaceutical compositions, one comprising the anti-WNE antibody and the other(s) comprising the additional therapeutic agent(s). Further, although co-administration or combination therapy generally means that the antibody and additional therapeutic agents are administered at the same time as one another, it also encompasses instances in which the antibody and additional therapeutic agents are administered at different times. For instance, the antibody may be administered once every three days, while the additional therapeutic agent is administered once daily. Alternatively, the antibody may be administered prior to or subsequent to treatment of the disorder with the additional therapeutic agent, for example after a patient has failed therapy with the additional agent. Similarly, administration of the anti-WNE antibody may be administered prior to or subsequent to other therapy, such as supportive antiviral therapy (e.g., ribavirin, interferon alpha) or other immunotherapy.

The antibody and one or more additional therapeutic agents (the combination therapy) may be administered once, twice or at least the period of time until the condition is treated, palliated or cured. Preferably, the combination therapy is administered multiple times. The combination therapy may be administered from three times daily to once every six months. The administering may be on a schedule such as three times daily, twice daily, once daily, once every two days, once every three days, once weekly, once every two weeks, once every month, once every two months, once every three months and once every six months, or may be administered continuously via a minipump. The combination therapy may be administered via an oral, mucosal, buccal, intranasal, inhalable, intravenous, subcutaneous, intramuscular, parenteral, or topical route.

In a still further embodiment, the anti-WNE antibody is labeled with a radiolabel, an immunotoxin or a toxin, or is a fusion protein comprising a toxic peptide. The anti-WNE antibody or anti-WNE antibody fusion protein directs the radiolabel, immunotoxin, toxin or toxic peptide to the WNE-expressing virus or cell.

In another aspect, the anti-WNE antibody may be used to treat non-flaviviral diseases or conditions that are associated with West Nile virus infection. In one embodiment, the anti-WNE antibody slows the progress of the non-flaviviral pathological state.

### Gene Therapy

The nucleic acid molecules of the present invention can be administered to a patient in need thereof via gene therapy. The therapy may be either in vivo or ex vivo. In a preferred embodiment, nucleic acid molecules encoding both a heavy chain and a light chain are administered to a patient. In a more preferred embodiment, the nucleic acid molecules are administered such that they are stably integrated into chromosomes of B cells because these cells are specialized for producing antibodies. In a preferred embodiment, precursor B cells are transfected or infected ex vivo and re-transplanted into a patient in need thereof. In another embodiment, precursor B cells or other cells are infected in vivo using a virus known to infect the cell type of interest. Typical vectors used for gene therapy include liposomes, plasmids and viral vectors. Exemplary viral vectors are retroviruses, adenoviruses and adeno-associated viruses. After infection either in vivo or ex vivo, levels of antibody expression can be monitored by taking a sample from the treated patient and using any immunoassay known in the art or discussed herein.

In a preferred embodiment, the gene therapy method comprises administering an isolated nucleic acid molecule encoding the heavy chain or an antigen-binding portion thereof of an anti-WNE antibody and expressing the nucleic acid molecule. In another embodiment, the gene therapy method comprises administering an isolated nucleic acid molecule encoding the light chain or an antigen-binding portion thereof of an anti-WNE antibody and expressing the nucleic acid molecule. In a more preferred method, the gene therapy method comprises administering an isolated nucleic acid molecule encoding the heavy chain or an antigen-binding portion thereof and an isolated nucleic acid molecule encoding the light chain or the antigen-binding portion thereof of an anti-WNE antibody of the invention and expressing the nucleic acid molecules. The gene therapy method may also comprise the administering another anti-viral agent.

### Anti-WNE Peptides

In a further aspect, the invention provides West Nile virus E protein peptides. In particular, the invention provides peptide 29 (amino acids 281-300 of the WNE protein) recognized by exemplified protective anti-WNE antibodies. This region, part of DI the contact between the DI and DIII interface involved in membrane fusion and contains a glycosaminoglycan (GAG)-binding motif. The invention further provides peptide 39 (amino acids 381-400), also recognized by exemplified protective anti-WNE antibodies.

In another aspect, the invention provides a method for producing or eliciting a protective anti-WNE antibody, including an antibody that cross-protects against Dengue virus and/or flaviviruses in the Japanese Encephalitis Antigenic Complex, by immunizing a subject with peptide 29 and/or peptide 39. Alternatively, one can screen a phage display antibody (including scFv) library, such as a human or primate library, with peptide 29 and/or peptide 39 to identify additional protective anti-WNE antibodies.

In order that this invention may be better understood, the following examples are set forth. These examples are for purposes of illustration only and are not to be construed as limiting the scope of the invention in any manner.

### EXAMPLE I

### Selection of Phage Display Antibody Library

Single-chain variable fragments (scFvs) against the WNV E protein were identified using a phage display screen. Two human, nonimmune phage display libraries were screened; both were created from the B cells of normal, presumed non-WNV immune humans and contain between 12 and 15 billion unique phage displayed in the phagemid vector pFarber as fusions with phage coat protein III (Sui, J., Li, W. et al. (2004) Proc Natl Acad Sci USA 101:2536-2541; Ledizet, M., Kar, K., Foellmer, H. G., Wang, T., Bushmich S. L., Anderson, J. F., Fikrig, E, and Koski, R. A. (2005) A recombinant protein vaccine against West Nile virus. Vaccine in press). Recombinant WNV-E protein ectodomain (rWNV-E) that was expressed in Drosophila S2 cells and highly purified (Wong, S. J. et al. (2004) J Clin Microbiol 42:65-72; Ledizet, M., Kar, K., Foellmer, H. G., Wang, T., Bushmich S. L., Anderson, J. F., Fikrig, E, and Koski, R. A. 2005. A recombinant protein vaccine against West Nile virus. Vaccine in press) was coated overnight on Maxisorp immunotubes (Nalge Nunc International) at a concentration of 15 μg/ml in phosphate buffered saline (PBS), pH 7.4. Phage (5×1012 pfu) were added to the tubes and allowed to bind for two hours at room temperature. Nonspecifically absorbed phages were removed by extensive washing (15 times with PBS/0.05% Tween-20, 15 times with PBS), and bound phage were eluted in 100 mM triethylamine. Eluted phage were allowed to infect Escherichia coli TG1 cells, and pooled phage were rescued by VCS M13 helper phage, and concentrated by polyethylene glycol/NaCl precipitation (Sui, J. et al. (2004) Proc Natl Acad Sci USA 101:2536-2541]. Four rounds of selection were performed. Following the second, third, and fourth rounds of selection, individual TG1 colonies were screened by ELISA.

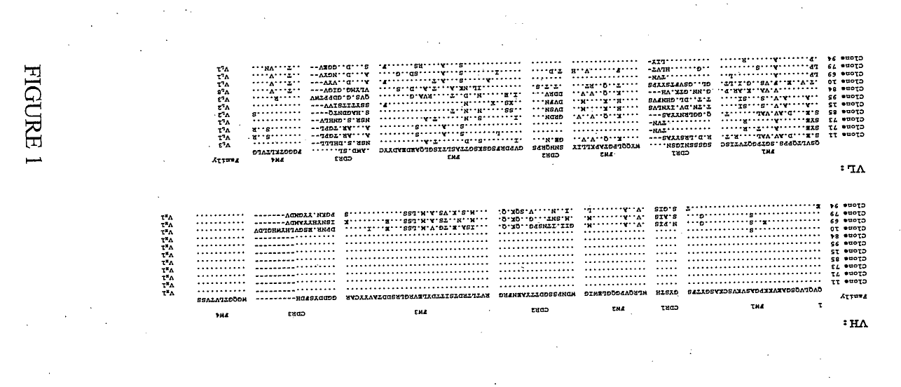

For ELISA screening, 96-well microtiter plates were coated overnight with rWNV-E (10 μg/ml) in PBS, pH 7.4. Plates were blocked for 1 hour with PBS-2% milk. After extensive washing with PBS-Tween 20, plates were incubated with anti-M13-HRP (Amersham) to detect the M13 tag on the scFvs, and developed with Sure Blue Microwell Peroxidase substrate (Kirkegaard & Perry Laboratories, Inc (KPL), Gaithersburg, Md.), stopped after 10 minutes with TMB Stop Solution (KPL), and the OD₄₅₀ was measured. Phage that bound to rWNV-E with an A450 value >1.0 were scored as positive. Phage clones that bound to rWNV-E were sequenced and their corresponding amino acid sequences aligned (see FIG. 1).

Eleven unique anti-rWNV-E scFvs were then identified by DNA sequence analysis. Amino acid sequences predicted by sequence analysis of the VH and VL of the eleven scFv genes are shown in FIG. 1. All of the VH sequences were in the VH1 human gene family; all of the scFvs had lambda light chains and utilized the VL1, VL2, VL3, and VL8 human gene families. ScFvs 10, 11, 15, 71, 73, 84, 85, and 95 had identical or nearly identical VH sequences, while scFvs 69, 79, and 94 had distinct VH sequences, particularly in CDR2 and CDR3, the primary domain involved in antigen binding. VL sequences were distinct for all of the eleven scFvs.

Expression and Purification of scFvs and scFv-Fc Fusions

Antibody genes of rWNV-E specific scFvs were excised from the phagemid vector by Not I-NcoI digestion and ligated into the prokaryotic expression vector, pSyn (Bai, J. et al. (2003) J Biol Chem 278:1433-1442), which adds C-terminal c-myc and His-6 tags. E. coli XL-1 Blue cells were transformed with the plasmid and individual colonies were screened by restriction digestion, and the insert DNA sequences were verified. For scFv expression, bacteria were grown in 2×YT medium containing 0.1% glucose and 100 μg/ml ampicillin, and were induced overnight with 1 mM isopropyl-B-D-thiogalactopyranoside at 30° C. Bacterial cultures were pelleted and resuspended in PBS containing Complete Protease Inhibitor Cocktail (Roche), and the cultures were sonicated for 2 minutes. The homogenate was centrifuged to remove insoluble debris, and the protein was precipitated from the supernatant with 4.1 M ammonium sulfate. The precipitated protein purified on a Ni2+ immobilized chelating sepharose column (Amersham). Purified scFvs were dialyzed overnight against PBS, concentrated, and stored at −70° C.

Purified scFvs were tested for their binding activity against rWNV-E by ELISA. 96-well microtiter plates were coated overnight with rWNV-E (1 μg/ml in PBS). Plates were blocked with PBS-2% milk, followed by incubation with serial 10-fold dilutions of the scFvs for 1 hour at room temperature. Monoclonal anti-His6 antibody conjugated to horse radish peroxidase (HRP) (1:4000; Invitrogen Corporation, Carlsbad, Calif.) was added for 1 hour and the plates developed and read as described above.

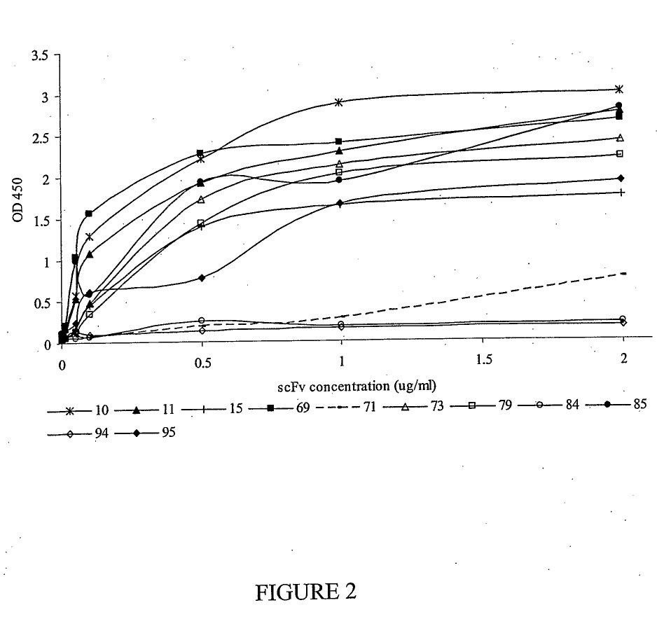

The eleven scFvs tagged with c-myc and His-6 epitopes were expressed in E. coli and purified by immobilized metal affinity chromatography. The binding activity of the scFvs for rWNV-E was examined by both ELISA and Western blot. In the ELISA assay, 8 of the 11 scFvs bound with high affinity to rWNV-E, while scFv 71 displayed an intermediate level of binding, and scFvs 84 and 94 did not bind well to rWNV-E (FIG. 2).

For production of scFv-Fc fusions, antibody genes were excised from the phagemid vector by NotI-SfiI digestion and cloned into the vector pcDNA 3.1 Hinge which contains the Fc fragment of human IgG1. ScFv-Fc fusions were expressed in 293T cells by transient calcium phosphate transfection and purified by protein A Sepharose (Amersham) affinity chromatography. ScFv-Fc fusions were screened for binding activity against rWNV-E by ELISA as described above using anti-human IgG-HRP (1:10000; Sigma) as a secondary antibody.

### Serum and Rabbit IgG Preparation

A New Zealand white rabbit was immunized with 50 μg of rWNV-E in complete Freund's adjuvant, boosted twice at three week intervals with the same antigen in incomplete Freund's adjuvant, and the serum was collected. The IgG fraction was purified from the rabbit antiserum by Protein G affinity chromatography (Amersham). Nonimmune rabbit serum was obtained from animals with no history of flavivirus exposure and lacked reactivity to the E protein as measured by ELISA and Western blot. Normal, non-flavivirus immune human IgG1 was obtained from Sigma.

The F(ab′)2 fraction was prepared from the purified anti-rWNV-E IgG fraction and from 79 scFv-Fc by digestion with immobilized pepsin (Immunopure F(ab′)2 Preparation Kit, Pierce). Intact IgG and Fc fragments were removed from the digests by Protein A column chromatography, and the F(ab′)2 fraction was further purified by Sephacryl S-100 column chromatography in PBS. Protein concentration was determined by BCA protein assays (Pierce).

### EXAMPLE II

Selected scFvs were converted to scFv-Fc fusions. The Fc expression vector used in these experiments, pcDNA 3.1 Hinge, contains the hinge, CH2, and CH3 domains of human IgG1, but lacks CH1.

Seven scFv-Fcs were assessed for neutralization of WNV in vitro using a standard Vero cell plaque assay (described below). WNV strain 2741 was used in the studies described herein, with the exception of the murine ADE experiment, where WNV 2000 was used. SLEV, strain Parton P-3, and DENV-2, New Guinea C were used in their respective assays.

All of the seven scFv-Fcs tested neutralized WNV plaque formation by greater than 80%, at minimum concentrations ranging from 1.25 to 12.5 ug/ml (Table 2). Consistent with its lower affinity for rWNV-E, 84 scFv-Fc had a higher PRNT80. Addition of the Fc region increased viral binding. All seven scFvs also neutralized WNV plaque formation in the assay but were 10-20 fold less effective.

TABLE 2
Plaque-reduction neutralization titers (PRNT) against WNV.

| scFv-Fc | PRNT₈₀ titer (μg/ml) |
|---|---|
| 11 | 1.25 |
| 15 | 1.25 |
| 71 | 2.5 |
| 73 | 1.25 |
| 79 | 5 |
| 84 | 12.5 |
| 95 | 1.25 |

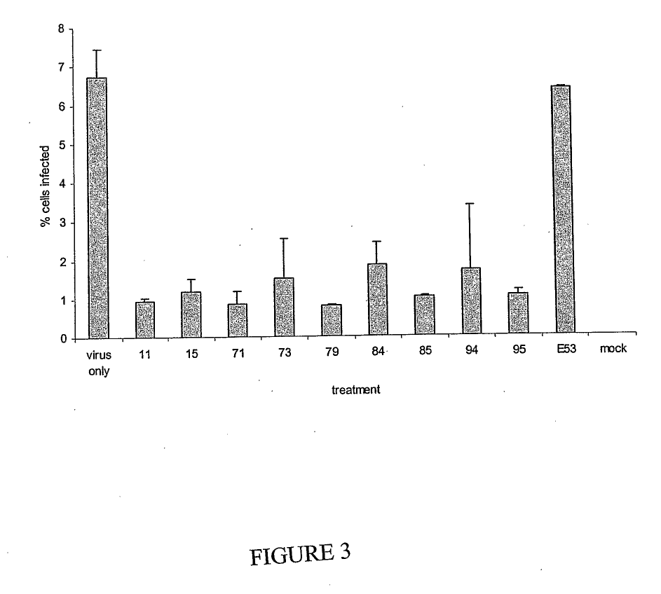

The scFv-Fcs were next assessed for their ability to neutralize other flaviviruses in vitro. Nine of the scFv-Fcs were tested in a neutralization assay with Dengue 2 (DENV-2), and one of the scFv-Fcs, 79 scFv-Fc, was tested for neutralization of St. Louis Encephalitis virus (SLEV) and Vesicular Stomatitis virus containing the E gene of hepatitis C virus (VSV-HCV). All of the scFv-Fcs tested gave greater than 75% neutralization of DENV-2 at a concentration of 12.5 μg/ml (FIG. 3). Only 79 scFv-Fc was tested against DENV-2 at lower concentrations, and it reduced plaque formation by greater than 80% at a concentration of 5 μg/ml. DENV-2 was not neutralized by the control antibodies E53 or anti-OspB. However, it is expected that a number other scFv-Fcs will neutralize DENV-2 at lower concentrations. 79 scFv-Fc also effectively neutralized SLEV, with a PRNT80 of 5 μg/ml. Because SLEV is more closely related to WNV than DENV-2, the scFv-Fvs that have not yet been tested would be expected to neutralize SLEV. 79 scFv-Fc did not neutralize VSV, an unrelated virus, expressing the HCV envelope glycoprotein.

### Virus Neutralization Assays

Vero cells were seeded in 6-well plates at a density of 3×10⁵ cells/ml 24 hours before infection. Serial dilutions of IgG, scFvs, or scFv-Fcs were mixed with 100 plaque forming units (PFU) virus at and 100 ml incubated for 1 hour at 37° C./5% CO₂. The virus-antibody mixture was added to the cell monolayers and incubated for another hour. Cells were overlaid with 3-4 ml of 1% agarose in cell culture medium, and after four days a second overlay of 2.5 ml 1% agarose/medium containing 0.01% neutral red was added to visualize plaques. The plaque reduction neutralization assay for DENV-2 was conducted as above but cells were incubated for 6 days before the second overlay. The PRNT80 value was calculated as the minimum concentration of antibody giving an 80% reduction in plaques.

Alternatively, in some experiments, plaques were visualized with crystal violet staining. Briefly, cells were overlaid as above with 1% agarose/DMEM/5% FCS. Instead of a second overlay, cells were fixed in 10% formaldehyde for 1 hour, the agarose overlay removed, and the plaques stained with 1% crystal violet/10% ethanol.

DENV-2 neutralization was also assayed in a FACS based infectivity assay. Briefly, Vero cells were plated overnight in 6-well plates at a density of 3×10⁵ cells/ml. Virus (MOI=0.1) and serial dilutions of antibodies were mixed and incubated for an hour at 37° C./5% CO₂, and then added to the cell monolayers for an additional hour of incubation. Media was then added to the cells, and the cells were incubated for 24 hours. Cells were then detached from the 6-well plates by treatment with 1× trypsin-EDTA, washed twice in PBS-10% FCS, and fixed and permeabilized with Cytofix-Cytoperm (BD Biosciences). Staining for DENV-2 was performed by incubation of the cells for 30 minutes on ice with an anti-Dengue mAB (Chemicon, clone D3-2H2-21-9, Temecula, Calif.), diluted 1:200, followed by incubation with the secondary antibody anti-mouse-PE, diluted 1:50 (Sigma). Cells were counted on a FACScan and data analyzed using Cell Quest software.

Vesicular stomatitis virus (VSV) containing the E gene of hepatitis C virus (HCV) and a green fluorescent protein (GFP) was used in the HCV neutralization assay [Buonocore, L. et al (2002) J Virol 76:6865-6872]. Virus was incubated with antibodies at 37° C. for 30 minutes and then added to Huh-7 cells for an additional 3 hours of incubation. Cells were overlaid with DMEM/5% FCS containing 1% methylcellulose and the number of GFP+ plaques counted after 48-72 hours.

### EXAMPLE III

### Affinity Measurements by Biacore

The binding kinetics and affinity of the scFvs for rWNV-E were measured by surface plasmon resonance (Biacore 3000, Uppsala, Sweden). ScFvs (30-50 μg/ml) were covalently immobilized to a NTA Sensor Chip (Biacore) via their histidine tag. The running buffer used contained 0.01 M HEPES (pH 7.4) with 0.15 M NaCl₂, 50 μM EDTA and 0.005% Surfactant P20 (Biacore). The NTA surface was activated with 500 μM NiCl2 in running buffer. All experiments were run at a flow rate of 20 μl/minute in HBS-EP buffer (Biacore). The chip surface was regenerated with 0.01 M HEPES with 0.15 M NaCl, 0.35 M EDTA and 0.005% Surfactant P20, pH 8.3. The binding kinetic parameters were measured by varying the molar concentration (0.704 to 440 nM) of rWNV-E and analyzed using BIA-EVALUATION software (Biacore). Results are shown in Table 3 below.

To measure the binding affinity of the scFv-Fcs to rWNV-E, scFv-Fcs (30 μg/ml) were first immobilized using goat anti-human IgG (30 μg/ml in 10 mM sodium acetate, pH 5.0; Bethyl Laboratories, Montgomery, Tex.) that was covalently coupled to a CM4 Sensor Chip (Biacore) using an amine coupling kit (Biacore). Assays with the scFv-Fcs were run at a flow rate of 20 μl/minute in HBS-EP buffer (Biacore), and the chip surface was regenerated with 10 mM glycine, pH 1.8. The binding kinetics were measured and analyzed as above. (See Table 3).

TABLE 3
Kinetic rates and binding affinity of scFvs
and selected scFv-Fcs for rWNV-E.

| Antibody | Kₒₙ, M⁻¹s⁻¹ | K_{off}, s⁻¹ | Kₐ, M⁻¹ | K_{d}, M |
|---|---|---|---|---|
| 10 scFv | 5.61 × 10⁵ | .0212 | 2.64 × 10⁷ | 3.78 × 10⁻⁸ |
| 11 scFv | 3.26 × 10⁵ | 1.61 × 10⁻³ | 2.03 × 10⁸ | 4.92 × 10⁻⁹ |
| 15 scFv | 1.33 × 10⁵ | 1.28 × 10⁻³ | 1.04 × 10⁸ | 9.65 × 10⁻⁹ |
| 69 scFv | 2.13 × 10⁵ | 1.26 × 10⁻³ | 1.69 × 10⁷ | 5.92 × 10⁻⁸ |
| 71 scFv | 9.39 × 10⁴ | 2.58 × 10⁻³ | 3.64 × 10⁷ | 2.75 × 10⁻⁸ |
| 73 scFv | 5.17 × 10⁵ | 2.25 × 10⁻³ | 2.30 × 10⁸ | 4.35 × 10⁻⁹ |
| 79 scFv | 1.25 × 10⁵ | 6.7 × 10⁻⁴ | 1.87 × 10⁸ | 5.35 × 10⁻⁹ |
| 79 scFv-Fc | 2.62 × 10⁴ | 3.58 × 10⁻⁷ | 7.34 × 10¹⁰ | 1.36 × 10⁻¹¹ |
| 84 scFv | 1.81 × 10⁵ | 6.85 × 10⁻³ | 2.65 × 10⁷ | 3.78 × 10⁻⁸ |
| 85 scFv | 5.01 × 10⁶ | 6.73 × 10⁻³ | 7.45 × 10⁸ | 1.34 × 10⁻⁹ |
| 95 scFv | 2.35 × 10⁵ | 7.15 × 10⁻⁴ | 3.29 × 10⁸ | 3.04 × 10⁻⁹ |
| 95 scFv-Fc | 3.20 × 10⁵ | 1.09 × 10⁻⁵ | 2.94 × 10¹⁰ | 3.40 × 10⁻¹¹ |

### EXAMPLE IV

### In Vivo Protection by scFv and scFv-Fcs

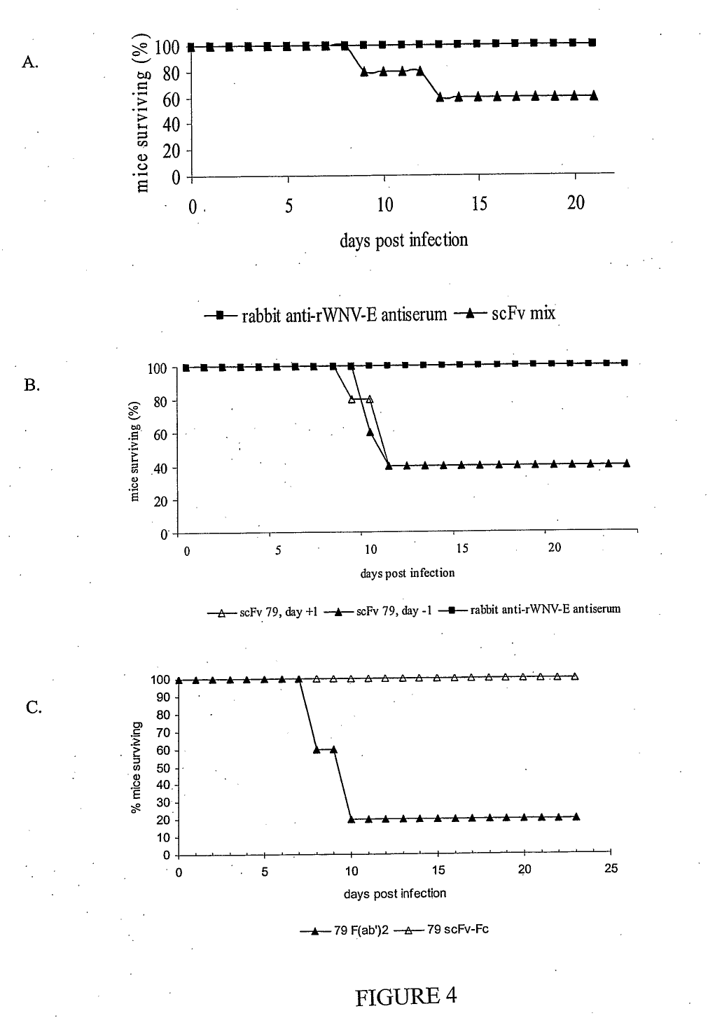

The ability of the scFvs and the scFv-Fc fusion proteins to protect mice from a lethal dose of WNV was assessed. A mixture of the scFvs (all scFvs except clone 10) provided partial protection against lethal WNV infection (FIG. 4A). Additionally, administration of 100 μg of a single representative scFv, 79, either 1 day before or 1 day after infection provided partial protection against viral challenge (FIG. 4B). To confirm the critical role of the Fc region in protection, and to show that the bivalency of the scFv-Fc antibodies is not sufficient for protection, mice were immunized with F(ab′)₂ fragments derived from 79 scFv-Fc. 79 F(ab′)₂ was not protective in mice (FIG. 4C), which is consistent with our previous studies showing that rabbit F(ab′)₂ fragments were only partially protective.

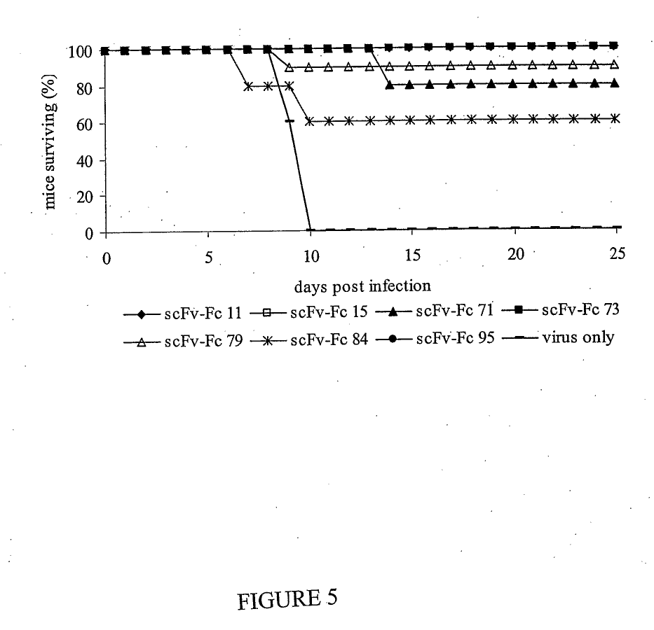

The scFv-Fcs were both prophylactically and therapeutically active. Administration of 100 μg of any of the scFv-Fcs 1 day prior to infection with a lethal dose of WNV significantly increased survival (FIG. 5). ScFv-Fcs 11, 15, 73, 85 and 95 protected 100% of mice challenged, scFv-Fc 79 provided 90% protection, scFv-Fc 71 and 94 provided 80% protection, and scFv-Fc 84 provided 60% protection.

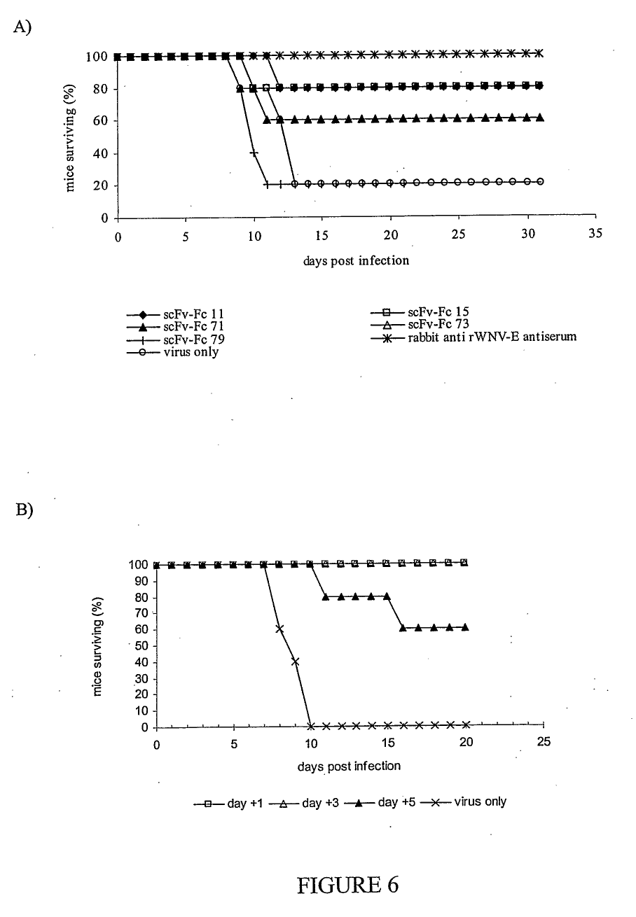

We next examined the therapeutic activity of several of the scFv-Fcs. Both scFv-Fc 11 and 15 were therapeutically active, with 80% of infected mice surviving when given two injections consisting of 100 μg of antibody at days 1 and 4 after infection (FIG. 6A). Further experiments showed that scFv-Fc 11 was more effective therapeutically than was scFv-Fc 15, with complete protection against a lethal challenge dose up to 3 days after infection by scFv-Fc 11 (FIG. 6B). A dose of 250 μg of scFv-Fc 11 protected 100% mice when given 1 day after infection, and a dose of 500 μg protected 100% of mice at day 3. Mice given 500 μg of scFv-Fc 11 at day 5 were partially protected.

### Mouse Passive Immunization and Viral Challenge

Groups of 5 to 10 female C57BL/6 mice (Jackson Laboratories) between 4 and 6 weeks of age were used in all experiments. Mice were injected with 102-103 PFU WNV intraperitoneally (i.p.). In experiments with rabbit antibodies, mice were inoculated i.p. with the indicated doses of serum at times ranging from 1 day prior to 5 days post WNV infection. Human IgG₁, scFvs and scFv-Fcs were administered subcutaneously either 24 hours before or after virus inoculation. Survival was recorded daily until no further deaths had occurred for at least 7 days. Mice were weighed at the same time daily to the nearest 1/10th of a gram. All animal experiments were conducted in accordance with the Yale University Animal Care and Use Committee regulations.

### EXAMPLE V

### Serum Levels of scFv-Fcs

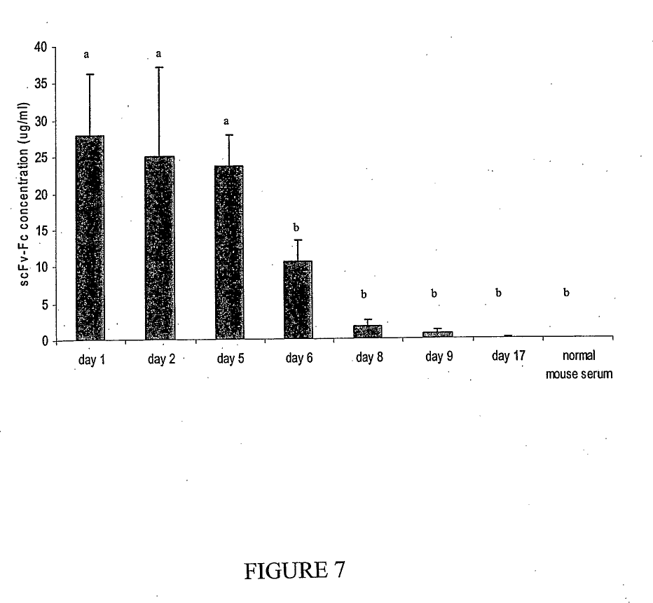

In order to determine the potential window of therapeutic efficacy, the residence time of a representative antibody, 79 scFv-Fc, was detected daily in serum samples of mice. The concentration of scFv-Fc was measured using a human IgG capture ELISA. As shown in FIG. 7, 79 scFv-Fc is present in high levels in mouse serum for 5 days following administration. The level of scFv-Fc drops significantly between days 5 and 6 (p<0.01, ANOVA followed by Tukey's post hoc test). The letters a and b refer to statistically different groups (p<0.01) by ANOVA followed by Tukey's post hoc test to compare means.

Thus, administration of antibody prior to infection allows for a significant quantity remaining in the blood before the viral load peaks in the serum at day four and disseminates into peripheral sites of infection. Because of potential immune responses directed at a heterospecific human antibody, a human scFv-Fc or IgG would be expected to be present for significantly longer in a human than in a mouse model, thus increasing the duration of antibody efficacy.

### Human IgG Capture ELISA

The amount of scFv-Fc present in mouse serum was quantified using the Human IgG ELISA Quantitation Kit (Bethyl Laboratories, Montgomery, Tex.). Goat anti-human IgG antibody (1 μg/well) was coated on ELISA plates overnight at 4° C. in 0.05 Carbonate-Bicarbonate buffer, pH 9.6. After blocking for 30 minutes with 2% BSA-PBS, plates were incubated for 1 hour at room temperature with mouse serum diluted 1:150. After extensive washing, the plates were incubated with goat anti-human-IgG-HRP (1:15,000), developed with True Blue Microwell Peroxidase (KPL) and the reaction stopped after 10 minutes with TMB Stop Solution (KPL). The OD₄₅₀ was measured, and the amount of scFv-Fc present in the mouse serum was calculated by comparing to the amount of IgG in standard human reference serum.

### EXAMPLE VI

### Antibody Dependent Enhancement of Infection

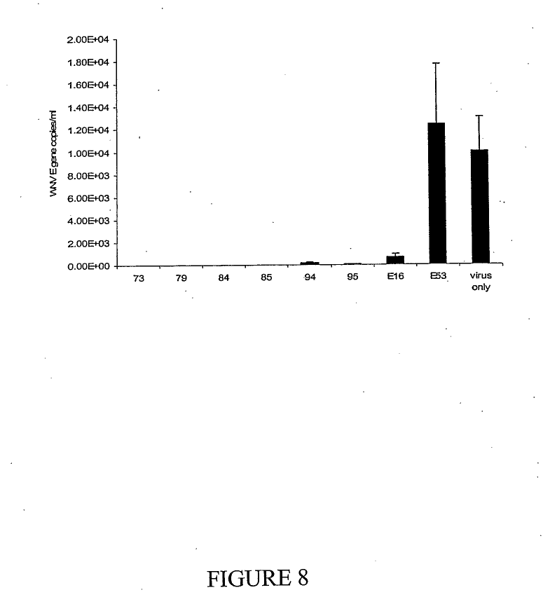

To determine whether the antibodies produced antibody dependent enhancement (ADE) in vitro, experiments were done with both murine and human macrophages. ScFv-Fcs were preincubated with 100 pfu WNV before incubation with macrophages for 4 hours. After extensive washing, the cells were cultivated for 24 hours, and the supernatants harvested for determination of viral load by QPCR (FIG. 8). Little enhancement was observed with any of the scFv-Fcs from this study using mouse cell line J774. Significant enhancement of infection was seen, however, with the control antibody E53. To investigate the possibility that the human Fc region of the scFv-Fcs would not be able to activate mouse Fc receptors, we conducted similar experiments using in human macrophages. In cultivated human macrophages, no enhancement of infection was observed for any of the scFv-Fcs in this study as assessed by viral replication. Again, enhancement was seen in human macrophages with E53, albeit at a much lower level in comparison to the level observed in murine cells, suggesting that enhancement occurs with greater efficiency through conspecific Fc receptors.

### Antibody Enhancement of Infection Assay

Human monocyte-derived macrophages were obtained as previously described [Montgomery, R. R. et al. (2002) J Infect Dis 185:1773-1779]. Briefly, monocytes were isolated from heparinized blood from healthy volunteers using Ficoll-hypaque (Pharmacia, Piscataway, N.J.) and plated in 0.1 ml RPMI/20% heat inactivated human serum at a density of 3×10⁶ cells per well in a 12-well plate. Non-adherent cells were rinsed away gently with warm RPMI after 1-2 hours of incubation at 37° C./5% CO₂, and the cells were cultured for an additional 6-8 days to obtain macrophages.

Antibody-virus complexes were prepared by incubation of 100 PFU WNV with 200 μl of antibodies (25 μg/ml in DMEM) for 45 minutes at 4° C. The virus-antibody mixture was then added to the cells and incubated for a further 4 hours at 37° C. Following incubation, the cells were washed 5 times with DMEM/1% FBS, 1 ml DMEM/10% FBS was added to the cells, and they were incubated for 24 hours at 37° C. The supernatants were harvested and the amount of virus titrated by plaque assay on Vero cells or RNA was extracted and quantified by QPCR to measure 24-hour viral replication and release.

### Quantitative PCR (QPCR)

RNA was extracted from blood and tissues of infected mice using the RNeasy Kit (Qiagen, Valencia, Calif.). Complementary DNA (cDNA) was synthesized from RNA using the ProSTAR First-strand RT-PCR kit (Stratagene). QPCR was performed on an iCycler (Bio-Rad), using the following amplification cycle: 95° C. for 3 minutes followed by 60 cycles of 95° C. for 30 seconds and 60° C. for 1 minute. Samples were normalized to β-actin levels and the ratio of the amount of amplified E gene to the amount of β-actin was calculated to obtain the relative levels in each sample.

The sequences of the probe and primer sets for the WNV E gene have been described previously [Lanciotti, R. S., and Kerst, A. J. (2001) J Clin Microbiol 39:4506-4513]. The sequences for the mouse B-actin primers (forward, reverse) and probe were as follows: AGAGGGAAATCGTGCTGAC, CAATAGTGATGACCTGGCCG, and CACTGCCGCATCCTCTTCCTCCC. The probes were 5′ labeled with the reporter FAM, and 3′ labeled with the quencher TAMRA. All probes were synthesized by Applied Biosystems.

### EXAMPLE VII

### E Protein Cloning and Mutagenesis

Various E-protein fragments (ectodomain, DIII, and DI/DII) were cloned into a yeast display vector, pYD1 (Invitrogen). This expression vector displays proteins of interest as a fusion with the AGA2 gene of Saccharomyces cerevisiae.

Libraries of DI/DII and DIII mutants were created by error prone PCR. DI/DII and DIII cDNA was amplified by PCR in the presence of 50 mM MgCl₂ and 5 mM MnCl₂ to create random mutations. The PCR product was Bam/Xho digested, and ligated into the pYD1 vector, and transformed into E. coli DH5α. A minimum of 10 clones were selected and sequenced for each library, and the mutation rate calculated. On average, each clone contained 1 mutation. The mutated library was then transformed into S. cerevisiae strain EBY 100. The transformation was grown on minimal dextrose plates containing leucine. Single colonies were grown overnight in YNB-CAA medium containing 2% glucose and display of the fusion protein was induced by the addition of 2% galactose at log-phase. The expression of the fusion protein was monitored for 12-48 hours post induction to determine the optimal induction time for maximum display. Protein display was confirmed by staining with anti-Xpress antibody (Invitrogen).

### E Protein Domain Binding Assay

Yeast cells expressing pYD1, the WNV ectodomain, WNV DIII, or WNV DI/DII were plated in 96 wells plates and incubated for 30 minutes on ice with scFv-Fcs (1 μg/ml) conjugated to Alexa Fluor 647 (Invitrogen/Molecular Probes) at a 1:500 dilution. ScFv-Fc conjugates were prepared according to the manufacturer's directions. Cells were washed 3 times with PBS-1% BSA and the cells were fixed in 1% paraformaldehyde and counted on a FACsCalibur (Becton Dickinson). Data were analyzed with Cell Quest software. Alternatively, unconjugated antibodies were incubated with yeast at a concentration of 50 μg/ml for 30 minutes on ice, followed by incubation with goat anti-human IgG-Alexa Fluor 647 (Invitrogen/Molecular Probes) at a concentration of 1:500 in 1 mg/ml BSA in PBS for an additional 30 minutes. Cells were washed and fixed as above.

### ScFv-Fcs Bind to DI/DII

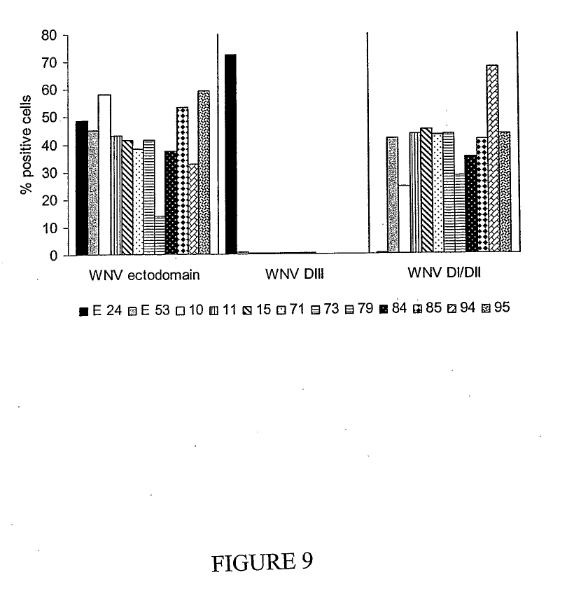

Using the yeast display described above, the WNV E protein epitopes that are recognized by scFv-Fc antibodies described herein was assessed. All of the scFvs studied mapped to either DI/DII or DIII of the WNV E protein. The control mAbs E16 and E53 mapped to DIII and DI/DII, respectively, as shown previously. All of the scFvs in this study mapped to the WNV E ectodomain. More specifically, all bound to DI/DII, and none to DIII (FIG. 9). To further delineate the binding domains, mutated libraries of DIII and DI/DII were created in which key residues were eliminated and/or altered, and binding of the scFv-Fcs to the mutated residues assessed. The mutation rates for the two yeast display libraries were 1% and 0.5% for DI/DII and DIII, respectively. Using the mutated DIII library, E16 was further found to map to S306 and K307 (see Oliphant, T et al. Nat Med (2005) 1 (5):522-530).

### EXAMPLE VIII

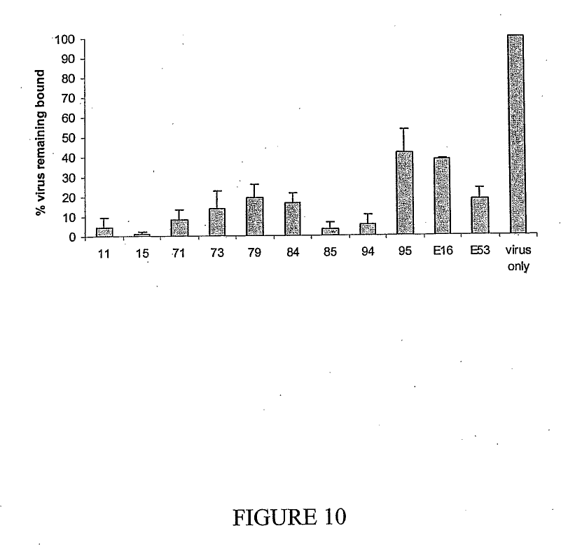

To elucidate the mechanism of scFv-Fc protection, studies were conducted to analyze the ability of scFvs to block virus attachment to Vero cells. As shown in FIG. 10, all of the scFv-Fcs reduced viral binding by at least 50% and the majority were even more highly effective blockers of virus binding to Vero cells.

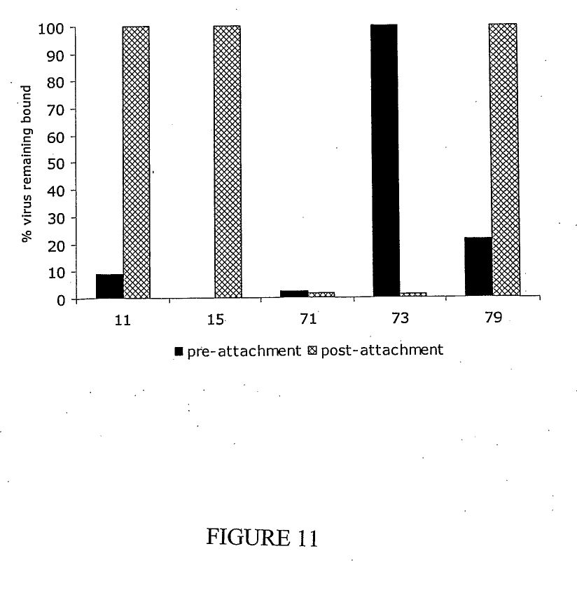

The scFv-Fcs were next assessed for their ability to block virus attachment either pre- or post-adsorption of virus to cells. The pre-adsorption assay (see below) measures the ability of the scFv-Fcs to block virus attachment early in the infection cycle, including by direct adsorption of virus [Crill, W. D., and Roehrig, J. T. (2001) J Virol 75:7769-7773]. In the post-adsorption assay (see below), scFv-Fc is added only after virus has absorbed to cells, and thus reflects the ability of antibodies to block only after viral attachment has occurred. As shown in FIG. 11, scFv-Fcs 11, 15, 71, and 79 all were highly effective at blocking adsorption prior to attachment. In contrast, only scFv-Fcs 71 and 73 blocked attachment post-adsorption. The ability of scFv-Fc 71 to interfere with binding both before and after cell attachments suggests that it recognizes epitopes that remain exposed but are nevertheless important for cell attachment. The finding that scFv-Fc 73 blocked attachment post-adsorption suggests that it recognizes an epitope that is not exposed until virus has bound to the cell surface. Both scFv-Fc 71 and 73 and related compositions could be used as very potent viral entry inhibitors that can target virus at several stages in the attachment process.

### Vero Cell Binding Assays

Vero cells were plated overnight in 12 well plates at a density of 3×10⁵ cells/well. Antibodies were diluted to 50 μg/ml in DMEM/10% FCS and incubated with 1 pfu/μl WNV for 1 hour at 37° C. The antibody-virus mixture was added to the Vero cells and incubated for 2 hours at 4° C. The cells were then washed four times with cold DMEM/10% FCS and once with cold PBS, and lysed directly in the plate in Buffer RLT (Qiagen). RNA was extracted and quantitative PCR performed.

Assays to assess the mechanism of antibody inhibition of attachment were performed essentially as previously described [Crill, W. D., and Roehrig, J. T. (2001) J Virol 75:7769-7773; Hung, S. L. et al. (1999) Virology 257:156-167]. For both assays, Vero cells were plated in 6-well plates at a concentration of 3×10⁵ cells/well overnight. For the preadsorption assay, ten-fold serial dilutions of antibodies were mixed with 100 PFU WNV for 1.5 hours on ice. The antibody-virus complexes were added to the cells and incubated for a further 1.5 hours on ice. For the post-adsorption assay, the cells were first incubated with 100 PFU WNV for 1.5 hours on ice, followed by an additional 1.5 hours of incubation with the antibodies. For both assays, the cells were then washed 3 times with cold PBS, and 1 ml of media was added to the cells and they were incubated for a further 1.5 hours at 37° C. The cells were then treated with 1 ml of 20 mM glycine, pH 3.5 for 1 minute, and washed four times with cold media and once with cold PBS, and then overlaid for a plaque assay the cells were harvested for RNA extraction and QPCR.

### EXAMPLE IX

### WNV Peptides and Peptide ELISA

Thirty-two overlapping 20-mer peptides (see Table 4) spanning the length of the E protein ectodomain were synthesized (Sigma Genosys, The Woodlands, Tex.). Peptides were coated on ELISA plates (Nunc) at a concentration of 10 μg/ml in 0.1 M sodium bicarbonate buffer, pH 9.6, overnight at 4° C. Plates were blocked with 2% BSA-PBS for 1 hour at room temperature, and then incubated with serum or antibodies (1 ug/ml) for an additional 1 hour at room temperature. Depending on the assay, serum was diluted from 1:100-1:500 in 2% BSA-PBS. After extensive washing, plates were incubated with anti-horse-HRP, anti-rabbit-HRP, or anti-human-BRP antibody (1:10,000), for 1 hour at room temperature and developed with Sure Blue Microwell Peroxidase substrate (KPL). The reaction was stopped with TMB Stop Solution (KPL), and the OD₄₅₀ measured.

TABLE 4

Sequences of the 40 20-mer peptides spanning
the length of the E protein ectodomain.

| | Amino | |
|---|---|---|
| Peptide | acid # | Domain |
| 1. FNCLGMSNRDFLEGVSGATW | 1-20 | I |
| 2. FLEGVSGATWVDLVLEGDSC | 11-30 | I |
| 3. VDLVLEGDSCVTIMSKDKPT | 21-40 | I |
| 4. VTIMSKDKPTIDVKMMNMEA | 31-50 | I |
| 5. IDVKMMNMEAANLAEVRSYC | 41-60 | I/II |
| 6. ANLAEVRSYCYLATVSDLST | 51-70 | II |
| 7. YLATVSDLSTKAACPTMGEA | 61-80 | II |
| 8. KAACPTMGEAHNDKRADPAF | 71-90 | II |
| 9. HNDKRADPAFVCRQGVVDRG | 81-100 | II |
| 10. VCRQGVVDRGWGNGCGLFGK | 91-110 | II |
| 11. WGNGCGLFGKGSIDTCAKFA | 101-120 | II |
| 12. GSIDTCAKFACSTKAIGRTI | 111-130 | II |
| 13. CSTKAIGRTILKENIKYEVA | 121-140 | II |
| 14. LKENIKYEVAIFVHGPTTVE | 131-150 | II/I |
| 15. IFVHGPTTVESHGNYSTQVG | 141-160 | I |
| 16. SHGNYSTQVGATQAGRFSIT | 151-170 | I |
| 17. ATQAGRFSITPAAPSYTLKL | 161-180 | I |
| 18. PAAPSYTLKLGEYGEVTVDC | 171-190 | I/II |
| 19. GEYGEVTVDCEPRSGIDTNA | 181-200 | I/II |
| 20. EPRSGIDTNAYYVMTVGTKT | 191-210 | II |
| 21. YYVMTVGTKTFLVHREWFMD | 201-220 | II |
| 22. FLVHREWFMDLNLPWSSAGS | 211-230 | II |
| 23. LNLPWSSAGSTVWRNRETLM | 221-240 | II |
| 24. TVWRNRETLMEFEEPHATKQ | 231-250 | II |
| 25. EFEEPHATKQSVIALGSQEG | 241-260 | II |
| 26. SVIALGSQEGALHQALAGAI | 251-270 | II |
| 27. ALHQALAGAIPVEFSSNTVK | 261-280 | II |
| 28. PVEFSSNTVKLTSGHLKCRV | 271-290 | II/I |
| 29. LTSGHLKCRVKMEKLQLKGT | 281-300 | II/I |
| 30. KMEKLQLKGTTYGVCSKAFK | 291-310 | I |
| 31. TYGVCSKAFKFLGTPADTGH | 301-320 | III |
| 32. FLGTPADTGHGTVVLELQYT | 311-330 | III |
| 33. GTVVLELQYTGTDGPCKVPI | 321-340 | III |
| 34. GTDGPCKVPISSVASLNDLT | 331-350 | III |
| 35. SSVASLNDLTPVGRLVTVNP | 341-360 | III |
| 36. PVGRLVTVNPFVSMATANAK | 351-270 | III |
| 37. FVSMATANAKVLIELEPPFG | 361-380 | III |
| 38. VLIELEPPFGDSYIVVGRGE | 371-390 | III |
| 39. DSYIVVGRGEQQINHHWHKS | 381-400 | III |
| 40. QQINHHWHKSGSSIGKAFTT | 391-410 | III |

### Binding to Linear Epitopes

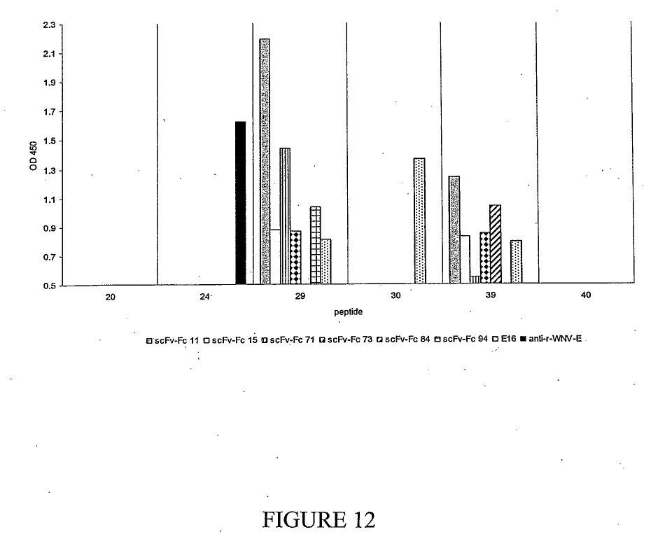

Several of the scFv-Fcs bound strongly to selected peptides in ELISA (FIG. 12). ScFv-Fcs 79, 85, and 95 did not bind to any of the peptides. ScFv-Fcs 11, 15, 71, 73, and 94 all bound strongly only to peptide 29, which encompasses amino acids 281-300 (see FIG. 12). scFv-Fcs, including scFv-Fc 84 also bound to peptide 39, which spans amino acids 381-400. E16, which was previously mapped to S306 and K307, bound to peptide 30, which spans amino acids 291-310. These results are surprising as conformational epitopes have been shown to be important, and this work suggests the particular relevance of amino acids 281-300.

In order to determine whether the scFv-Fcs recognized unique domains of the E protein, the panel of peptides was also used to map binding of sera from horses and rabbits that had been immunized with rWNV-E. As shown in Table 5, immunized rabbits responded strongly to peptides spanning amino acids 231-270, and horse serum reacted primarily with peptides encompassing amino acids 131-170. Serial bleeds from a human infected with WNV were also tested against the peptide panel, with maximal binding found for peptide 10, which spans the flavivirus fusion loop, and 14, which lies at the DI/DII interface. In general, little reactivity was found with any of the DIII region peptides.

TABLE 5
Pattern of binding to WNV E peptides by immune sera.

| | | | | Horse | | Human | |
|---|---|---|---|---|---|---|---|
| | | Rabbit | Im- | Pre- | Im- | Pre- | Im- |
| Domain | Peptide | NRS^{a} | mune | immune | mune | immune | mune |
| I | 1 | | +++^{b} | | | | |
| II | 10 | | | + | ++ | | ++ |
| II | 11 | | | | | | + |
| II/I | 14 | | | + | ++ | | ++ |
| I | 15 | | + | + | ++ | | |
| I | 16 | | + | | | | |
| II | 20 | | | | | | + |
| II | 23 | | | | + | | |
| II | 24 | | +++ | | + | | |
| II | 26 | | +++ | | ++ | | + |
| III | 33 | | ++ | | | | + |
| rWNV-E | | | +++ | | +++ | + | +++ |

^{a}Normal Rabbit Serum (NRS)
^{b}+ OD in ELISA at least 2-fold greater than background
++ OD at least 3-fold greater than background
+++ OD at least 4-fold greater than background

All publications and patent applications cited in this specification are incorporated herein by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### SEQUENCES

Key:
CDRs 1, 2, 3 (protein sequences): underlined
Variable domains (protein sequences): UPPER CASE

SEQ ID NO: 1

ScFv 11 nucleotide sequence
atggcccaggtgcagctggtgcagtctggggctgaggtgaagaagcctgg
ggcctcagtgaaagtctcctgcaaggcttctggatacaccttcagcggct
actctacacactggctgcgacaggtccctggacagggacttgagtggatt
ggatgggacaaccctagtagtggtgacacgacctatgcagagaattttcg
gggcagggtcaccctgaccagggacacgtccatcaccacagattacttgg
aagtgaggggtctaagatctgacgacacggccgtctattattgtgccaga
ggcggagatgactacagctttgaccattggggtcagggcaccctggtcac
cgtctcctcaggtggcggcggttccggaggtggtggttctggcggtggtg
gcagctcttctgagctgactcaggacccagctgtgtctgtggccttggga
cagacagtcaggatcacatgccgaggagacagcctcagaagttattatgc
aagctggtaccaacagaagccaggacaggcccctgtacttgtcatctatg
gtgaaaacaaccgaccctcagggatcccagaccgattctctggctccagc
tcaggagacacagcttccttgaccatcactggggctcaggcggaagatga
ggctgactattactgtaactcccgggacagcagtgatcaccttctcctat
tcggtggagggaccaagttgaccgtcctaggtcagcccaaggctaccccc
tcggcggccgcagaacaaaaactcatctcagaggaagatctgtag

SEQ ID NO: 2

ScFv 71 nucleotide sequence
atggcccaggtgcagctggtgcagtctggggctgaggtgaagaagcctgg
ggcctcagtgaaagtctcctgcaaggcttctggatacaccttcagcggct
actctacacactggctgcgacaggtccctggacagggacttgagtggatt
ggatgggacaaccctagtagtggtgacacgacctatgcagagaattttcg
gggcagggtcaccctgaccagggacacgtccatcaccacagattacttgg
aagtgaggggtctaagatctgacgacacggccgtctattattgtgccaga
ggcggagatgactacagctttgaccattggggtcagggaaccctggtcac
cgtctcctcaggtggcggcggttccggaggtggtggttctggcggtggtg
gcagctcctatgagctgactcagccaccatcagcgtctgggaccaccggg
cagagggtcaccatctcttgttctggaagcagctccaacatcggaagtaa
tactgtaaactggtaccagcagctcccaggaacggcccccaaactcctca
tctatagtaataatcagcggccctcaggggtccctgaccgattatctggc
tccaagtctggcacctcagcctccctggccatcagtggactccagtctga
agatgaggccgattattactgtgctgcgtgggatgcccgcctgactggtc
ccctcttcggcggggggaccaagctaagcgtcctacgtcagcccaaggcc
gccccctcggcggccgcagaacaaaaactcatctcagaggaagatctgt
ag

SEQ ID NO: 3

ScFv 73 nucleotide sequence
atggcccaggtgcagctggtgcagtctggggctgaggtgaagaagcctgg
ggcctcagtgaaagtctcctgcaaggcttctggatacaccttcagcggct
actctacacactggctgcgacaggtccctggacagggacttgagtggatt
ggatgggacaaccctagtagtggtgacacgacctatgcagagaattttcg
gggcagggtcaccctgaccagggacacgtccatcaccacagattacttgg
aagtgaggggtctaagatctgacgacacggccgtctattattgtgccaga
ggcggagatgactacagctttgaccattggggtcagggaccctggtcacc
gtctcctcaggtggcggcggttccggaggtggtggttctggcggtggtgg
cagctcctatgagctgactcagccaccctcagcgtctgggacccccgggc
agagggtcaccatctcttgttctggaagcagctccaacatcggaagtaat
actgtaaactggtaccagcagctcccaggaacggcccccaaactcctcat
ctatagtaataatcagcggccctcaggggtccctgaccgattctctggct
ccaagtctggcacctcagcctccctggccatcagtggactccagtctgaa
gatgaggccgattattactgtgctgcgtgggatgcccgcctgactggtcc
cctcttcggcggggggaccaagctaagcgtcctacgtcagcccaaggccg
ccccctcggcggccgcagaacaaaaactcatctcagaggaagatctgtag

SEQ ID NO: 4

ScFv 85 nucleotide sequence
atggcccaggtgcagctggtgcagtctggggctgaggtgaagaagcctgg
ggcctcagtgaaagtctcctgcaaggcttctggatacaccttcagcggct
actctacacactggctgcgacaggtccctggacagggacttgagtggatt
ggatgggacaaccctagtagtggtgacacgacctatgcagagaattttcg
gggcagggtcaccctgaccagggacacgtccatcaccacagattacttgg
aagtgaggggtctaagatctgacgacacggccgtctattattgtgccaga
ggcggagatgactacagctttgaccattggggtcagggaaccctggtcac
cgtctcctcaggtggcggcggttccggagtggtggttctggcggtggtgg
cagctcttctgagctgactcaggaccctgctgtgtctgtggccttggggc
agacagtcacgatcacatgtcaaggaggcggcctcagaaattattatgca
agttggtaccaacagaagccgggacaggcccctgtccttctcgtctatgg
aagagacaaccggccctcagggatcccagaccgattctctggctccagct
caggaaacacagcttccttgaccatcactggggctcaggcggaagatgag
gctgactattactgtaactcccgggacagcagtggtaaccatctggtgtt
cggcggagggaccaagctgaccgtcctaggtcagcccaaggccaccccct
cggcggccgcagaacaaaaactcatctcagaggaagatctgtag

SEQ ID NO: 5

ScFv 15 nucleotide sequence
atggcccaggtgcagctggtgcagtctggggctgaggtgaagaagcctgg
ggcctcagtgaaagtctcctgcaaggcttctggatacaccttcagcggct
actctacacactggctgcgacaggtccctggacagggacttgagtggatt
ggatgggacaaccctagtagtggtgacacgacctatgcagagaattttcg
gggcagggtcaccctgaccagggacacgtccatcaccacagattacttgg
aagtgaggggtctaagatctgacgacacggccgtctattattgtgccaga
ggcggagatgactacagctttgaccattggggtcagggaaccctggtcac
cgtctcctcaggtggcggcgttccggaggtggtggttctggcggtggtgg
cagccagtctgccctgactcagcctgcctccgtgtctgggtctcctggac
agtcgatcaccatctcctgcactggaaccaacagtgatgttggaatttat
aaccttgtctcctggtaccaacagcacccaggcaaagcccccaaactcat
gatttatgatgtcagtaatcggccctcaggggtttctagtcgcttctctg
gctccaactctgggaacacggccaccctgaccatctctgggctccaggct
gaagatgaggctgattattattgcagcgcacatgcaggcgacaacaccca
attcggcggagggaccaagctgaccgtcctaagtcagcccaaggctgccc
cctcggcggccgcagaacaaaaactcatctcagaggaagatctgtag

SEQ ID NO: 6

ScFv 95 nucleotide sequence
ggaacagcttgaccatgattacgccaagcttgcatgcaaattctatttca
aggagacagtcataatgaaatacctattgcctacggcagccgctggattg
ttattactcgcggcccagccggccatggcccaggtgcagctggtgcagtc
tggggctgaggtgaagaagcctggggcctcagtgaaagtctcctgcaagg
cttctggatacaccttcagcggctactctacacactggctgcgacaggtc
cctggacagggacttgagtggattggatgggacaaccctagtagtggtga
cacgacctatgcagagaatttcggggcagggtcaccctgaccagggacac
gtccatcaccacagattacttggaagtgaggggtctaagatctgacgaca
cggccgtctattattgtgccagaggcggagatgactacagctttgaccat
tggggtcagggaaccctggtcaccgtctcctcaggtggcggcggttccgg
aggtggtggttctggcggtggtggcagccagtctgccctgactcagcctg
cctccgtgtctgggtctcctggacagtcgatcaccatctcctgcactgga
accagcagtgaccttggtggtcacaactttgtctcctggtaccaacagca
cccaggcaaagcccccaaactcatgatttatgatgtctttaatcggccct
caggggtttntagtcgnttctntggctccaagtctggcaacacggcctcc
ctgaccatctctgggctccaggctgaggacgaggctgattatttctgcag
ctcatatacaatcaccagcatcgtggtcttcggcggagggaccaagctga
ccgtcctaggtcagcccaaggccaccccctcggcggccgcagaacaaaaa
ctcatctcagaggaagatctgtag

SEQ ID NO: 7

ScFv 84 nucleotide sequence
atggcccaggtgcagctggtgcagtctggggctgaggtgaagaagcctgg
ggcctcagtgaaagtctcctgcaaggcttctggatacaccttcagcggct
actctacacactggctgcgacaggtccctggacagggacttgagtggatt
ggatgggacaaccctagtagtggtgacacgacctatgcagagaattttcg
gggcagggtcaccctgaccagggacacgtccatcaccacagattacttgg
aagtgaggggtctaagatctgacgacacggccgtctattattgtgccaga
ggcggagatgactacagctttgaccattggggtcagggaaccctggtcac
cgtctcctcaggtggcggcggttccggaggtggtggttctggcggtggtg
gcagccagtctgtgctgactcagccaccctcagtgtcagtggccccagga
aagacggccaggattccctgtgggggaaacaacagtggaactaaaagtgt
gcactggtaccagcagaagccaggccaggcccctgtgctggtcatctatg
atgatagagtccggccctcagggatccctgagcgattctctggctccaac
tctggggacacggccaccctgaccatcagcagggtcgcagccggggatga
ggccgactattactgtcaggtgtcggatggtagtggtgatcctcccactt
gggtgttcggcggagggaccaggctgaccgtcctaggtcagcccaaggct
gccccctcggcggccgcagaacaaaaactcatctcagaggaagatctgt
ag

SEQ ID NO: 8

ScFv 10 nucleotide sequence
atggcccaggtgcagctggtgcagtctggggctgaggtgaagaagcctgg
gtcgtcggtgaaggtctcctgcaaggcttctggatacaccttcagcggct
actctacacactggctgcgacaggtccctggacagggacttgagtggatt
ggatgggacaaccctagtagtggtgacacgacctatgcagagaattttcg
gggcagggtcaccctgaccagggacacgtccatcaccacagattacttgg
aagtgaggggtctaagatctgacgacacggccgtctattattgtgccaga
ggcggagatgactacagctttgaccattggggcagggcaccctggtcacc
gtctcctcaagtggcggcggttccggaggtggtggttctggcggtggtgg
cagccagactgtggtgactcaggagccatcgttctcagtgtcccctggag
ggaccatcacactcacttgtggcttgagctctggctcagtctttactagt
tactaccccagctggtaccagcagaccccaggccaggctccacgcacgct
catctacagcacaaacactcgctcttctggggtccctgatcgcttctctg
gctccatccttgggaacaaagctgccctcaccatcacgggggcccaggca
gatgatgaatctgattattactgtgtcctgtatatgggtagtggcattgg
ggtcttcggaactgggaccaaggtcaccgtcctaggtcagcccaaggctg
ccccctcggcggccgcagaacaaaaactcatctcagaggaagatctgtag

SEQ ID NO: 9

ScFv 69 nucleotide sequence
atggcccaggtgcagctggtgcagtctggggctgaggtgaaggagcctgg
atcttcagtgaaagtctcctgtaaggcttctggaggcaccttcagcaatt
atcctatcagttgggtgcgacaggcccctggacaagggcttgagtggatg
ggagggatcatccccatcactaattcgccaggctatgcacaaaagttcca
gggcagagttacaatttccgcggacgaatcgacgggcacagtctacatgg
agctgagcagcctgagatctgaggacacggccatatattctgtgcaaaag
atccaaatcgctatgagagtgggtactccactattggcacggtttggacg
tctggggccaagggaccacggtcaccgtctcctcaggtggcggcggttcc
ggaggtggtggttctggcggtggtggcagcctgcctgtgctgactcagcc
accctcagcgtcggggacccccgggcagacggttaccctctcttgttctg
gaagcagctccaacatcggaagtaatactgtaaactggtaccagcagctc
ccaggaacggcccccaaactcctcatctatagtaataatcagcggccctc
aggggtccctgaccgattctctgcctccaagtctggcacctcagcctccc
tggccatcactgggctccaggctgaggatgaggctgattatttctgtgca
gcatgggatgacagcctggtttatgtcttcggaactgggaccaaggtcac
cgtcctaggtcagcccaaggctgccccctcggcggccgcagaacaaaaac
tcatctcagaggaagatctgtag

SEQ ID NO: 10

ScFv 79 nucleotide sequence
atggcccaggtgcagctggtgcagtctggggctgagggaagaagcctggg
tcctcggtgaaggtctcctgcaaggcttctggaggcaccttcagcagcta
tgctatcagctgggtgcgacaggcccctggacaagggcttgagtggatgg
gatggatgaattctaacactggtgacacaggctatgcacagaagttccag
ggcagagtcaccatgaccaggaacacctccacaagcacagcctatatgga
gctgagcagcctgagatccgaggacacggccgtctattactgtgcgaaaa
tctccaactaccactattacgctatggacgtctggggccaaggaaccctg
tcaccgtctcctcaggtggcggcggtccggaggtggtggttctggcggtg
gtggcagcctgcctgtgctgactcagccaccctcagcgtctgggacctcc
gggcagacggtcaccatctcctgttctggagggagctccaacatcggaag
tcatcttgtaacctggtaccagcagtttccagggacggccccaaagtcct
catacatactaatgatcagcgaccctctggggtccctgaccgaatctctg
gctccaagtctggcacctcagcctccctggccatcagtggactccagtct
gacgatgagggtgactattattgtgcagcatgggatgacagcctcaatgg
ttatgtcttcggaactgggaccaaggtcaccgtcctgggtcagcccaagg
ctaccccctcggcggccgcagaacaaaaactcatctcagaggaagatctg
tag

SEQ ID NO: 11

ScFv 94 nucleotide sequence
atggccgaggtgcagctggtgcagtctggagctgaggtgaagaagcctgg
ggcctcagtgaaggtctcctgcaaggcttctggttacacctttaccagct
atggtatcagctgggtgcgacaggcccctggacaagggcttgagtggctg
ggctggatcaaccctaacagtggtgacacagtctattcacagaagtttca
gggcagggtcaccatgaccagcgacaagtccgtcagcacagcctacatgg
aactgagcagcctgagatccgacgacacggccgtatattactgtgcctcc
cctgggaaaaattactactacggtatggacgtctggggccaaggcaccct
ggtcaccgtctcctcaggtggcggcggttccggaggtggtggttctggcg
gtggtggcagccagcctgtgctgactcagccaccctcagcgtctgggacc
cccgggcagagggtcaccatctcttgttctggaagcagctccaacatcgg
aagtaatcttatatattggtaccagcagctcccaggaacggcccccaaac
tcctcatctatagtaataatcagcggccctcaggggtccctgaccgattc
tctggctccaagtctggcacctcagcctccctggccatcagtgggctccg
gtccgaggatgaggctgattatttctgttcagcttgggatgacagcctgg
gtggcgaggtcttcggaactgggaccaaggtcaacgtcctaggtcagccc
aaggctgccccctcggcggccgcagaacaaaaactcatctcagaggaaga
tctgtag

SEQ ID NO: 12

ScFv 11 protein sequence
maQVQLVQSGAEVKKPGASVKVSCKASGYTFSGYSTHWLRQVPGQGLEW
IGWDNPSSGDTTYAENFRGRVTLTR2DTSITT
DYLEVRGLRSDDTAVYYCARGGDDYSFDHWGQGTLVTVSSggggsggggs
ggggsSSELTQDPAVSVALGQTVRITCRGD
SLRSYYASWYQQKPGQAPVLVIYGENNRPSGIPDRFSGSSSQDTASLTIT
GAQAEDEADYYCNSRDSSDHLLLFGGGTKL
TVLGqpkatpsaaaeqkliseedl

SEQ ID NO: 13

ScFv 71 protein sequence
maQVQLVQSGAEVKKPGASVKVSCKASGYTFSGYSTHWLRQVPGQGLEW
IGWDNPSSGDTTYAENFRGRVTLTRDTSITT
DYLEVRGLRSDDTAVYYCARGGDDYSFDHWGQGTLVTVSSggggsggggs
ggggsSYELTQPPSASGTTGQRVTISCSGS
SSNIGSNTVNWYQQLPGTAPKLLIYSNNQRPSGVPDRLSGSKSGTSASLA
ISGLQSEDEADYYCAAWDARLTGPLFGGGT
KLSVLRqpkaapsaaaeqkliseedl

SEQ ID NO: 14

ScFv 73 protein sequence
maQVQLVQSGAEVKKPGASVKVSCKASGYTFSGYSTHWLRQVPGQGLEW
IGWDNPSSGDTTYAENFRGRVTLTRDTSITT
DYLEVRGLRSDDTAVYYCARGGDDYSFDHWGQGTLVTVSSggggsggggs
ggggsSYELTQPPSASGTPGQRVTISCSGS
SSNIGSNTVNWYQQLPGTAPKLLIYSNNQRPSGVPDRFSGSKSGTSASLA
ISGLQSEDEADYYCAAWDARLTGPLFGGGT
KLSVLRqpkaapsaaaeqkliseedl

SEQ D NO: 15

ScFv 85 protein sequence
maQVQLVQSGAEVKKPGASVKVSCKASGYTFSGYSTHWLRQVPTGQGLEW
IGWDNPSSGDTTYAENFRGRVTLTRDTSITT
DYLEVRGLRSDDTAVYYCARGGDDYSFDHWGQGTLVTVSSggggsggggs
ggggsSSELTQDPAVSVALGQTVTITCQGG
GLRNYYASWYQQKPGQAPVLLVYGRDNRPSGIPDRFSGSSSGNTASLTIT
GAQAEDEADYYCNSRDSSGNHLVFGGGTKL
TVLGqpkatpsaaaeqkliseedl

SEQ ID NO: 16

ScFv 15 protein sequence
maQVQLVQSGAEVKIKPGASVKVSCKASGYTFSGYSTHWLRQVPGQGLEW
IGWDNPSSGDTTYAENFRGRVTLTRDTSITT
DYLEVRGLRSDDTAVYYCARGGDDYSFDHWGQGTLVTVSSggggsggggs
ggggsQSALTQPASVSGSPGQSITISCTGT
NSDVGIYNLVSWYQQHPGKAPKLMIYDVSNRPSGVSSRFSGSNSGNTATL
TISGLQAEDEADYYCSAHAGDNTQFGGGTK
LTVLSqpkaapsaaaeqkliseedl

SEQ ID NO: 17

ScFv 95 protein sequence
eqldhdyaklackfyfketvimkyllptaaagllllaaqpamaQVQLVQS
GAEVKKPGASVKVSCKASGYTFSGYSTHWL
RQVPGQGLEWIGWDNPSSGDTTYAENFRGRVTLTRDTSITTDYLEVRGLR
SDDTAVYYCARGGDDYSFDHWGQGTLVTVS
SggggsggggsggggsQSALTQPASVSGSPGQSITISCTGTSSDLGGHNF
VSWYQQHPGKAPKLMIYDVFNRPSGVXSRF
XGSKSGNTASLTISGLQAEDEADYFCSSYTITSIVVFGGGTKLTVLGqPk
atpsaaaeqkliseedl

SEQ ID NO: 18

ScFv 84 protein sequence
maQVQLVQSGAEVKKPGASVKVSCKASGYTFSGYSTHWLRQVPGQGLEW
IGWDNPSSGDTTYAENFRGRVTLTRDTSITT
DYLEVRGLRSDDTAVYYCARGGDDYSFDHWGQGTLVTVSSggggsggggs
ggggsQSVLTQPPSVSVAPGKTARIPCGGN
NSGTKSVHWYQQKPGQAPVLVIYDDRVRPSGIPERFSGSNSGDTATLTIS
RVAAGDEADYYCQVSDGSGDPPTWVFGGGT
RLTVLgqpkaapsaaaeqkliseedl

SEQ ID NO: 19

ScFv 10 protein sequence
maQVQLVQSGAEVKKPGSSVKVSCKASGYTFSGYSTHWLRQVPGQGLEWI
GWDNPSSGDTTYAENFRGRVTLTRDTSITT
DYLEVRGLRSDDTAVYYCARGGDDYSFDHWGQGTLVTVSSsgggsggggs
ggggsQTVVTQEPSFSVSPGGTITLTCGLS
SGSVFTSYYPSWYQQTPGQAPRTLIYSTNTRSSGVPDRFSGSILGNKAAL
TITGAQADDESDYYCVLYMGSGIGVFGTGT
KVTVLGqpkaapsaaaeqkliseedl

SEQ ID NO: 20

ScFv 69 protein sequence
maQVQLVQSGAEVKEPGSSVKVSCKASGGTFSNYPISWVRQAPGQGLEW
MGGIIPITNSPGYAQKFQGRVTISADESTGT
VYMELSSLRSEDTAIYYCAKDPNRYESGVLHYWHGLDVWGQGTTVTVSS
ggggsggggsggggsLPVLTQPPSASGTPGQ
TVTLSCSGSSSNIGSNTVNWYQQLPGTAPKLLIYSNNQRPSGVPDRFSAS
KSGTSASLAITGLQAEDEADYFCAAWDDSL
VYVFGTGTKVTVLGqpkaapsaaaeqkliseedl

SEQ ID NO: 21

ScFv 79 protein sequence
maQVQLVQSGAEVKKPGSSVKVSCKASGGTFSSYAISWVRQAPGQGLEW
MGWMNSNTGDTGYAQKFQGRVTMTRNTSTST
AYMELSSLRSEDTAVYYCAKISNYHYYAMDVWGQGTLVTVSSggggsggg
gsggggsLPVLTQPPSASGTSGQTVTISCS
GGSSNIGSHLVTWYQQFPGTAPKVLIHTNDQRPSGVPDRISGSKSGTSAS
LAISGLQSDDEGDYYCAAWDDSLNGYVFGT
GTKVTVLGqpkatpsaaaeqkliseedl

SEQ ID NO: 22

ScFv 94 protein sequence
maEVQLVQSGAEVKKPGASVKVSCKASGYTFTSYGISWVRQALPGQGLEW
LGWINPNSGDTVYSQKFQGRVTMTSDKSVST
AYMELSSLRSDDTAVYYCASPGKNYYYGMDVWGQGTLVTVSSggggsggg
gsggggsQPVLTQPPSASGTPGQRVTISCS
GSSSNIGSNLIYWYQQLPGTAPRLLIYSNNQRPSGVPDRFSGSKSGTSAS
LAISGLRSEDEADYFCSAWDDSLGGEVFGT
GTKVNVLGqpkaapsaaaeqkliseedl

SEQ ID NO: 23

ScFv 11 Heavy chain variable domain protein
sequence
QVQLVQSGAEVKKPGASVKVSCKASGYTFSGYSTHWLRQVPGQGLEWIG
WDNPSSGDTTYAENFRGRVTLTRDTSITT
DYLEVRGLRSDDTAVYYCARGGDDYSFDHWGQGTLVTVSS

SEQ ID NO: 24

ScFv 71 Heavy chain variable domain protein
sequence
QVQLVQSGAEVKKPGASVKVSCKASGYTFSGYSTHWLRQVPGQGLEWIG
WDNPSSGDTTYAENFRGRVTLTRDTSITT
DYLEVRGLRSDDTAVYYCARGGDDYSFDHWGQGTLVTVSS

SEQ ID NO: 25

ScFv 73 Heavy chain variable domain protein
sequence
QVQLVQSGAEVKKPGASVKVSCKASGYTFSGYSTHWLRQVPGQGLEWIG
WDNPSSGDTTYAENFRGRVTLTRDTSITT
DYLEVRGLRSDDTAVYYCARGGDDYSFDHWGQGTLVTVSS

SEQ ID NO: 26

ScFv 85 Heavy chain variable domain protein
sequence
QVQLVQSGAEVKKPGASVKVSCKASGYTFSGYSTHWLRQVPGQGLEWIG
WDNPSSGDTTYAENFRGRVTLTRDTSITT
DYLEVRGLRSDDTAVYYCARGGDDYSFDHWGQGTLVTVSS

SEQ ID NO: 27

ScFv 15 Heavy chain variable domain protein
sequence
QVQLVQSGAEVKKPGASVKVSCKASGYTFSGYSTHWLRQVPGQGLEWIG
WDNPSSGDTTYAENFRGRVTLTRDTSITT
DYLEVRGLRSDDTAVYYCARGGDDYSFDHWGQGTLVTVSS

SEQ ID NO: 28

ScFv 95 Heavy chain variable domain protein
sequence
QVQLVQSGAEVKKPGASVKVSCKASGYTFSGYSTHWLRQVPGQGLEWIG
WDNPSSGDTTYAENFRGRVTLTRDTSITT
DYLEVRGLRSDDTAVYYCARGGDDYSFDHWGQGTLVTVSS

SEQ ID NO: 29

ScFv 84 Heavy chain variable domain protein
sequence
QVQLVQSGAEVKKPGASVKVSCKASGYTFSGYSTHWLRQVPGQGLEWIG
WDNPSSGDTTYAENFRGRVTLTRDTSITT
DYLEVRQLRSDDTAVYYCARGGDDYSFDHWGQGTLVTVSS

SEQ ID NO: 30

ScFv 10 Heavy chain variable domain protein
sequence
QVQLVQSGAEVKKPGSSVKVSCKASGYTFSGYSTHWLRQVPGQGLEWIG
WDNPSSGDTTYAENFRGRVTLTRDTSITT
DYLEVRGLRSDDTAVYYCARGGDDYSFDHWGQGTLVTVSS

SEQ ID NO: 31

ScFv 69 Heavy chain variable domain protein
sequence
QVQLVQSGAEVKEPGSSVKVSCKASGGTFSNYPISWVRQAPGQGLEWMG
GIIPITNSPGYAQKFQGRVTISADESTGT
VYMELSSLRSEDTAIYYCAKDPNRYESGVLHYWHGLDVWGQGTTVTVSS

SEQ ID NO: 32

ScFv 79 Heavy chain variable domain protein
sequence
QVQLVQSGAEVKKPGSSVKVSCKASGGTFSSYAISWVRQAPGQGLEWMG
WMNSNTGDTGYAQKFQGRVTMTRNTSTST
AYMELSSLRSEDTAVYYCAKISNYHYYAMDVWGQGTLVTVSS

SEQ ID NO: 33

ScFv 94 Heavy chain variable domain protein
sequence
EVQLVQSGAEVKKPGASVKVSCKASGYTFTSYGISWVRQAPGQGLEWLG
WINPNSGDTVYSQKFQGRVTMTSDKSVST
AYMELSSLRSDDTAVYYCASPGKNYYYGMDVWGQGTLVTVSS

SEQ ID NO: 34

ScFv 11 Light chain variable domain protein
sequence
SSELTQDPAVSVALGQTVRITCRGDSLRSYYASWYQQKPGQAPVLVIY
GENNRPSGIPDRFSGSSSGDTASLTITGAQAEDEADYYCNSRDSSDHLLL
FGGGTKL

SEQ ID NO: 35

ScFv 71 Light chain variable domain protein
sequence
SYELTQPPSASGTTGQRVTISCSGSSSNIGSNTVNWYQQLPGTAPKLLIY
SNNQRPSGVPDRLSGSKSGTSASLAISGLQSEDEADYYCAAWDARLTGPL
FGGGT
KLSVLR

SEQ ID NO: 36

ScFv 73 Light chain variable domain protein
sequence
SYELTQPPSASGTPGQRVTISCSGSSSNIGSNTVNWYQQLPGTAPKLLIY
SNNQRPSGVPDRFSGSKSGTSASLAISGLQSEDEADYYCAAWDARLTGPL
FGGGT
KLSVLR

SEQ ID NO: 37

ScFv 85 Light chain variable domain protein
sequence
SSELTQDPAVSVALGQTVTITCQGGGLRNYYASWYQQKPGQAPVLLVY
GRDNRPSGIPDRFSGSSSGNTASLTITGAQAEDEADYYCNSRDSSGNHLV
FGGGTKL
TVLG

SEQ ID NO: 38

ScFv 15 Light chain variable domain protein
sequence
QSALTQPASVSGSPGQSITISCTGTNSDVGIYNLVSWYQQHPGKAPKLMIY
DVSNRPSGVSSRFSGSNSGNTATLTISGLQAEDEADYYCSAHAGDNTQFGG
GTKLTVLS

SEQ ID NO: 39

ScFv 95 Light chain variable domain protein
sequence
QSALTQPASVSGSPGQSITISCTGTSSDLGGHNFVSWYQQHPGKAPKLMI
YDVFNRPSGVXSRF
XGSKSGNTASLTISGLQAEDEADYFCSSYTITSIVVFGGGTKLTVLG

SEQ ID NO: 40

ScFv 84 Light chain variable domain protein
sequence
QSVLTQPPSVSVAPGKTARIPCGGNNSGTKSVHWYQQKPGQAPVLVIY
DDRVRPSGIPERFSGSNSGDTATLTISRVAAGDEADYYCQVSDGSGDPPT
WVFGGGTRLTVL

SEQ ID NO: 41

ScFv 10 Light chain variable domain protein
sequence
QTVVTQEPSFSVSPGGTITLTCGLSSGSVFTSYYPSWYQQTPGQALPRTL
IYSTNTRSSGVPDRFSGSILGNKAALTITGAQADDESDYYCVLYMGSGIG
VFGTGTKVTVLG

SEQ ID NO: 42

ScFv 69 Light chain variable domain protein
sequence
LPVLTQPPSASGTPGQ
TVTLSCSGSSSNIGSNTVNWYQQLPGTAPKLLIYSNNQRPSGVPDRFSAS
KSGTSASLAITGLQAEDEADYFCAAWDDSLVYVFGTGTKVTVLG

SEQ ID NO: 43

ScFv 79 Light chain variable domain protein
sequence
LPVLTQPPSASGTSGQTVTISCSGGSSNIGSHLVTWYQQFPGTAPKVLIH
TNDQPSGVPDRISGSKSGTSASLAISGLQSDDEGDYYCAAWDDSLNGYV
FGTGTKVTVLG

SEQ ID NO: 44

ScFv 94 Light chain variable domain protein
sequence
QPVLTQPPSASGTPGQRVTISCSGSSSNIGSNLIYWYQQLPGTAPKLLIY
SNNQRPSGVPDRFSGSKSGTSASLAISGLRSEDEADYFCSAWDDSLGGEV
FGTGTKVNVLG

SEQ ID NO:45

ScFv 11 Heavy chain variable domain nucleotide
sequence
caggtgcagctggtgcagtctggggctgaggtgaagaagcctggggcctc
agtgaaagtctcctgcaaggcttctggatacaccttcagcggctactcta
cacactggctgcgacaggtccctggacagggacttgagtggattggatgg
gacaaccctagtagtggtgacacgacctatgcagagaattttcggggcag
ggtcaccctgaccagggacacgtccatcaccacagattacttggaagtga
ggggtctaagatctgacgacacggccgtctattattattgccagaggcgg
agatgactacagctttgaccattggggtcagggcaccctggtcaccgtct
cctca

SEQ ID NO: 46

ScFv 71 Heavy chain variable domain nucleotide
sequence
caggtgcagctggtgcagtctggggctgaggtgaagaagcctggggcctc
agtgaaagtctcctgcaaggcttctggatacaccttcagcggctactcta
cacactggctgcgacaggtccctggacagggacttgagtggattggatgg
gacaaccctagtagtggtgacacgacctatgcagagaattttcggggcag
ggtcaccctgaccagggacacgtccatcaccacagattacttggaagtga
ggggtctaagatctgacgacacggccgtctattattgtgccagaggcgga
gatgactacagctttgaccattggggtcagggaaccctggtcaccgtctc
ctca

SEQ ID NO:47

ScFv 73 Heavy chain variable domain nucleotide
sequence
caggtgcagctggtgcagtctggggctgaggtgaagaagcctggggcctc
agtgaaagtctcctgcaaggcttctggatacaccttcagcggctactcta
cacactggctgcgacaggtccctggacagggacttgagtggattggatgg
gacaaccctagtagtggtgacacgacctatgcagagaattttcggggcag
ggtcaccctgaccagggacacgtccatcaccacagattacttggaagtga
ggggtctaagatctgacgacacggccgtctattattgtgccagaggcgga
gatgactacagctttgaccattggggtcagggaaccctggtcaccgtctc
ctca

SEQ ID NO: 48

ScFv 85 Heavy chain variable domain nucleotide
sequence
caggtgcagctggtgcagtctggggctgaggtgaagaagcctggggcctc
agtgaaagtctcctgcaaggcttctggatacaccttcagcggctactcta
cacactggctgcgacaggtccctggacagggacttgagtggattggatgg
gacaaccctagtagtggtgacacgacctatgcagagaattttcggggcag
ggtcaccctgaccagggacacgtccatcaccacagattacttggaagtga
ggggtctaagatctgacgacacggccgtctattattgtgccagaggcgga
gatgactacagctttgaccattggggtcagggaaccctggtcaccgtctc
ctca

SEQ ID NO: 49

ScFv 15 Heavy chain variable domain nucleotide
sequence
caggtgcagctggtgcagtctggggctgaggtgaagaagcctggggcctc
agtgaaagtctcctgcaaggcttctggatacaccttcagcggctactcta
cacactggctgcgacaggtccctggacagggacttgagtggattggatgg
gacaaccctagtagtggtgacacgacctatgcagagaattttcggggcag
ggtcaccctgaccagggacacgtccatcaccacagattacttggaagtga
ggggtctaagatctgacgacacggccgtctattattgtgccagaggcgga
gatgactacagctttgaccattggggtcagggaaccctggtcaccgtctc
ctca

SEQ ID NO: 50

ScFv 95 Heavy chain variable domain nucleotide
sequence
caggtgcagctggtgcagtctggggctgaggtgaagaagcctggggcctc
agtgaaagtctcctgcaaggcttctggatacaccttcagcggctactcta
cacactggctgcgacaggtccctggacagggacttgagtggattggatgg
gacaaccctagtagtggtgacacgacctatgcagagaattttcggggcag
ggtcaccctgaccagggacacgtccatcaccacagattacttggaagtga
ggggtctaagatctgacgacacggccgtctattattgtgccagaggcgga
gatgactacagctttgaccattggggtcagggaaccctggtcaccgtctc
ctca

SEQ ID NO: 51

ScFv 84 Heavy chain variable domain nucleotide
sequence
caggtgcagctggtgcagtctggggctgaggtgaagaagcctggggcctc
agtgaaagtctcctgcaaggcttctggatacaccttcagcggctactcta
cacactggctgcgacaggtccctggacagggacttgagtggattggatgg
gacaaccctagtagtggtgacacgacctatgcagagaattttcggggcag
ggtcaccctgaccagggacacgtccatcaccacagattacttggaagtga
ggggtctaagatctgacgacacggccgtctattattgtgccagaggcgga
gatgactacagctttgaccattggggtcagggaaccctggtcaccgtctc
ctca

SEQ ID NO: 52

ScFv 10 Heavy chain variable domain nucleotide
sequence
caggtgcagctggtgcagtctggggctgaggtgaagaagcctgggtcgtc
ggtgaaggtctcctgcaaggcttctggatacaccttcagcggctactcta
cacactggctgcgacaggtccctggacagggacttgagtggattggatgg
gacaaccctagtagtggtgacacgacctatgcagagaattttcggggcag
ggtcaccctgaccagggacacgtccatcaccacagattacttggaagtga
ggggtctaagatctgacgacacggccgtctattattgtgccagaggcgga
gatgactacagctttgaccattggggtcagggcaccctggtcaccgtctc
ctca

SEQ ID NO: 53

ScFv 69 Heavy chain variable domain nucleotide
sequence
caggtgcagctggtgcagtctggggctgaggtgaaggagcctggatcttc
agtgaaagtctcctgtaaggcttctggaggcaccttcagcaattatccta
tcagttgggtgcgacaggcccctggacaagggcttgagtggatgggaggg
atcatccccatcactaattcgccaggctatgcacaaaagttccagggcag
agttacaatttccgcggacgaatcgacgggcacagtctacatggagctga
gcagcctgagatctgaggacacggccatatattactgtgcaaaagatcca
aatcgctatgagagtggtgtactccactattggcacggtttggacgtctg
gggccaagggaccacggtcaccgtctcctca

SEQ ID NO: 54

ScFv 79 Heavy chain variable domain nucleotide
sequence
caggtgcagctggtgcagtctggggctgaggtgaagaagcctgggtcctc
ggtgaaggtctcctgcaaggcttctggaggcaccttcagcagctatgcta
tcagctgggtgcgacaggcccctggacaagggcttgagtggatgggatgg
atgaattctaacactggtgacacaggctatgcacagaagttccagggcag
agtcaccatgaccaggaacacctccacaagcacagcctatatggagctga
gcagcctgagatccgaggacacggccgtctattactgtgcgaaaatctcc
aactaccactattacgctatggacgtctggggccaaggaaccctggtcac
cgtctcctca

SEQ ID NO: 55

ScFv 94 Heavy chain variable domain nucleotide
sequence
gaggtgcagctggtgcagtctggagctgaggtgaagaagcctggggcctc
agtgaaggtctcctgcaaggcttctggttacacctttaccagctatggta
tcagctgggtgcgacaggcccctggacaagggcttgagtggctgggctgg
atcaaccctaacagtggtgacacagtctattcacagaagtttcagggcag
ggtcaccatgaccagcgacaagtccgtcagcacagcctacatggaactga
gcagcctgagatccgacgacacggccgtatattactgtgcctcccctggg
aaaaattactactacggtatggacgtctggggccaaggcaccctggtcac
cgtctcctca

SEQ ID NO: 56

ScFv 11 Light chain variable domain nucleotide
sequence
tcttctgagctgactcaggacccagctgtgtctgtggccttgggacagac
agtcaggatcacatgccgaggagacagcctcagaagttattatgcaagct
ggtaccaacagaagccaggacaggcccctgtacttgtcatctatggtgaa
aacaaccgaccctcagggatcccagaccgattctctggctccagctcagg
agacacagcttccttgaccatcactggggctcaggcggaagatgaggctg
actattactgtaactcccgggacagcagtgatcaccttctcctattcggt
ggagggaccaagttgaccgtcctaggt

SEQ ID NO: 57

ScFv 71 Light chain variable domain nucleotide
sequence
tcctatgagctgactcagccaccatcagcgtctgggaccaccgggcagag
ggtcaccatctcttgttctggaagcagctccaacatcggaagtaatactg
taaactggtaccagcagctcccaggaacggcccccaaactcctcatctat
agtaataatcagcggccctcaggggtccctgaccgattatctggctccaa
gtctggcacctcagcctccctggccatcagtggactccagtctgaagatg
aggccgattattactgtgctgcgtgggatgcccgcctgactggtcccctc
ttcggcggggggaccaagctaagcgtcctacgt

SEQ ID NO: 58

ScFv 73 Light chain variable domain nucleotide
sequence
tcctatgagctgactcagccaccctcagcgtctgggacccccgggcagag
ggtcaccatctcttgttctggaagcagctccaacatcggaagtaatactg
taaactggtaccagcagctcccaggaacggcccccaaactcctcatctat
agtaataatcagcggccctcaggggtccctgaccgattctctggctccaa
gtctggcacctcagcctccctggccatcagtggactccagtctgaagatg
aggccgattattactgtgctgcgtgggatgcccgcctgactggtcccctc
ttcggcggggggaccaagctaagcgtcctacgt

SEQ ID NO: 59

ScFv 85 Light chain variable domain nucleotide
sequence
tcttctgagctgactcaggaccctgctgtgtctgtggccttggggcagac
agtcacgatcacatgtcaaggaggcggcctcagaaattattatgcaagtt
ggtaccaacagaagccgggacaggcccctgtccttctcgtctatggaaga
gacaaccggccctcagggatcccagaccgattctctggctccagctcagg
aaacacagcttccttgaccatcactggggctcaggcggaagatgaggctg
actattactgtaactcccgggacagcagtggtaaccatctggtgttcggc
ggagggaccaagctgaccgtcctaggt

SEQ ID NO: 60

ScFv 15 Light chain variable domain nucleotide
sequence
cagtctgccctgactcagcctgcctccgtgtctgggtctcctggacagtc
gatcaccatctcctgcactggaaccaacagtgatgttggaatttataacc
ttgtctcctggtaccaacagcacccaggcaaagcccccaaactcatgatt
tatgatgtcagtaatcggccctcaggggtttctagtcgcttctctggctc
caactctgggaacacggccaccctgaccatctctgggctccaggctgaag
atgaggctgattattattgcagcgcacatgcaggcgacaacacccaattc
ggcggagggaccaagctgaccgtcctaagt

SEQ ID NO: 61

ScFv 95 Light chain variable domain nucleotide
sequence
cagtctgccctgactcagcctgcctccgtgtctgggtctcctggacagtc
gatcaccatctcctgcactggaaccagcagtgaccttggtggtcacaact
ttgtctcctggtaccaacagcacccaggcaaagcccccaaactcatgatt
tatgatgtctttaatcggccctcaggggtttntagtcgnttctntggctc
caagtctggcaacacggcctccctgaccatctctgggctccaggctgagg
acgaggctgattatttctgcagctcatatacaatcaccagcatcgtggtc
ttcggcggagggaccaagctgaccgtcctaggt

SEQ ID NO: 62

ScFv 84 Light chain variable domain nucleotide
sequence
cagtctgtgctgactcagccaccctcagtgtcagtggccccaggaaagac
ggccaggattccctgtgggggaaacaacagtggaactaaaagtgtgcact
ggtaccagcagaagccaggccaggcccctgtgctggtcatctatgatgat
agagtccggccctcagggatccctgagcgattctctggctccaactctgg
ggacacggccaccctgaccatcagcagggtcgcagccggggatgaggccg
actattactgtcaggtgtcggatggtagtggtgatcctcccacttgggtg
ttcggcggagggaccaggctgaccgtcctaggt

SEQ ID NO: 63

ScFv 10 Light chain variable domain nucleotide
sequence
cagactgtggtgactcaggagccatcgttctcagtgtcccctggagggac
catcacactcacttgtggcttgagctctggctcagtctttactagttact
accccagctggtaccagcagaccccaggccaggctccacgcacgctcatc
tacagcacaaacactcgctcttctggggtccctgatcgcttctctggctc
catccttgggaacaaagctgccctcaccatcacgggggcccaggcagatg
atgaatctgattattactgtgtcctgtatatgggtagtggcattggggtc
ttcggaactgggaccaaggtcaccgtcctaggt

SEQ ID NO: 64

ScFv 69 Light chain variable domain nucleotide
sequence
ctgcctgtgctgactcagccaccctcagcgtcggggacccccgggcagac
ggttaccctctcttgttctggaagcagctccaacatcggaagtaatactg
taaactggaccagcagctcccaggaacggcccccaaactcctcatctata
gtaataatcagcggccctcaggggtccctgaccgattctctgcctccaag
tctggcacctcagcctccctggccatcactgggctccaggctgaggatga
ggctgattatttctgtgcagcatgggatgacagcctggtttatgtcttcg
gaactgggaccaaggtcaccgtcctaggt

SEQ ID NO: 65

ScFv 79 Light chain variable domain nucleotide
sequence
ctgcctgtgctgactcagccaccctcagcgtctgggacctccgggcagac
ggtcaccatctcctgttctggagggagctccaacatcggaagtcatcttg
taacctggtaccagcagtttccagggacggcccccaaagtcctcatacat
actaatgatcagcgaccctctggggtccctgaccgaatctctggctccaa
gtctggcacctcagcctccctggccatcagtggactccagtctgacgatg
agggtgactattattgtgcagcatgggatgacagcctcaatggttatgtc
ttcggaactgggaccaaggtcaccgtcctgggt

SEQ ID NO: 66

ScFv 94 Light chain variable domain nucleotide
sequence
cagcctgtgctgactcagccaccctcagcgtctgggaccccegggcagag
ggtcaccatctcttgttctggaagcagctccaacatcggaagtaatctta
tatattggtaccagcagctcccaggaacggcccccaaactcctcatctat
agtaataatcagcggccctcaggggtccctgaccgattctctggctccaa
gtctggcacctcagcctccctggccatcagtgggctccggtccgaggatg
aggctgattatttctgttcagcttgggatgacagcctgggtggcgaggtc
ttcggaactgggaccaaggtcaacgtcctaggt

SEQ ID NO: 67

complement to ScFv 71 nucleotide sequence
ctacagatcttcctctgagatgagtttttgttctgcggccgccgaggggg
cggccttgggctgacgtaggacgcttagcttggtccccccgccgaagagg
ggaccagtcaggcgggcatcccacgcagcacagtaataatcggcctcatc
ttcagactggagtccactgatggccagggaggctgaggtgccagacttgg
agccagataatcggtcagggacccctgagggccgctgattattactatag
atgaggagtttgggggccgttcctgggagctgctggtaccagtttacagt
attacttccgatgttggagctgcttccagaacaagagatggtgaccctct
gcccggtggtcccagacgctgatggtggctgagtcagctcataggagctg
ccaccaccgccagaaccaccacctccggaaccgccgccacctgaggagac
ggtgaccagggttccctgaccccaatggtcaaagctgtagtcatctccgc
ctctggcacaataatagacggccgtgtcgtcagatcttagacccctcact
tccaagtaatctgtggtgatggacgtgtccctggtcagggtgaccctgcc
ccgaaaattctctgcataggtcgtgtcaccactactagggttgtcccatc
caatccactcaagtccctgtccagggacctgtcgcagccagtgtgtagag
tagccgctgaaggtgtatccagaagccttgcaggagactttcactgaggc
cccaggcttcttcacctcagccccagactgcaccagctgcacctgggcc
at

What is claimed is:

1. An isolated human anti-West Nile Virus envelope protein (WNE) antibody or an antigen-binding portion thereof.

2. The human antibody or antigen-binding portion according to claim 1, wherein said antibody or portion possesses at least one of the following properties:
(a) binds to a domain I/domain II region of a West Nile Virus E protein;
(b) binds to the ectodomain of a West Nile Virus E protein;
(c) binds to a West Nile Virus E protein (WNE) with a K_{D} of 6.0×10⁻⁸ M or less;
(d) has an off rate (k_{off}) for a WNE of 7.0×10⁻³ s⁻¹ or smaller;
(e) inhibits fusion of a West Nile Virus with a target cell membrane; or
(f) binds a Dengue virus E protein and the E protein of at least one flavivirus of the Japanese Encephalitis Antigenic Complex.

3. The human antibody or portion according to claim 1 or claim 2, wherein the Japanese Encephalitis Antigenic Complex flavivirus is selected from the group consisting of: West Nile virus, St. Louis Encephalitis virus, Murray Valley Encephalitis virus, Japanese Encephalitis virus, and Kunjin virus.

4. The human antibody or portion according to claim 2, wherein the Dengue virus is selected from the group consisting of: Dengue-1, Dengue-2, Dengue-3, and Dengue-4.

5. The human antibody or portion according to claim 2, wherein said antibody or portion binds WNE with a K_{D} of 6.0×10⁻⁸ M or less and prevents, inhibits, or treats a Japanese Encephalitis Antigenic Complex flavivirus infection or disease.

6. A humanized, chimeric or human anti-WNE antibody or antigen-binding portion thereof, wherein the antibody or portion thereof has at least one property selected from the group consisting of:
(a) cross-competes for binding to WNE with an antibody selected from the group consisting of 11, 71, 73, 85, 15, 95, 84, 10, 69, 79, and 94;
(b) competes for binding to WNE with an antibody selected from the group consisting of 11, 71, 73, 85, 15, 95, 84, 10, 69, 79, and 94;
(c) binds to the same epitope of WNE as an antibody selected from the group consisting of 11, 71, 73, 85, 15, 95, 84, 10, 69, 79, and 94;
(d) binds to WNE with substantially the same K_{D} as an antibody selected from the group consisting of 11, 71, 73, 85, 15, 95, 84, 10, 69, 79, and 94; and
(e) binds to WNE with substantially the same off rate as an antibody selected from the group consisting of 11, 71, 73, 85, 15, 95, 84, 10, 69, 79, and 94.

7. An isolated anti-WNE antibody, wherein the antibody is selected from the group consisting of:
(a) an antibody comprising the heavy chain amino acid sequence set forth in SEQ ID NO: 23 and the light chain amino acid sequence set forth in SEQ ID NO: 34;
(b) an antibody comprising the heavy chain amino acid sequence set forth in SEQ ID NO: 24 and the light chain amino acid sequence set forth in SEQ ID NO: 35;
(c) an antibody comprising the heavy chain amino acid sequence set forth in SEQ ID NO: 25 and the light chain amino acid sequence set forth in SEQ ID NO: 36;
(d) an antibody comprising the heavy chain amino acid sequence set forth in SEQ ID NO: 26 and the light chain amino acid sequence set forth in SEQ ID NO: 37;
(e) an antibody comprising the heavy chain amino acid sequence set forth in SEQ ID NO: 27 and the light chain amino acid sequence set forth in SEQ ID NO: 38;
(f) an antibody comprising the heavy chain amino acid sequence set forth in SEQ ID NO: 28 and the light chain amino acid sequence set forth in SEQ ID NO: 39;
(g) an antibody comprising the heavy chain amino acid sequence set forth in SEQ ID NO: 29 and the light chain amino acid sequence set forth in SEQ ID NO: 40;
(h) an antibody comprising the heavy chain amino acid sequence set forth in SEQ ID NO: 30 and the light chain amino acid sequence set forth in SEQ ID NO: 41;
(i) an antibody comprising the heavy chain having the amino acid sequence set forth in SEQ ID NO: 31 and the light chain having the amino acid sequence set forth in SEQ ID NO: 42;
(j) an antibody comprising the heavy chain amino acid sequence set forth in SEQ ID NO: 32 and the light chain amino acid sequence set forth in SEQ ID NO: 43;
(k) an antibody comprising the heavy chain amino acid sequence set forth in SEQ ID NO: 33 and the light chain amino acid sequence set forth in SEQ ID NO: 44;

8. The human antibody or antigen-binding portion according to claim 1, wherein said antibody or antigen-binding portion comprises:
(a) heavy chain CDR1, CDR2 and CDR3 sequences independently selected from the heavy chain CDR1, CDR2 and CDR3, respectively, of an antibody selected from the group consisting of antibodies 11, 71, 73, 85, 15, 95, 84, 10, 69, 79, and 94;
(b) light chain CDR1, CDR2 and CDR3 sequences independently selected from the light chain CDR1, CDR2 and CDR3, respectively, of an antibody selected from the group consisting of antibodies 11, 71, 73, 85, 15, 95, 84, 10, 69, 79, and 94; or
(c) both (a) and (b).

9. The human antibody or antigen-binding portion according to claim 1, wherein said antibody or portion comprises a heavy chain that utilizes a human V_{H} 1 family gene.

10. The human antibody or an antigen-binding portion thereof according to claim 9, wherein said antibody or portion comprises a light chain that utilizes a human V lambda 1 family gene, human V lambda 2 family gene, human V lambda 3 family gene, or a human V lambda 8 family gene.

11. The human antibody according to claim 1 wherein the V_{L} and V_{H} domains are at least 90% identical in amino acid sequence to the V_{L} and V_{H} domains, respectively, of an antibody selected from the group consisting of: antibodies 11, 71, 73, 85, 15, 95, 84, 10, 69, 79, or 94.

12. The human antibody according to claim 1, wherein the antibody comprises:
(a) a heavy chain amino acid sequence that is at least 90% identical to the heavy chain amino acid sequence of antibody 11, 71, 73, 85, 15, 95, 84, 10, 69, 79, and 94;
(b) a light chain amino acid sequence that is at least 90% identical to the light chain amino acid sequence of antibody 11, 71, 73, 85, 15, 95, 84, 10, 69, 79, and 94; or
(c) both (a) and (b).

13. An isolated anti-WNE antibody or an antigen-binding, wherein:
(a) the heavy chain comprises the heavy chain CDR1, CDR2 and CDR3 amino acid sequences of an antibody selected from the group consisting of: 11, 71, 73, 85, 15, 95, 84, 10, 69, 79, and 94;
(b) the light chain comprises the light chain CDR1, CDR2 and CDR3 amino acid sequences of an antibody selected from the group consisting of 11, 71, 73, 85, 15, 95, 84, 10, 69, 79, and 94;
(c) the antibody comprises a heavy chain of (a) and a light chain of (b); or
(d) the antibody of (c) wherein the heavy chain and light chain CDR amino acid sequences are selected from the same antibody selected from the group consisting of: 11, 71, 73, 85, 15, 95, 84, 10, 69, 79, and 94.

14. The human antibody or portion according to claim 11:
(a) wherein said heavy chain comprises the amino acid sequence of the variable domain of the heavy chain of an antibody selected from the group consisting of: 11, 71, 73, 85, 15, 95, 84, 10, 69, 79, and 94;
(b) wherein said light chain comprises the amino acid sequence of the variable domain of the light chain of an antibody selected from the group consisting of: 11, 71, 73, 85, 15, 95, 84, 10, 69, 79, and 94;
(c) wherein said antibody or portion comprises both of said variable domains; or
(d) wherein said antibody or portion comprises variable domain sequences from the same antibody selected from the group consisting of: 11, 71, 73, 85, 15, 95, 84, 10, 69, 79, and 94.

15. A isolated anti-WNE antibody or an antigen-binding portion thereof, wherein the antibody comprises one or more of an FR1, FR2, FR3 or FR4 amino acid sequence of an antibody selected from the group consisting of: antibody 11, 71, 73, 85, 15, 95, 84, 10, 69, 79, and 94.

16. A pharmaceutical composition comprising the antibody or antigen-binding portion according to any one of claims 1 to 15 and a pharmaceutically acceptable carrier.

17. The pharmaceutical composition according to claim 16, further comprising one or more additional antibodies or antigen-binding portions that specifically bind a Japanese Encephalitis Antigenic Complex flavivirus E protein.

18. The pharmaceutical composition according to claim 16 or claim 17, wherein the composition is in injectable form.

19. A method for treating, inhibiting, or preventing a flavivirus infection or disease in a subject in need thereof, comprising administering to said subject an antibody or antigen-binding portion according to any one of claims 1 to 15 or a pharmaceutical composition according to any one of claims 16 to 18, wherein said antibody or antigen-binding portion treats, inhibits, or prevents the flavivirus infection or disease in the subject.

20. The method of claim 19, wherein the antibody, antigen-binding portion, or pharmaceutical composition is administered prior to infection of the subject with said flavivirus.

21. The method of claim 19, wherein the antibody, antigen-binding portion, or pharmaceutical composition is administered after infection of the subject with said flavivirus.

22. The method according to any one of claims 19-21, wherein said flavivirus is a Japanese Encephalitis Antigenic Complex virus or a Dengue virus.

23. The method according to any one of claims 19-21, wherein said Japanese Encephalitis Antigenic Complex virus is selected from the group consisting of: West Nile virus, St. Louis Encephalitis virus, Murray Valley Encephalitis virus, Japanese Encephalitis virus, and Kunjin virus.

24. The method of claim 19, further comprising administering one or more therapeutic, prophylactic, or diagnostic agents in combination with said antibody, said antigen-binding portion, or said pharmaceutical composition.

25. An isolated cell line that produces the antibody or antigen-binding portion according to any one of claims 1 to 15 or the heavy chain or light chain of said antibody or said portion.

26. An isolated nucleic acid molecule comprising a nucleotide sequence that encodes the heavy chain or an antigen-binding portion thereof or the light chain or an antigen-binding portion thereof of an antibody according to any one of claims 1 to 15.

27. A vector comprising the nucleic acid molecule according to claim 26, wherein the vector optionally comprises an expression control sequence operably linked to the nucleic acid molecule.

28. A host cell comprising the vector according to claim 27 or the nucleic acid molecule according to claim 26.

29. A method for producing an anti-WNE antibody or antigen-binding portion thereof, comprising culturing the host cell according to claim 28 or the cell line according to claim 25 under suitable conditions and recovering said antibody or antigen-binding portion.

30. A non-human transgenic animal or transgenic plant comprising the nucleic acid according to claim 26, wherein the non-human transgenic animal or transgenic plant expresses said nucleic acid.

31. A method for isolating an anti-WNE antibody or antigen-binding portion thereof, comprising isolating the antibody from the non-human transgenic animal or transgenic plant according to claim 30.

32. A method for treating a subject in need thereof with an antibody or antigen-binding portion according to any one of claims 1-15, comprising:
(a) administering an effective amount of an isolated nucleic acid molecule encoding the heavy chain or the antigen-binding portion thereof of an antibody according to any one of claims 1 to 15, an isolated nucleic acid molecule encoding the light chain or the antigen-binding portion thereof of an antibody according to any one of claims 1 to 15, or both the nucleic acid molecules encoding the light chain and the heavy chain or antigen-binding portions thereof of an antibody according to any one of claims 1 to 15; and
(b) expressing the nucleic acid molecule.

33. A method for making a human anti-WNE antibody, comprising:
(a) immunizing a non-human transgenic animal that is capable of producing human antibodies with an immunogen selected from the group consisting of: full length WNE; the ectodomain of WNE; domain I/II of WNE; domain III of WNE, an immunogenic portion of WNE or a cell or tissue expressing WNE;
(b) allowing the non-human transgenic animal to mount an immune response to the WNE immunogen; and
(c) isolating B lymphocytes from the non-human transgenic animal.

34. An isolated antibody produced by the method according to claim 29.

35. A method for detecting a Japanese Encephalitis Antigenic Complex flavivirus infection, comprising contacting a sample from a subject suspected of having said infection with the antibody or antigen-binding portion thereof according to any one of claims 1-14.

36. A method of identifying a protective anti-WNE antibody, comprising:
(a) passively immunizing a non-human animal with an anti-WNE antibody;
(b) challenging the immunized non-human animal produced in (a) with West Nile virus; and
(c) identifying an antibody that confers protection against West Nile virus infection or disease.

37. A method for producing a protective anti-WNE antibody, comprising immunizing a non-human animal with WNE peptide 29 or WNE 39 and recovering the antibody.


SEQUENCE LISTING (140 sequences)

SEQ ID NO: 1 - DNA, 795 bp, Homo sapiens
atggcccaggtgcagctggtgcagtctggggctgaggtgaagaagcctggggcctcagtgaaagtctcctgcaaggcttctggatacaccttcagcggctactctacacactggctgcgacaggtccctggacagggacttgagtggattggatgggacaaccctagtagtggtgacacgacctatgcagagaattttcggggcagggtcaccctgaccagggacacgtccatcaccacagattacttggaagtgaggggtctaagatctgacgacacggccgtctattattgtgccagaggcggagatgactacagctttgaccattggggtcagggcaccctggtcaccgtctcctcaggtggcggcggttccggaggtggtggttctggcggtggtggcagctcttctgagctgactcaggacccagctgtgtctgtggccttgggacagacagtcaggatcacatgccgaggagacagcctcagaagttattatgcaagctggtaccaacagaagccaggacaggcccctgtacttgtcatctatggtgaaaacaaccgaccctcagggatcccagaccgattctctggctccagctcaggagacacagcttccttgaccatcactggggctcaggcggaagatgaggctgactattactgtaactcccgggacagcagtgatcaccttctcctattcggtggagggaccaagttgaccgtcctaggtcagcccaaggctaccccctcggcggccgcagaacaaaaactcatctcagaggaagatctgtag

SEQ ID NO: 2 - DNA, 801 bp, Homo sapiens
atggcccaggtgcagctggtgcagtctggggctgaggtgaagaagcctggggcctcagtgaaagtctcctgcaaggcttctggatacaccttcagcggctactctacacactggctgcgacaggtccctggacagggacttgagtggattggatgggacaaccctagtagtggtgacacgacctatgcagagaattttcggggcagggtcaccctgaccagggacacgtccatcaccacagattacttggaagtgaggggtctaagatctgacgacacggccgtctattattgtgccagaggcggagatgactacagctttgaccattggggtcagggaaccctggtcaccgtctcctcaggtggcggcggttccggaggtggtggttctggcggtggtggcagctcctatgagctgactcagccaccatcagcgtctgggaccaccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattatctggctccaagtctggcacctcagcctccctggccatcagtggactccagtctgaagatgaggccgattattactgtgctgcgtgggatgcccgcctgactggtcccctcttcggcggggggaccaagctaagcgtcctacgtcagcccaaggccgccccctcggcggccgcagaacaaaaactcatctcagaggaagatctgtag

SEQ ID NO: 3 - DNA, 801 bp, Homo sapiens
atggcccaggtgcagctggtgcagtctggggctgaggtgaagaagcctggggcctcagtgaaagtctcctgcaaggcttctggatacaccttcagcggctactctacacactggctgcgacaggtccctggacagggacttgagtggattggatgggacaaccctagtagtggtgacacgacctatgcagagaattttcggggcagggtcaccctgaccagggacacgtccatcaccacagattacttggaagtgaggggtctaagatctgacgacacggccgtctattattgtgccagaggcggagatgactacagctttgaccattggggtcagggaaccctggtcaccgtctcctcaggtggcggcggttccggaggtggtggttctggcggtggtggcagctcctatgagctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtggactccagtctgaagatgaggccgattattactgtgctgcgtgggatgcccgcctgactggtcccctcttcggcggggggaccaagctaagcgtcctacgtcagcccaaggccgccccctcggcggccgcagaacaaaaactcatctcagaggaagatctgtag

SEQ ID NO: 4 - DNA, 795 bp, Homo sapiens
atggcccaggtgcagctggtgcagtctggggctgaggtgaagaagcctggggcctcagtgaaagtctcctgcaaggcttctggatacaccttcagcggctactctacacactggctgcgacaggtccctggacagggacttgagtggattggatgggacaaccctagtagtggtgacacgacctatgcagagaattttcggggcagggtcaccctgaccagggacacgtccatcaccacagattacttggaagtgaggggtctaagatctgacgacacggccgtctattattgtgccagaggcggagatgactacagctttgaccattggggtcagggaaccctggtcaccgtctcctcaggtggcggcggttccggaggtggtggttctggcggtggtggcagctcttctgagctgactcaggaccctgctgtgtctgtggccttggggcagacagtcacgatcacatgtcaaggaggcggcctcagaaattattatgcaagttggtaccaacagaagccgggacaggcccctgtccttctcgtctatggaagagacaaccggccctcagggatcccagaccgattctctggctccagctcaggaaacacagcttccttgaccatcactggggctcaggcggaagatgaggctgactattactgtaactcccgggacagcagtggtaaccatctggtgttcggcggagggaccaagctgaccgtcctaggtcagcccaaggccaccccctcggcggccgcagaacaaaaactcatctcagaggaagatctgtag

SEQ ID NO: 5 - DNA, 798 bp, Homo sapiens
atggcccaggtgcagctggtgcagtctggggctgaggtgaagaagcctggggcctcagtgaaagtctcctgcaaggcttctggatacaccttcagcggctactctacacactggctgcgacaggtccctggacagggacttgagtggattggatgggacaaccctagtagtggtgacacgacctatgcagagaattttcggggcagggtcaccctgaccagggacacgtccatcaccacagattacttggaagtgaggggtctaagatctgacgacacggccgtctattattgtgccagaggcggagatgactacagctttgaccattggggtcagggaaccctggtcaccgtctcctcaggtggcggcggttccggaggtggtggttctggcggtggtggcagccagtctgccctgactcagcctgcctccgtgtctgggtctcctggacagtcgatcaccatctcctgcactggaaccaacagtgatgttggaatttataaccttgtctcctggtaccaacagcacccaggcaaagcccccaaactcatgatttatgatgtcagtaatcggccctcaggggtttctagtcgcttctctggctccaactctgggaacacggccaccctgaccatctctgggctccaggctgaagatgaggctgattattattgcagcgcacatgcaggcgacaacacccaattcggcggagggaccaagctgaccgtcctaagtcagcccaaggctgccccctcggcggccgcagaacaaaaactcatctcagaggaagatctgtag

SEQ ID NO: 6 - DNA, 925 bp, Homo sapiens
  modified_base (711): a, c, g, t, unknown, or other
ggaacagcttgaccatgattacgccaagcttgcatgcaaattctatttcaaggagacagtcataatgaaatacctattgcctacggcagccgctggattgttattactcgcggcccagccggccatggcccaggtgcagctggtgcagtctggggctgaggtgaagaagcctggggcctcagtgaaagtctcctgcaaggcttctggatacaccttcagcggctactctacacactggctgcgacaggtccctggacagggacttgagtggattggatgggacaaccctagtagtggtgacacgacctatgcagagaattttcggggcagggtcaccctgaccagggacacgtccatcaccacagattacttggaagtgaggggtctaagatctgacgacacggccgtctattattgtgccagaggcggagatgactacagctttgaccattggggtcagggaaccctggtcaccgtctcctcaggtggcggcggttccggaggtggtggttctggcggtggtggcagccagtctgccctgactcagcctgcctccgtgtctgggtctcctggacagtcgatcaccatctcctgcactggaaccagcagtgaccttggtggtcacaactttgtctcctggtaccaacagcacccaggcaaagcccccaaactcatgatttatgatgtctttaatcggccctcaggggtttntagtcgnttctntggctccaagtctggcaacacggcctccctgaccatctctgggctccaggctgaggacgaggctgattatttctgcagctcatatacaatcaccagcatcgtggtcttcggcggagggaccaagctgaccgtcctaggtcagcccaaggccaccccctcggcggccgcagaacaaaaactcatctcagaggaagatctgtag

SEQ ID NO: 7 - DNA, 801 bp, Homo sapiens
atggcccaggtgcagctggtgcagtctggggctgaggtgaagaagcctggggcctcagtgaaagtctcctgcaaggcttctggatacaccttcagcggctactctacacactggctgcgacaggtccctggacagggacttgagtggattggatgggacaaccctagtagtggtgacacgacctatgcagagaattttcggggcagggtcaccctgaccagggacacgtccatcaccacagattacttggaagtgaggggtctaagatctgacgacacggccgtctattattgtgccagaggcggagatgactacagctttgaccattggggtcagggaaccctggtcaccgtctcctcaggtggcggcggttccggaggtggtggttctggcggtggtggcagccagtctgtgctgactcagccaccctcagtgtcagtggccccaggaaagacggccaggattccctgtgggggaaacaacagtggaactaaaagtgtgcactggtaccagcagaagccaggccaggcccctgtgctggtcatctatgatgatagagtccggccctcagggatccctgagcgattctctggctccaactctggggacacggccaccctgaccatcagcagggtcgcagccggggatgaggccgactattactgtcaggtgtcggatggtagtggtgatcctcccacttgggtgttcggcggagggaccaggctgaccgtcctaggtcagcccaaggctgccccctcggcggccgcagaacaaaaactcatctcagaggaagatctgtag

SEQ ID NO: 8 - DNA, 801 bp, Homo sapiens
atggcccaggtgcagctggtgcagtctggggctgaggtgaagaagcctgggtcgtcggtgaaggtctcctgcaaggcttctggatacaccttcagcggctactctacacactggctgcgacaggtccctggacagggacttgagtggattggatgggacaaccctagtagtggtgacacgacctatgcagagaattttcggggcagggtcaccctgaccagggacacgtccatcaccacagattacttggaagtgaggggtctaagatctgacgacacggccgtctattattgtgccagaggcggagatgactacagctttgaccattggggtcagggcaccctggtcaccgtctcctcaagtggcggcggttccggaggtggtggttctggcggtggtggcagccagactgtggtgactcaggagccatcgttctcagtgtcccctggagggaccatcacactcacttgtggcttgagctctggctcagtctttactagttactaccccagctggtaccagcagaccccaggccaggctccacgcacgctcatctacagcacaaacactcgctcttctggggtccctgatcgcttctctggctccatccttgggaacaaagctgccctcaccatcacgggggcccaggcagatgatgaatctgattattactgtgtcctgtatatgggtagtggcattggggtcttcggaactgggaccaaggtcaccgtcctaggtcagcccaaggctgccccctcggcggccgcagaacaaaaactcatctcagaggaagatctgtag

SEQ ID NO: 9 - DNA, 825 bp, Homo sapiens
atggcccaggtgcagctggtgcagtctggggctgaggtgaaggagcctggatcttcagtgaaagtctcctgtaaggcttctggaggcaccttcagcaattatcctatcagttgggtgcgacaggcccctggacaagggcttgagtggatgggagggatcatccccatcactaattcgccaggctatgcacaaaagttccagggcagagttacaatttccgcggacgaatcgacgggcacagtctacatggagctgagcagcctgagatctgaggacacggccatatattactgtgcaaaagatccaaatcgctatgagagtggtgtactccactattggcacggtttggacgtctggggccaagggaccacggtcaccgtctcctcaggtggcggcggttccggaggtggtggttctggcggtggtggcagcctgcctgtgctgactcagccaccctcagcgtcggggacccccgggcagacggttaccctctcttgttctggaagcagctccaacatcggaagtaatactgtaaactggtaccagcagctcccaggaacggcccccaaactcctcatctatagtaataatcagcggccctcaggggtccctgaccgattctctgcctccaagtctggcacctcagcctccctggccatcactgggctccaggctgaggatgaggctgattatttctgtgcagcatgggatgacagcctggtttatgtcttcggaactgggaccaaggtcaccgtcctaggtcagcccaaggctgccccctcggcggccgcagaacaaaaactcatctcagaggaagatctgtag

SEQ ID NO: 10 - DNA, 807 bp, Homo sapiens
atggcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggatggatgaattctaacactggtgacacaggctatgcacagaagttccagggcagagtcaccatgaccaggaacacctccacaagcacagcctatatggagctgagcagcctgagatccgaggacacggccgtctattactgtgcgaaaatctccaactaccactattacgctatggacgtctggggccaaggaaccctggtcaccgtctcctcaggtggcggcggttccggaggtggtggttctggcggtggtggcagcctgcctgtgctgactcagccaccctcagcgtctgggacctccgggcagacggtcaccatctcctgttctggagggagctccaacatcggaagtcatcttgtaacctggtaccagcagtttccagggacggcccccaaagtcctcatacatactaatgatcagcgaccctctggggtccctgaccgaatctctggctccaagtctggcacctcagcctccctggccatcagtggactccagtctgacgatgagggtgactattattgtgcagcatgggatgacagcctcaatggttatgtcttcggaactgggaccaaggtcaccgtcctgggtcagcccaaggctaccccctcggcggccgcagaacaaaaactcatctcagaggaagatctgtag

SEQ ID NO: 11 - DNA, 807 bp, Homo sapiens
atggccgaggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggctgggctggatcaaccctaacagtggtgacacagtctattcacagaagtttcagggcagggtcaccatgaccagcgacaagtccgtcagcacagcctacatggaactgagcagcctgagatccgacgacacggccgtatattactgtgcctcccctgggaaaaattactactacggtatggacgtctggggccaaggcaccctggtcaccgtctcctcaggtggcggcggttccggaggtggtggttctggcggtggtggcagccagcctgtgctgactcagccaccctcagcgtctgggacccccgggcagagggtcaccatctcttgttctggaagcagctccaacatcggaagtaatcttatatattggtaccagcagctcccaggaacggcccccaaactcctcatctatagtaataatcagcggccctcaggggtccctgaccgattctctggctccaagtctggcacctcagcctccctggccatcagtgggctccggtccgaggatgaggctgattatttctgttcagcttgggatgacagcctgggtggcgaggtcttcggaactgggaccaaggtcaacgtcctaggtcagcccaaggctgccccctcggcggccgcagaacaaaaactcatctcagaggaagatctgtag

SEQ ID NO: 12 - protein, 264 aa, Homo sapiens
MAQVQLVQSGAEVKKPGASVKVSCKASGYTFSGYSTHWLRQVPGQGLEWIGWDNPSSGDTTYAENFRGRVTLTRDTSITTDYLEVRGLRSDDTAVYYCARGGDDYSFDHWGQGTLVTVSSGGGGSGGGGSGGGGSSSELTQDPAVSVALGQTVRITCRGDSLRSYYASWYQQKPGQAPVLVIYGENNRPSGIPDRFSGSSSGDTASLTITGAQAEDEADYYCNSRDSSDHLLLFGGGTKLTVLGQPKATPSAAAEQKLISEEDL

SEQ ID NO: 13 - protein, 266 aa, Homo sapiens
MAQVQLVQSGAEVKKPGASVKVSCKASGYTFSGYSTHWLRQVPGQGLEWIGWDNPSSGDTTYAENFRGRVTLTRDTSITTDYLEVRGLRSDDTAVYYCARGGDDYSFDHWGQGTLVTVSSGGGGSGGGGSGGGGSSYELTQPPSASGTTGQRVTISCSGSSSNIGSNTVNWYQQLPGTAPKLLIYSNNQRPSGVPDRLSGSKSGTSASLAISGLQSEDEADYYCAAWDARLTGPLFGGGTKLSVLRQPKAAPSAAAEQKLISEEDL

SEQ ID NO: 14 - protein, 266 aa, Homo sapiens
MAQVQLVQSGAEVKKPGASVKVSCKASGYTFSGYSTHWLRQVPGQGLEWIGWDNPSSGDTTYAENFRGRVTLTRDTSITTDYLEVRGLRSDDTAVYYCARGGDDYSFDHWGQGTLVTVSSGGGGSGGGGSGGGGSSYELTQPPSASGTPGQRVTISCSGSSSNIGSNTVNWYQQLPGTAPKLLIYSNNQRPSGVPDRFSGSKSGTSASLAISGLQSEDEADYYCAAWDARLTGPLFGGGTKLSVLRQPKAAPSAAAEQKLISEEDL

SEQ ID NO: 15 - protein, 264 aa, Homo sapiens
MAQVQLVQSGAEVKKPGASVKVSCKASGYTFSGYSTHWLRQVPGQGLEWIGWDNPSSGDTTYAENFRGRVTLTRDTSITTDYLEVRGLRSDDTAVYYCARGGDDYSFDHWGQGTLVTVSSGGGGSGGGGSGGGGSSSELTQDPAVSVALGQTVTITCQGGGLRNYYASWYQQKPGQAPVLLVYGRDNRPSGIPDRFSGSSSGNTASLTITGAQAEDEADYYCNSRDSSGNHLVFGGGTKLTVLGQPKATPSAAAEQKLISEEDL

SEQ ID NO: 16 - protein, 265 aa, Homo sapiens
MAQVQLVQSGAEVKKPGASVKVSCKASGYTFSGYSTHWLRQVPGQGLEWIGWDNPSSGDTTYAENFRGRVTLTRDTSITTDYLEVRGLRSDDTAVYYCARGGDDYSFDHWGQGTLVTVSSGGGGSGGGGSGGGGSQSALTQPASVSGSPGQSITISCTGTNSDVGIYNLVSWYQQHPGKAPKLMIYDVSNRPSGVSSRFSGSNSGNTATLTISGLQAEDEADYYCSAHAGDNTQFGGGTKLTVLSQPKAAPSAAAEQKLISEEDL

SEQ ID NO: 17 - protein, 307 aa, Homo sapiens
  MOD_RES (237): Variable amino acid
EQLDHDYAKLACKFYFKETVIMKYLLPTAAAGLLLLAAQPAMAQVQLVQSGAEVKKPGASVKVSCKASGYTFSGYSTHWLRQVPGQGLEWIGWDNPSSGDTTYAENFRGRVTLTRDTSITTDYLEVRGLRSDDTAVYYCARGGDDYSFDHWGQGTLVTVSSGGGGSGGGGSGGGGSQSALTQPASVSGSPGQSITISCTGTSSDLGGHNFVSWYQQHPGKAPKLMIYDVFNRPSGVXSRFXGSKSGNTASLTISGLQAEDEADYFCSSYTITSIVVFGGGTKLTVLGQPKATPSAAAEQKLISEEDL

SEQ ID NO: 18 - protein, 266 aa, Homo sapiens
MAQVQLVQSGAEVKKPGASVKVSCKASGYTFSGYSTHWLRQVPGQGLEWIGWDNPSSGDTTYAENFRGRVTLTRDTSITTDYLEVRGLRSDDTAVYYCARGGDDYSFDHWGQGTLVTVSSGGGGSGGGGSGGGGSQSVLTQPPSVSVAPGKTARIPCGGNNSGTKSVHWYQQKPGQAPVLVIYDDRVRPSGIPERFSGSNSGDTATLTISRVAAGDEADYYCQVSDGSGDPPTWVFGGGTRLTVLGQPKAAPSAAAEQKLISEEDL

SEQ ID NO: 19 - protein, 266 aa, Homo sapiens
MAQVQLVQSGAEVKKPGSSVKVSCKASGYTFSGYSTHWLRQVPGQGLEWIGWDNPSSGDTTYAENFRGRVTLTRDTSITTDYLEVRGLRSDDTAVYYCARGGDDYSFDHWGQGTLVTVSSSGGGSGGGGSGGGGSQTVVTQEPSFSVSPGGTITLTCGLSSGSVFTSYYPSWYQQTPGQAPRTLIYSTNTRSSGVPDRFSGSILGNKAALTITGAQADDESDYYCVLYMGSGIGVFGTGTKVTVLGQPKAAPSAAAEQKLISEEDL

SEQ ID NO: 20 - protein, 274 aa, Homo sapiens
MAQVQLVQSGAEVKEPGSSVKVSCKASGGTFSNYPISWVRQAPGQGLEWMGGIIPITNSPGYAQKFQGRVTISADESTGTVYMELSSLRSEDTAIYYCAKDPNRYESGVLHYWHGLDVWGQGTTVTVSSGGGGSGGGGSGGGGSLPVLTQPPSASGTPGQTVTLSCSGSSSNIGSNTVNWYQQLPGTAPKLLIYSNNQRPSGVPDRFSASKSGTSASLAITGLQAEDEADYFCAAWDDSLVYVFGTGTKVTVLGQPKAAPSAAAEQKLISEEDL

SEQ ID NO: 21 - protein, 268 aa, Homo sapiens
MAQVQLVQSGAEVKKPGSSVKVSCKASGGTFSSYAISWVRQAPGQGLEWMGWMNSNTGDTGYAQKFQGRVTMTRNTSTSTAYMELSSLRSEDTAVYYCAKISNYHYYAMDVWGQGTLVTVSSGGGGSGGGGSGGGGSLPVLTQPPSASGTSGQTVTISCSGGSSNIGSHLVTWYQQFPGTAPKVLIHTNDQRPSGVPDRISGSKSGTSASLAISGLQSDDEGDYYCAAWDDSLNGYVFGTGTKVTVLGQPKATPSAAAEQKLISEEDL

SEQ ID NO: 22 - protein, 268 aa, Homo sapiens
MAEVQLVQSGAEVKKPGASVKVSCKASGYTFTSYGISWVRQAPGQGLEWLGWINPNSGDTVYSQKFQGRVTMTSDKSVSTAYMELSSLRSDDTAVYYCASPGKNYYYGMDVWGQGTLVTVSSGGGGSGGGGSGGGGSQPVLTQPPSASGTPGQRVTISCSGSSSNIGSNLIYWYQQLPGTAPKLLIYSNNQRPSGVPDRFSGSKSGTSASLAISGLRSEDEADYFCSAWDDSLGGEVFGTGTKVNVLGQPKAAPSAAAEQKLISEEDL

SEQ ID NO: 23 - protein, 118 aa, Homo sapiens
QVQLVQSGAEVKKPGASVKVSCKASGYTFSGYSTHWLRQVPGQGLEWIGWDNPSSGDTTYAENFRGRVTLTRDTSITTDYLEVRGLRSDDTAVYYCARGGDDYSFDHWGQGTLVTVSS

SEQ ID NO: 24 - protein, 118 aa, Homo sapiens; identical to SEQ ID NO: 23

SEQ ID NO: 25 - protein, 118 aa, Homo sapiens; identical to SEQ ID NO: 23

SEQ ID NO: 26 - protein, 118 aa, Homo sapiens; identical to SEQ ID NO: 23

SEQ ID NO: 27 - protein, 118 aa, Homo sapiens; identical to SEQ ID NO: 23

SEQ ID NO: 28 - protein, 118 aa, Homo sapiens; identical to SEQ ID NO: 23

SEQ ID NO: 29 - protein, 118 aa, Homo sapiens; identical to SEQ ID NO: 23

SEQ ID NO: 30 - protein, 118 aa, Homo sapiens
QVQLVQSGAEVKKPGSSVKVSCKASGYTFSGYSTHWLRQVPGQGLEWIGWDNPSSGDTTYAENFRGRVTLTRDTSITTDYLEVRGLRSDDTAVYYCARGGDDYSFDHWGQGTLVTVSS

SEQ ID NO: 31 - protein, 127 aa, Homo sapiens
QVQLVQSGAEVKEPGSSVKVSCKASGGTFSNYPISWVRQAPGQGLEWMGGIIPITNSPGYAQKFQGRVTISADESTGTVYMELSSLRSEDTAIYYCAKDPNRYESGVLHYWHGLDVWGQGTTVTVSS

SEQ ID NO: 32 - protein, 120 aa, Homo sapiens
QVQLVQSGAEVKKPGSSVKVSCKASGGTFSSYAISWVRQAPGQGLEWMGWMNSNTGDTGYAQKFQGRVTMTRNTSTSTAYMELSSLRSEDTAVYYCAKISNYHYYAMDVWGQGTLVTVSS

SEQ ID NO: 33 - protein, 120 aa, Homo sapiens
EVQLVQSGAEVKKPGASVKVSCKASGYTFTSYGISWVRQAPGQGLEWLGWINPNSGDTVYSQKFQGRVTMTSDKSVSTAYMELSSLRSDDTAVYYCASPGKNYYYGMDVWGQGTLVTVSS

SEQ ID NO: 34 - protein, 105 aa, Homo sapiens
SSELTQDPAVSVALGQTVRITCRGDSLRSYYASWYQQKPGQAPVLVIYGENNRPSGIPDRFSGSSSGDTASLTITGAQAEDEADYYCNSRDSSDHLLLFGGGTKL

SEQ ID NO: 35 - protein, 111 aa, Homo sapiens
SYELTQPPSASGTTGQRVTISCSGSSSNIGSNTVNWYQQLPGTAPKLLIYSNNQRPSGVPDRLSGSKSGTSASLAISGLQSEDEADYYCAAWDARLTGPLFGGGTKLSVLR

SEQ ID NO: 36 - protein, 111 aa, Homo sapiens
SYELTQPPSASGTPGQRVTISCSGSSSNIGSNTVNWYQQLPGTAPKLLIYSNNQRPSGVPDRFSGSKSGTSASLAISGLQSEDEADYYCAAWDARLTGPLFGGGTKLSVLR

SEQ ID NO: 37 - protein, 109 aa, Homo sapiens
SSELTQDPAVSVALGQTVTITCQGGGLRNYYASWYQQKPGQAPVLLVYGRDNRPSGIPDRFSGSSSGNTASLTITGAQAEDEADYYCNSRDSSGNHLVFGGGTKLTVLG

SEQ ID NO: 38 - protein, 110 aa, Homo sapiens
QSALTQPASVSGSPGQSITISCTGTNSDVGIYNLVSWYQQHPGKAPKLMIYDVSNRPSGVSSRFSGSNSGNTATLTISGLQAEDEADYYCSAHAGDNTQFGGGTKLTVLS

SEQ ID NO: 39 - protein, 111 aa, Homo sapiens
  MOD_RES (61): Variable amino acid
QSALTQPASVSGSPGQSITISCTGTSSDLGGHNFVSWYQQHPGKAPKLMIYDVFNRPSGVXSRFXGSKSGNTASLTISGLQAEDEADYFCSSYTITSIVVFGGGTKLTVLG

SEQ ID NO: 40 - protein, 110 aa, Homo sapiens
QSVLTQPPSVSVAPGKTARIPCGGNNSGTKSVHWYQQKPGQAPVLVIYDDRVRPSGIPERFSGSNSGDTATLTISRVAAGDEADYYCQVSDGSGDPPTWVFGGGTRLTVL

SEQ ID NO: 41 - protein, 111 aa, Homo sapiens
QTVVTQEPSFSVSPGGTITLTCGLSSGSVFTSYYPSWYQQTPGQAPRTLIYSTNTRSSGVPDRFSGSILGNKAALTITGAQADDESDYYCVLYMGSGIGVFGTGTKVTVLG

SEQ ID NO: 42 - protein, 110 aa, Homo sapiens
LPVLTQPPSASGTPGQTVTLSCSGSSSNIGSNTVNWYQQLPGTAPKLLIYSNNQRPSGVPDRFSASKSGTSASLAITGLQAEDEADYFCAAWDDSLVYVFGTGTKVTVLG

SEQ ID NO: 43 - protein, 111 aa, Homo sapiens
LPVLTQPPSASGTSGQTVTISCSGGSSNIGSHLVTWYQQFPGTAPKVLIHTNDQRPSGVPDRISGSKSGTSASLAISGLQSDDEGDYYCAAWDDSLNGYVFGTGTKVTVLG

SEQ ID NO: 44 - protein, 111 aa, Homo sapiens
QPVLTQPPSASGTPGQRVTISCSGSSSNIGSNLIYWYQQLPGTAPKLLIYSNNQRPSGVPDRFSGSKSGTSASLAISGLRSEDEADYFCSAWDDSLGGEVFGTGTKVNVLG

SEQ ID NO: 45 - DNA, 354 bp, Homo sapiens
caggtgcagctggtgcagtctggggctgaggtgaagaagcctggggcctcagtgaaagtctcctgcaaggcttctggatacaccttcagcggctactctacacactggctgcgacaggtccctggacagggacttgagtggattggatgggacaaccctagtagtggtgacacgacctatgcagagaattttcggggcagggtcaccctgaccagggacacgtccatcaccacagattacttggaagtgaggggtctaagatctgacgacacggccgtctattattgtgccagaggcggagatgactacagctttgaccattggggtcagggcaccctggtcaccgtctcctca

SEQ ID NO: 46 - DNA, 354 bp, Homo sapiens
caggtgcagctggtgcagtctggggctgaggtgaagaagcctggggcctcagtgaaagtctcctgcaaggcttctggatacaccttcagcggctactctacacactggctgcgacaggtccctggacagggacttgagtggattggatgggacaaccctagtagtggtgacacgacctatgcagagaattttcggggcagggtcaccctgaccagggacacgtccatcaccacagattacttggaagtgaggggtctaagatctgacgacacggccgtctattattgtgccagaggcggagatgactacagctttgaccattggggtcagggaaccctggtcaccgtctcctca

SEQ ID NO: 47 - DNA, 354 bp, Homo sapiens; identical to SEQ ID NO: 46

SEQ ID NO: 48 - DNA, 354 bp, Homo sapiens; identical to SEQ ID NO: 46

SEQ ID NO: 49 - DNA, 354 bp, Homo sapiens; identical to SEQ ID NO: 46

SEQ ID NO: 50 - DNA, 354 bp, Homo sapiens; identical to SEQ ID NO: 46

SEQ ID NO: 51 - DNA, 354 bp, Homo sapiens; identical to SEQ ID NO: 46

SEQ ID NO: 52 - DNA, 354 bp, Homo sapiens
caggtgcagctggtgcagtctggggctgaggtgaagaagcctgggtcgtcggtgaaggtctcctgcaaggcttctggatacaccttcagcggctactctacacactggctgcgacaggtccctggacagggacttgagtggattggatgggacaaccctagtagtggtgacacgacctatgcagagaattttcggggcagggtcaccctgaccagggacacgtccatcaccacagattacttggaagtgaggggtctaagatctgacgacacggccgtctattattgtgccagaggcggagatgactacagctttgaccattggggtcagggcaccctggtcaccgtctcctca

SEQ ID NO: 53 - DNA, 381 bp, Homo sapiens
caggtgcagctggtgcagtctggggctgaggtgaaggagcctggatcttcagtgaaagtctcctgtaaggcttctggaggcaccttcagcaattatcctatcagttgggtgcgacaggcccctggacaagggcttgagtggatgggagggatcatccccatcactaattcgccaggctatgcacaaaagttccagggcagagttacaatttccgcggacgaatcgacgggcacagtctacatggagctgagcagcctgagatctgaggacacggccatatattactgtgcaaaagatccaaatcgctatgagagtggtgtactccactattggcacggtttggacgtctggggccaagggaccacggtcaccgtctcctca

SEQ ID NO: 54 - DNA, 360 bp, Homo sapiens
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggatggatgaattctaacactggtgacacaggctatgcacagaagttccagggcagagtcaccatgaccaggaacacctccacaagcacagcctatatggagctgagcagcctgagatccgaggacacggccgtctattactgtgcgaaaatctccaactaccactattacgctatggacgtctggggccaaggaaccctggtcaccgtctcctca

SEQ ID NO: 55 - DNA, 360 bp, Homo sapiens
gaggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggctgggctggatcaaccctaacagtggtgacacagtctattcacagaagtttcagggcagggtcaccatgaccagcgacaagtccgtcagcacagcctacatggaactgagcagcctgagatccgacgacacggccgtatattactgtgcctcccctgggaaaaattactactacggtatggacgtctggggccaaggcaccctggtcaccgtctcctca

SEQ ID NO: 56 - DNA, 327 bp, Homo sapiens
tcttctgagctgactcaggacccagctgtgtctgtggccttgggacagacagtcaggatcacatgccgaggagacagcctcagaagttattatgcaagctggtaccaacagaagccaggacaggcccctgtacttgtcatctatggtgaaaacaaccgaccctcagggatcccagaccgattctctggctccagctcaggagacacagcttccttgaccatcactggggctcaggcggaagatgaggctgactattactgtaactcccgggacagcagtgatcaccttctcctattcggtggagggaccaagttgaccgtcctaggt

SEQ ID NO: 57 - DNA, 333 bp, Homo sapiens
tcctatgagctgactcagccaccatcagcgtctgggaccaccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattatctggctccaagtctggcacctcagcctccctggccatcagtggactccagtctgaagatgaggccgattattactgtgctgcgtgggatgcccgcctgactggtcccctcttcggcggggggaccaagctaagcgtcctacgt

SEQ ID NO: 58 - DNA, 333 bp, Homo sapiens
tcctatgagctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtggactccagtctgaagatgaggccgattattactgtgctgcgtgggatgcccgcctgactggtcccctcttcggcggggggaccaagctaagcgtcctacgt

SEQ ID NO: 59 - DNA, 327 bp, Homo sapiens
tcttctgagctgactcaggaccctgctgtgtctgtggccttggggcagacagtcacgatcacatgtcaaggaggcggcctcagaaattattatgcaagttggtaccaacagaagccgggacaggcccctgtccttctcgtctatggaagagacaaccggccctcagggatcccagaccgattctctggctccagctcaggaaacacagcttccttgaccatcactggggctcaggcggaagatgaggctgactattactgtaactcccgggacagcagtggtaaccatctggtgttcggcggagggaccaagctgaccgtcctaggt

SEQ ID NO: 60 - DNA, 330 bp, Homo sapiens
cagtctgccctgactcagcctgcctccgtgtctgggtctcctggacagtcgatcaccatctcctgcactggaaccaacagtgatgttggaatttataaccttgtctcctggtaccaacagcacccaggcaaagcccccaaactcatgatttatgatgtcagtaatcggccctcaggggtttctagtcgcttctctggctccaactctgggaacacggccaccctgaccatctctgggctccaggctgaagatgaggctgattattattgcagcgcacatgcaggcgacaacacccaattcggcggagggaccaagctgaccgtcctaagt

SEQ ID NO: 61 - DNA, 333 bp, Homo sapiens
  modified_base (182): a, c, g, t, unknown, or other
cagtctgccctgactcagcctgcctccgtgtctgggtctcctggacagtcgatcaccatctcctgcactggaaccagcagtgaccttggtggtcacaactttgtctcctggtaccaacagcacccaggcaaagcccccaaactcatgatttatgatgtctttaatcggccctcaggggtttntagtcgnttctntggctccaagtctggcaacacggcctccctgaccatctctgggctccaggctgaggacgaggctgattatttctgcagctcatatacaatcaccagcatcgtggtcttcggcggagggaccaagctgaccgtcctaggt

SEQ ID NO: 62 - DNA, 333 bp, Homo sapiens
cagtctgtgctgactcagccaccctcagtgtcagtggccccaggaaagacggccaggattccctgtgggggaaacaacagtggaactaaaagtgtgcactggtaccagcagaagccaggccaggcccctgtgctggtcatctatgatgatagagtccggccctcagggatccctgagcgattctctggctccaactctggggacacggccaccctgaccatcagcagggtcgcagccggggatgaggccgactattactgtcaggtgtcggatggtagtggtgatcctcccacttgggtgttcggcggagggaccaggctgaccgtcctaggt

SEQ ID NO: 63 - DNA, 333 bp, Homo sapiens
cagactgtggtgactcaggagccatcgttctcagtgtcccctggagggaccatcacactcacttgtggcttgagctctggctcagtctttactagttactaccccagctggtaccagcagaccccaggccaggctccacgcacgctcatctacagcacaaacactcgctcttctggggtccctgatcgcttctctggctccatccttgggaacaaagctgccctcaccatcacgggggcccaggcagatgatgaatctgattattactgtgtcctgtatatgggtagtggcattggggtcttcggaactgggaccaaggtcaccgtcctaggt

SEQ ID NO: 64 - DNA, 330 bp, Homo sapiens
ctgcctgtgctgactcagccaccctcagcgtcggggacccccgggcagacggttaccctctcttgttctggaagcagctccaacatcggaagtaatactgtaaactggtaccagcagctcccaggaacggcccccaaactcctcatctatagtaataatcagcggccctcaggggtccctgaccgattctctgcctccaagtctggcacctcagcctccctggccatcactgggctccaggctgaggatgaggctgattatttctgtgcagcatgggatgacagcctggtttatgtcttcggaactgggaccaaggtcaccgtcctaggt

SEQ ID NO: 65 - DNA, 333 bp, Homo sapiens
ctgcctgtgctgactcagccaccctcagcgtctgggacctccgggcagacggtcaccatctcctgttctggagggagctccaacatcggaagtcatcttgtaacctggtaccagcagtttccagggacggcccccaaagtcctcatacatactaatgatcagcgaccctctggggtccctgaccgaatctctggctccaagtctggcacctcagcctccctggccatcagtggactccagtctgacgatgagggtgactattattgtgcagcatgggatgacagcctcaatggttatgtcttcggaactgggaccaaggtcaccgtcctgggt

SEQ ID NO: 66 - DNA, 333 bp, Homo sapiens
cagcctgtgctgactcagccaccctcagcgtctgggacccccgggcagagggtcaccatctcttgttctggaagcagctccaacatcggaagtaatcttatatattggtaccagcagctcccaggaacggcccccaaactcctcatctatagtaataatcagcggccctcaggggtccctgaccgattctctggctccaagtctggcacctcagcctccctggccatcagtgggctccggtccgaggatgaggctgattatttctgttcagcttgggatgacagcctgggtggcgaggtcttcggaactgggaccaaggtcaacgtcctaggt

SEQ ID NO: 67 - DNA, 801 bp, Homo sapiens
ctacagatcttcctctgagatgagtttttgttctgcggccgccgagggggcggccttgggctgacgtaggacgcttagcttggtccccccgccgaagaggggaccagtcaggcgggcatcccacgcagcacagtaataatcggcctcatcttcagactggagtccactgatggccagggaggctgaggtgccagacttggagccagataatcggtcagggacccctgagggccgctgattattactatagatgaggagtttgggggccgttcctgggagctgctggtaccagtttacagtattacttccgatgttggagctgcttccagaacaagagatggtgaccctctgcccggtggtcccagacgctgatggtggctgagtcagctcataggagctgccaccaccgccagaaccaccacctccggaaccgccgccacctgaggagacggtgaccagggttccctgaccccaatggtcaaagctgtagtcatctccgcctctggcacaataatagacggccgtgtcgtcagatcttagacccctcacttccaagtaatctgtggtgatggacgtgtccctggtcagggtgaccctgccccgaaaattctctgcataggtcgtgtcaccactactagggttgtcccatccaatccactcaagtccctgtccagggacctgtcgcagccagtgtgtagagtagccgctgaaggtgtatccagaagccttgcaggagactttcactgaggccccaggcttcttcacctcagccccagactgcaccagctgcacctgggccat

SEQ ID NO: 68 - protein, 118 aa, Homo sapiens; identical to SEQ ID NO: 23

SEQ ID NO: 69 - protein, 118 aa, Homo sapiens; identical to SEQ ID NO: 23

SEQ ID NO: 70 - protein, 118 aa, Homo sapiens; identical to SEQ ID NO: 23

SEQ ID NO: 71 - protein, 118 aa, Homo sapiens; identical to SEQ ID NO: 23

SEQ ID NO: 72 - protein, 118 aa, Homo sapiens; identical to SEQ ID NO: 23

SEQ ID NO: 73 - protein, 118 aa, Homo sapiens; identical to SEQ ID NO: 23

SEQ ID NO: 74 - protein, 118 aa, Homo sapiens; identical to SEQ ID NO: 23

SEQ ID NO: 75 - protein, 118 aa, Homo sapiens; identical to SEQ ID NO: 30

SEQ ID NO: 76 - protein, 127 aa, Homo sapiens
QVQLVQSGAEVKEPGSSVKVSCKASGGTFSNYPISWVRQAPGQGLEWMGGIIPITNSPGYAQKFQGRVTISADESTGTVYMELSSLRSEDTAIYYCARDPNRYESGVLHYWHGLDVWGQGTLVTVSS

SEQ ID NO: 77 - protein, 120 aa, Homo sapiens; identical to SEQ ID NO: 32

SEQ ID NO: 78 - protein, 120 aa, Homo sapiens; identical to SEQ ID NO: 33

SEQ ID NO: 79 - protein, 59 aa, Dengue virus type 1
QEGAMHTALTGATEIQTSGTTKIFAGHLKCRLKMDKLTLKGMSYVMCTGSFKLEKEVAE

SEQ ID NO: 80 - protein, 59 aa, Dengue virus type 3
QEGAMHTALTGATEIQTSGGTSIFAGHLKCRLKMDKLKLKGMSYAMCLNTFVLKKEVSE

SEQ ID NO: 81 - protein, 59 aa, Dengue virus type 2
QEGAMHTALTGATEIQMSSGNLLFTGHLKCRLRMDKLQLKGMSYSMCTGKFKVVKEIAE

SEQ ID NO: 82 - protein, 59 aa, Dengue virus type 4
QEGAMHSALTGATEVDSGDGNHMFAGHLKCKVRMEKLRIKGMSYTMCSGKFSIDKEMAE

SEQ ID NO: 83 - protein, 60 aa, West Nile virus
QEGALHQALAGAIPVEFSSNTVKLTSGHLKCRVKMEKLQLKGTTYGVCSKAFKFLGTPAD

SEQ ID NO: 84 - protein, 60 aa, St. Louis encephalitis virus
QEGALHTALAGAIPATVSGSTLTLQSGHLKCRAKLDKVKIKGTTYGMCDSAFTFSKNPTD

SEQ ID NO: 85 - protein, 20 aa, Artificial Sequence
  Description of Artificial Sequence Synthetic peptide
FNCLGMSNRDFLEGVSGATW

SEQ ID NO: 86 - protein, 20 aa, Artificial Sequence
  Description of Artificial Sequence Synthetic peptide
FLEGVSGATWVDLVLEGDSC

SEQ ID NO: 87 - protein, 20 aa, Artificial Sequence
  Description of Artificial Sequence Synthetic peptide
VDLVLEGDSCVTIMSKDKPT

SEQ ID NO: 88 - protein, 20 aa, Artificial Sequence
  Description of Artificial Sequence Synthetic peptide
VTIMSKDKPTIDVKMMNMEA

SEQ ID NO: 89 - protein, 20 aa, Artificial Sequence
  Description of Artificial Sequence Synthetic peptide
IDVKMMNMEAANLAEVRSYC

SEQ ID NO: 90 - protein, 20 aa, Artificial Sequence
  Description of Artificial Sequence Synthetic peptide
ANLAEVRSYCYLATVSDLST

SEQ ID NO: 91 - protein, 20 aa, Artificial Sequence
  Description of Artificial Sequence Synthetic peptide
YLATVSDLSTKAACPTMGEA

SEQ ID NO: 92 - protein, 20 aa, Artificial Sequence
  Description of Artificial Sequence Synthetic peptide
KAACPTMGEAHNDKRADPAF

SEQ ID NO: 93 - protein, 20 aa, Artificial Sequence
  Description of Artificial Sequence Synthetic peptide
HNDKRADPAFVCRQGVVDRG

SEQ ID NO: 94 - protein, 20 aa, Artificial Sequence
  Description of Artificial Sequence Synthetic peptide
VCRQGVVDRGWGNGCGLFGK

SEQ ID NO: 95 - protein, 20 aa, Artificial Sequence
  Description of Artificial Sequence Synthetic peptide
WGNGCGLFGKGSIDTCAKFA

SEQ ID NO: 96 - protein, 20 aa, Artificial Sequence
  Description of Artificial Sequence Synthetic peptide
GSIDTCAKFACSTKAIGRTI

SEQ ID NO: 97 - protein, 20 aa, Artificial Sequence
  Description of Artificial Sequence Synthetic peptide
CSTKAIGRTILKENIKYEVA

SEQ ID NO: 98 - protein, 20 aa, Artificial Sequence
  Description of Artificial Sequence Synthetic peptide
LKENIKYEVAIFVHGPTTVE

SEQ ID NO: 99 - protein, 20 aa, Artificial Sequence
  Description of Artificial Sequence Synthetic peptide
IFVHGPTTVESHGNYSTQVG

SEQ ID NO: 100 - protein, 20 aa, Artificial Sequence
  Description of Artificial Sequence Synthetic peptide
SHGNYSTQVGATQAGRFSIT

SEQ ID NO: 101 - protein, 20 aa, Artificial Sequence
  Description of Artificial Sequence Synthetic peptide
ATQAGRFSITPAAPSYTLKL

SEQ ID NO: 102 - protein, 20 aa, Artificial Sequence
  Description of Artificial Sequence Synthetic peptide
PAAPSYTLKLGEYGEVTVDC

SEQ ID NO: 103 - protein, 20 aa, Artificial Sequence
  Description of Artificial Sequence Synthetic peptide
GEYGEVTVDCEPRSGIDTNA

SEQ ID NO: 104 - protein, 20 aa, Artificial Sequence
  Description of Artificial Sequence Synthetic peptide
EPRSGIDTNAYYVMTVGTKT

SEQ ID NO: 105 - protein, 20 aa, Artificial Sequence
  Description of Artificial Sequence Synthetic peptide
YYVMTVGTKTFLVHREWFMD

SEQ ID NO: 106 - protein, 20 aa, Artificial Sequence
  Description of Artificial Sequence Synthetic peptide
FLVHREWFMDLNLPWSSAGS

SEQ ID NO: 107 - protein, 20 aa, Artificial Sequence
  Description of Artificial Sequence Synthetic peptide
LNLPWSSAGSTVWRNRETLM

SEQ ID NO: 108 - protein, 20 aa, Artificial Sequence
  Description of Artificial Sequence Synthetic peptide
TVWRNRETLMEFEEPHATKQ

SEQ ID NO: 109 - protein, 20 aa, Artificial Sequence
  Description of Artificial Sequence Synthetic peptide
EFEEPHATKQSVIALGSQEG

SEQ ID NO: 110 - protein, 20 aa, Artificial Sequence
  Description of Artificial Sequence Synthetic peptide
SVIALGSQEGALHQALAGAI

SEQ ID NO: 111 - protein, 20 aa, Artificial Sequence
  Description of Artificial Sequence Synthetic peptide
ALHQALAGAIPVEFSSNTVK

SEQ ID NO: 112 - protein, 20 aa, Artificial Sequence
  Description of Artificial Sequence Synthetic peptide
PVEFSSNTVKLTSGHLKCRV

SEQ ID NO: 113 - protein, 20 aa, Artificial Sequence
  Description of Artificial Sequence Synthetic peptide
LTSGHLKCRVKMEKLQLKGT

SEQ ID NO: 114 - protein, 20 aa, Artificial Sequence
  Description of Artificial Sequence Synthetic peptide
KMEKLQLKGTTYGVCSKAFK

SEQ ID NO: 115 - protein, 20 aa, Artificial Sequence
  Description of Artificial Sequence Synthetic peptide
TYGVCSKAFKFLGTPADTGH

SEQ ID NO: 116 - protein, 20 aa, Artificial Sequence
  Description of Artificial Sequence Synthetic peptide
FLGTPADTGHGTVVLELQYT

SEQ ID NO: 117 - protein, 20 aa, Artificial Sequence
  Description of Artificial Sequence Synthetic peptide
GTVVLELQYTGTDGPCKVPI

SEQ ID NO: 118 - protein, 20 aa, Artificial Sequence
  Description of Artificial Sequence Synthetic peptide
GTDGPCKVPISSVASLNDLT

SEQ ID NO: 119 - protein, 20 aa, Artificial Sequence
  Description of Artificial Sequence Synthetic peptide
SSVASLNDLTPVGRLVTVNP

SEQ ID NO: 120 - protein, 20 aa, Artificial Sequence
  Description of Artificial Sequence Synthetic peptide
PVGRLVTVNPFVSMATANAK

SEQ ID NO: 121 - protein, 20 aa, Artificial Sequence
  Description of Artificial Sequence Synthetic peptide
FVSMATANAKVLIELEPPFG

SEQ ID NO: 122 - protein, 20 aa, Artificial Sequence
  Description of Artificial Sequence Synthetic peptide
VLIELEPPFGDSYIVVGRGE

SEQ ID NO: 123 - protein, 20 aa, Artificial Sequence
  Description of Artificial Sequence Synthetic peptide
DSYIVVGRGEQQINHHWHKS

SEQ ID NO: 124 - protein, 20 aa, Artificial Sequence
  Description of Artificial Sequence Synthetic peptide
QQINHHWHKSGSSIGKAFTT

SEQ ID NO: 125 - protein, 109 aa, Homo sapiens
SSELTQDPAVSVALGQTVRITCRGDSLRSYYASWYQQKPGQAPVLVIYGENNRPSGIPDRFSGSSSGDTASLTITGAQAEDEADYYCNSRDSSDHLLLFGGGTKLTVLG

SEQ ID NO: 126 - protein, 111 aa, Homo sapiens; identical to SEQ ID NO: 35

SEQ ID NO: 127 - protein, 111 aa, Homo sapiens; identical to SEQ ID NO: 36

SEQ ID NO: 128 - protein, 109 aa, Homo sapiens; identical to SEQ ID NO: 37

SEQ ID NO: 129 - protein, 110 aa, Homo sapiens; identical to SEQ ID NO: 38

SEQ ID NO: 130 - protein, 111 aa, Homo sapiens; identical to SEQ ID NO: 39
  MOD_RES (61): Variable amino acid

SEQ ID NO: 131 - protein, 111 aa, Homo sapiens
QSVLTQPPSVSVAPGKTARIPCGGNNSGTKSVHWYQQKPGQAPVLVIYDDRVRPSGIPERFSGSNSGDTATLTISRVAAGDEADYYCQVSDGSGDPPTWVFGGGTRLTVLG

SEQ ID NO: 132 - protein, 111 aa, Homo sapiens; identical to SEQ ID NO: 41

SEQ ID NO: 133 - protein, 110 aa, Homo sapiens; identical to SEQ ID NO: 42

SEQ ID NO: 134 - protein, 112 aa, Homo sapiens
LPVLTQPPSASGTSGQTVTISCSGGSSNIGSHLVTWYQQFPGTAPKVLIHTNDQRPSGVPDRISGSKSGTSASLAISGLQSDDEGDYYCAAWDDSLNGYVLFGTGTKVTVLG

SEQ ID NO: 135 - protein, 111 aa, Homo sapiens; identical to SEQ ID NO: 44

SEQ ID NO: 136 - DNA, 19 bp, Artificial Sequence
  Description of Artificial Sequence Synthetic primer
agagggaaatcgtgctgac

SEQ ID NO: 137 - DNA, 20 bp, Artificial Sequence
  Description of Artificial Sequence Synthetic primer
caatagtgatgacctggccg

SEQ ID NO: 138 - DNA, 23 bp, Artificial Sequence
  Description of Artificial Sequence Synthetic probe
cactgccgcatcctcttcctccc

SEQ ID NO: 139 - protein, 15 aa, Artificial Sequence
  Description of Artificial Sequence Synthetic Gly-Ser linker
GGGGSGGGGSGGGGS

SEQ ID NO: 140 - protein, 6 aa, Artificial Sequence
  Description of Artificial Sequence Synthetic 6xHis tag
HHHHHH